SEQUENCE LISTING (46 sequences)

SEQ ID NO: 1 - DNA, 2655 bp, Lactobacillus johnsonii
  Lactobacillus johnsonii strain FI9785 gene number 111
atgtcagttgttgttaaatatgtagacctagacaataatttggttgaacttgcacattcaggagagttagagggaaaagtaggagaaagaatagactacaggacagaagatgagataagaaaaatactagctgctggttatgtactgattaataatccttttgatccaaatgataaagtaaatttctttaatgaagactctcaagaattcacgcttacttttaaacatggtaaagaagaaataactgccaaaaatttaaagtatggttgccatttagaggatgtccaaatgaaaggtgaacaagtagttcactatgttggactcgatcaaagtattcctgacaaggttactgaggttgcttttaatagaaaaattatttatgacaaagtcactaagaaaaaattattaactaatacctggcaaccggaaaagtattcctttcctttagttgcaagcccaactgtgatgaactatacacctaataaggcagtaattggtggagagacagcaactattcaacagccaaagtatgaatatgtagtcacttattcacccaataagaaaaatgctagaaggcaaaaagctgaaataaaatttattgacgttgatgataacaaccgcgaattagcaacttctggagaattaaaagggaaacctgaaaaagaaataccttataataccgcagaggttttgaaaaatttaacagctaagggatatgaagtagttactaatgattttgattctgagaaacagaatcctgtttttggaaatagtagagactatgttcaaatcttttttgtggttttaaagcataaaaaacaagttgttaattctgaacatccattttcaggaattgatgcaagtttgtacgaaaaagaagttcatcgaacaattcgtttttcaggaatagataataaaaagctagatgatgtagttcaaacagctattttaaaacggaacctaacagttgatctcgtaactaaaaaaattattccaggagaatacacaagtcaatggcatagtagtgaaacttacccgtcagtctccgtgcctgtagtttcgggatatcataccaagattagtgaagtagtagcctcaccagtagaaaaacaagatgtaactgaagaagttaaatactattcaaacggatatttaattcctgttgatgtaaatcataatgaattttcagaaatagataaaaagcaactaattaccaattcaattgatccaactaaggtcgttttaccggaattagacctaaaaaatattgtcttaaaacaaattaaagaagtggaaattgcagacccaagtcgtaattatgaaattccatatttattagttcataaatatgttgcagtcgatgaaaaacatccccaagaagtaataagtccggcatattaccgccgaattgttaccgcccgagttcattatcaaggagctggagaccagactcctccagatgccgaacaaacagtgcgttggacaagaacaattacttatgatgaagtaagtaaagaaattattgaaaacggtatgtatacgactgactgggtagctgataaagatatttttgaagcaactcctacacctgtaattaagggcttcgccgcaaatattggcttaattggcgaacacccagttacggaaactgacttaatggctacaattacctatactccgcttggaaagatgatcccagttgatgagcatggaaatgaaattaaaaatgctatgcacccaacatacgttaatgatccatatgatcctgtgcgtgtcctctttactgaagaagtaccagaagtgccaggctatgcaccagttaaaaatacaatttctgtcaatgatccttttacggatattaaggtgtcctataccttaaagccacgctatatccctgtgaatagcgaacatccttatcgtccgattaagccaactttgtatagtgtcccagttaaggaaataattaaatatcagggagcgggagagcagacaccaattactagaattcagggtgctaattggacgagaactttgactgttgatgagaatacgggtgaattagtagatgctggtaagtatacaacaggatggttggtagacaagaagcaatatatagccgtaaaaacaccagtcattgatggttatcatgcagacaaaaatataattgatgaagaaaaagtaaaaaaagccgatctaaacttcacggtaacttataaagccaatgggcggatagtaccagttgatgcaaaagggaatgttttaaatggtgtagaacagccaccatatgtaaccgatccaagcgatgcaactaaagtgattaaaacacaaggtgtaccgagaattatgaattatattccagatcgagcaacaattacggtaaaagatgctagcgaaaatacactggttaaatattatacttttgatgagatgagtgaattaaaagtagaatataaaaaaatagaacaagaaaaaagcaaagatataaaacctaagaagaaagcggaagaaaaaggtgcaaaggaggaagagcagtcattaaaaacagaggaagaaaagaaagcggagatggcttctaagaaagaagctacacaggtagatggacaaaatgaacaagagcgtaaagttttaaggcatatttttccatggatgaaatag

SEQ ID NO: 2 - DNA, 10206 bp, Lactobacillus johnsonii
  Lactobacillus johnsonii strain FI9785 gene number 1070
atgttattcaataagcattctgaaaccaagcaacgctttggaattcgtaaattaacaattggtgcatgttcagttttgttgtcaaccttattcttaactgtaaacaatggtcaacaggttaacgctgcaacaggtgatactgttactaatgcagataacaataagacagaaactgataatgaatctgttacaaaagaaaatgaagctaccaagacaaataatgatagcagtgttagcttaaataaagacgctaccgaggtaactaatacctcaaatggtactgatagtaccgaaaagcaaaaacctgaagtagaagataaaaatggtagtgctaaagtagatgatactaaggctactgaagctactgaagctactgaagataaatcatctgacaacagtagtgatcaaaagttaaccgaagataaaacagctaataataagctagctaaagcaactgttaataaattggcagttacaacagctaataataagctagctaaagcaactgttaataaattggcagttacaacagctaataatgagctagctaaagcagctgttgataaattggcagctacaacagatgattctacaccagtattatcacaatctactgaaaatggtgatatgactctctcgattagtttgccagaactttcaaataaagattatattcctcagacgattaaattaaatgcaactaatgtaaattctggtgacaaaattgttatcaaagtaaaaaaaagctcagcatatggttttgctaaagagaactttccaattggaacagttaaagaatctgatcagggtgattatcggatatttgagttagatattaatactactggtaaatttgaatataaaattacagctaaaaggaccaataattatcagggacaagccagtccgatgcaagatacaggagtaactacaaaagatattcagtggtccatcaacggtgaagatcaagcaccattgtcatttaaacagacaataattccagagttaatttcaaacggagttagtctagcaaaagcaactggagataaaaacactgattttacagaagttgctcctaatgaagattacaactttgtctattacatgggtgaaaatgatggtctggttcatgacggatcatatatggcaggcattgttaatagtgcggttaattatggtacgacaattacaattccagttcctgaaaattttcttttaaatcaggaagtaagtgataaggccaataaagagcgtggcttaactgactttaccatgacacaagatgggatgggcaaagatgtaattattactgttcctaaaggtcagagcgcgcaaggctggaatagcggtggtagatattatacattaactggtaaatttgtttatgatcagattccagaagaacctactaccgtgacagctaaaggagatagtgtcattgatcaaatttatacttcagatggtcagagaattaccgccaaaggaaagccatttagtgtaactatttctggtaaaaagggtagacctgcttcaggaccacttagtctttcagtttcaggagcgatttctaataatcaacttttattagattcaaatcctaataatgatccggtagctgtaaactggtttggttatagcaatgattacgataatattgagaatgccaaaataaagttagatttggcagatggcttatatgtaactgggattaagacccctaaagacctaggaactgtctataacaatattggtaatattcagagttatacatatgaaatgacattaactaatggtcagaaaattacgggtacagtaaaagctggtgatattgctaagtctactgccaccactacggatgcagataacaatcaaactattatcggtattagaagcattgtttttacaccagacactaatacaattggtaaggataccaaaacagataatctccctaaccctggacgtattgttgaccaagaggatagaatagatggtaaaaatccatcaaatgtctttattgccttgggacatttgagtcatacatatgataatggatcacaagtaaaggctaatgataaattaacttcaagtattactatttttggtagtaattttagaccagataggtataataacaacccaatagtttcatatacaagtagtaacattcaaacagtatttgatacatctcaatataaagcatcgctgagcacttatggtggacaaaattcaaccaatccaggaaaccaaaatgccggaggcatttctttaggcgacggtgatgggcgatcaaactatgattatatttttgaaccaatcttttattatgtaataccagataatgcggtatatagtggtggtgcaattagtagattaaattctaacggtaatgagtcgccagttcctgtagtgactacatatttcgttaatggtcaccaggtagttaagcttgattatagcaacacaaactattactataatactaaatatggaactaataatggtattcctttagataatgtaaataaccagacatcaaatactaggtcatgggaaatttatgcatacagtaaagatattcctcttttgaataatagttatacatctggtaagtttttaacacctgaaacagctgaaaagtcaggatcgactttaaaactagaccctagcaaattctactatattggtggaggaacctggacaattaatacagcatcagctgtagttctagccgatgctgcgaatggtaataaaaatcctaatggtctttatgttcaaaatggtacatctgatgataaaggatcagatgggatgaactttagagtaaatgtagtaaattacgatatgacaacagatctaaaagatctaactgcatttatcaatttaccagtcaaaggatacaacaataccacaactaacttctttttgagtggtccagttgatgttccagatggtacagttttgtattcaactagttcaacggatttaccatcaggtgtaggtacaaagacaccttcaactgataactttttaactaaagaacaagtcgaagctaagattgctagcggtgagatgagttgggctgatgttaagtcaattgcagtaaaatatgatactgtcaaggccaattccgcaactaaagacatttatatccacggaacagatccagatatcactaaagatgcaggtaaacgtgtacaattatcttggggcctatatggtggtaatgatatgccaccattggttaagaaaaatgcttctacgatagttatttccggatcatcaacaatcaacactagattgcattatgttgatccagaaggtaaggatcaatatgttgatgttccaacgatgtcaaaaacttataaagataattctgacaccatgcgggattcagattttaatgagaaaaatattcctgcaagcttaattcctaagggatatgaattagtgctacaaaatggtaaggccattaaatcaattattaataatggtggtacgacttgggcaactcatgcagaagacggttcagctcaatttggcaaagtagttctttataattttgatggtgatactgttcaatttgagttgacgccaaaaattgataaggctactcaaacagttacccgtactgtgcattttgttagtgatggtgataagccaccaaagcttcctaatgatgcatatgaaactgcaacaataactgaattgactaatgaagttactggtgaaaagcaatattcagctactattactgctggtggtgttacaactgatgctccagtagttgtaggtcaagatggtcagatatccatatctttcccagcaactacaattccagaagtgaataagtattatgttgttgatagtactaagagtgaagctgacgcaattagccctacatttactttcactaaagatggtgagttacctattgaaaatactgtgaaatatgctccagttaagcaagaattacaagtcaaagtttacgatgatgatgcagatgatcataatcaagcattagataccacagagacgggggctactgttgactttattggtaattctggaacagcctttccaactgatttgaagactaatttggaaaagttgaagcagtactacgagggtaagaattatattgttaaaacattgccagtagctaccggtaaatttgataatacacctaatggatcagggtctgatacgcaaattcaggtacttgaagttcatctaactcatgctaaagacgttaagactgaatatgctaaagctgttagaaatataacttatgaaggtgctggtgagcagactccagctactaagagtgatacttttgataatgctttttcaagaacgatcacaactgataaggtaactagaaaggatacaatttctgtttggactggctcacatacatttaatagtgtgaatagtccaagtgttgaaggctaccatccagataaggcatctgctggtaatattaaagcaaccgctgatagtttaaacgctgcagatgaaagtactctagctgaattattgaagacaggtgtagtagtttcagatcacgtaacatattcacctgataagcaagaagtcaaaattagagtttatgacgatacgactggtagcgagttaagtcctgttactgctcaaactgacataaggaaacaagggaaagatgttgaaattaatcttagcggtacttctaatgaaataattccaacagaatttggtaataacattgacttattgaaagagtactatcaatctaagggatataaatttatttcccataccttagtgccacaacactttgatcatacttctaatggaagtagtgaaactgatagtaatccacagtatattgatattcacttggaacatgatctgagtcttgaaaaagaaactaaaactgttactcggaccatcaattactatgatcaagataaaaaacaactgatcaatgatgccaataaaaaggttactgaaccacagactgttgttcaaaaagttaattttgcacgttatgctgttcgtgatgaggtaactaaccaaatcattggttatgcgactcctgatcaagtaactgttaaagatgatcacgcacaattagctaaaaagaatggatatactcctgtaactggtaaagctagtgatgctagagctagctttgtagtaactccagctgatagtaaatttgctggtcaaattaactacgatttatctaagtatggatataaagctccaactacaataaatggagatagttttgctcaagtagctgagttaactccacttctaactgatgctaattcaattgttaatgtttactatcgtgaaaaagtagtaacagttacggttgatgatccaccgacagttggcaaaaaggtacctggaacagatgctgaatttccaccaaatgaatggagcaaacttaatgcaaccagtacttcaactagaaccattcactacgtatacgatgataatacttttgcaaatggtgttgacgtaagtggaaaagctgtccctggattaaacgacatttttcaaaaagttaactttgctcaatctgctaaaatcaatctagtaaccggtgatgtttcatatcaaggagactggaaagcaattagttcaacaaccactaatcaacagggtgaagaagtggctaaccaagataatggtagctatgcaaaagttatttctccatcttctaagaacggttaccctgagcttaaaggttatacagctcaccaagaggtagttgatgctagtcaagcaactcatggtgttgatgttggacaggttttagttaaatacactgcaaatgattctttagtccaaatagaatatgtcgatcaagatactggtcttgcacttaaagttgatactaagaatggtaagagtggtgaaacattcgactattcaaccactgatacaattgcagactatgaaaagagaggatatgaattagttcatgatggatttactgaaaatgacggtaatcttaataataagacttttgattcatatgatgatgttccagatagtcaaagaccagaaacgattaatcagaagtggaaagtaactttaaaacataagaagattactgtaactaatgatgatccaaaagatccagatggtaagataactactgataagggatatgatcataactatccaactggtgtgagtgaaactgacttaaacaatacagttagaagaaatatttcctttatttatactgataaaccagaaggtagcaatcaagctttccctactgaaacacaagaagtagcttataaacgtcaagctacaattgatttagttaaacttgccaatggtgatagtgatgctgtttcatattcaaactggaaacctaaagaagcaggtaaagaaagttttgacagttatcaagttaaaccgattaagggctatgtagctaattatgaactggttccaagtgctgatgttcataaagaccaagatggtaaagctgaaaatggacaggatgttgtagttaagtattcaccagttgggaagattatccctattgacaaggatggtaacgaaattcctaatgcaccaactcctaaatacaacaacgatccaactgatccaactaagactacaacaaccgatgttcctgaaattccaggctatcatgctgaagtgccaaacgtaactccagataatccacttgaagatactaaggtagtttacgttaagaatacgcaggtcattgatttagtttatgttgatacaaagaccaatacaactttaactactcaaaacgacgtagcacatggtgattctggttcaacaatcccaactagtgtaaccgatactttgaattcaactattcaaagttacttagataaaggttatgtaattgatccatataaagttagcggaacagttcctgatacttttgactttagtgatcaaaccgctgatggtgcagataaagaacatcaagttgtgacaatctacttaactcatggtactgtaacagttactggtaatgatccaaagaccccaggtgaaccaattaatcctgaggatcctaatggaagtaagtatccatcagaagctggaaaagataatttagtaatttctagccaaaatattattcattattatgatgaggctggtaataagctacgtgatgataaagttactactgacgatagtacattaactagggaagtaactattgataaagttacaagtgacgtaatcagtactggtaaatggactggtggtaaggaatatgaaaatgttaatacaccggtagttaacggttactatactgatagagtcagtgccggtgcaagtaccgtaactcccgctgatgccgatcctaatagtggacgcgtacataatggagtaattactaatgaaactacagtgatttaccacccaatgggtcacattattccaatagataaatcaggtaagaaaattccaggtgcggaaacaccaatctttaataatgatcctaaggatccaactaaggcatctacgaccaattcaccaattattcctggatatcaccttgaaactcctagtgattcagcaatcactcctgatgaaccaggtaaggatcgcccagtagtttatgtagctgatactcaagaattgaaggttcaagtctttgatctagatggagatactcctgatgaaccgcttaagactgataaaacaggtgcaactgttgactttacaggtgactccttcactaacttctcaagtgatgtagcaactaacgtagattcattgattaagtactatactgatcgtggctacctagttaaaactaaaccaagcgatgaagaattatctggcaagtttgatggagatacaactcaaacacagtatctgaaattacaactagtgcatgataaagtgacagttagcggtgaagatgaaaatactccagcacctgatactccaattaatgagaatgatcctgatagtgttaagtatccgtctgacgtaagtaaagataatttagtaatttccagccaaaatattattcattattatgatgaggctggtaataagctacgtgatgataaagttactactgacgatagtacattaactagggaagtaactattgataaagttacaggtgacgtaattagtactggtaaatggactggtggtaaggaatatgaaaatgttaatacaccggtagttaacggttactatactgatagagccagtgccggtgcaagtaccgtaactcccgctgatgccgatcctaatagtggacgcgtacataatggagtaattactaatgaaactacagtgatttaccacccaatgggtcacattattccaatagataaatcaggtaagaaaattccaggtgcggaaacaccaatctttaataatgatcctaatgatccaaccaaaggatctacaactaattcaccaatcattcctggttatcatcttgaaactcctagtgattcagcaatcactcctgatgaaccaggtaaggatcgcccagtagtttatgtagctgatactcaagaattgaaggttcaagtctttgatctagatggagatactcctgatgaaccgcttaagactgataaaacaggtgcaactgttgactttacaggtgactccttcactaacttctcaagtgatgtagcaactaacgtagattcattgattaagtactatactgatcgtggctacctagttaaaactaaaccaagcgatgaagaattatctggcaagtttgatggagatacaactcaaacacagtatctgaaattacaactagtgcatgataaagtgacagttagcggtgaagatgaaaatactccagcacctgatactccaattaatgagaatgatcctgatagtgttaagtatccgtctgacgtaagtaaagataatttagtaatttctagtgaagtaataattcattatgaaggtgctggtgaaaaaacacctaaagatgtaattagaactgttgataagactttgactagaaccgtaactattgacaaagttactggtaaggtaaccaatactagtgattggtcaagcaatacaacagaatatgagtctgtattaactccacagattcagggatatacttctgataaggttcaagtagataaagtttccatcactaaggataatgctggtgaagctaagaatggaaagattacttacacatttaatgttgtttatacccctgataagcaacaattgattctaaaggtccatgatgaggatactcaaagctacttaggagatccggtaatttttgatggttatagtgatcaagaggttggcaattcttcagtagataagcttgaagaattaaagaagaaatatctcgatcttggctatgaaattgttgagatccctcaattggataagaactatgatgatacacaaaatgtaggtggagaaccagataaagaaccacaattttttgttcttaaggttaaacatcgaattgtaccagttactcctgatgatccaaagacacctgatgatatcattccagatagtaatcagaattatccaggaggattaactgaaactgatctttctagaactatcaatcgtacgatcgttgttaacttcccaagtggtacaagtcaagaaatcaaacaagtggttacctatactagaacggcaactgttgatgctgtaactaaggaagtaaaatattctgcttggacaactaagaatagcgattggcctgaacaagttgctccaactgttccaggatatgctccagataaggataaggttgacttgacttatgtcccagctgatggtaatgatgtgacagtggaaattaactatactgctgatcctgaaactgatgaaccaacaactccagttacaccaagtaagcctgaaaagccagctaagccggttaatcctactgagccaagtaagcctgaaaagccagctaagcagacaaagcctgaagttgtgaaacctgttaaagaaacaaaaggtaagcaattttatggtagcgctcaagctaaagttgacagtaaggctgctcggaatgttgtagacaaaaacaaagctaatggaactaacgataagaagttaccacaaactaatggtgaaagtaattggcagctttcacttctaggtgtgggtgtacttgcacttgcattattagttttgaagaaaaagagagacgatgaataa

SEQ ID NO: 3 - DNA, 633 bp, Lactobacillus johnsonii
  Lactobacillus johnsonii strain FI9785 gene number 1481
gtgattcttgatgcaattactggggaaatcaaatctgcgggtaaatggtctactgctaagtgggaaagatactcagctccagaatttaaaggttacactccagccccagctcaaattgatgaaactactgtgaacgaggatactcaggatacaactgttgagattagctatacagctaataagactccagagcaatcagatcacagcaaggacaaggatcaaggacaaaaaccacagactccagattcaaatgagaatggaaagcatcacttatcaactggtgaaaataccgagtctagtggttcaaccgctaagcctaaggtgctagctactgctaataaagtaactaagaatcttccacaaactggtgaaaataccgagtctagtggttcaaccgctaagcctaaggtgctagctactgctaataaagtaactaagaatcttccacaaactggtgaaaataccgagtctagtggttcaaccactaagcctaaggcgccagctactgctaataaagtaactaagaatcttccacaaactggtaaaaaagaaaaccaacttggactcttaggtattgtacttgcatctcttggagtctttggcttaggttggaaaaagagggaacataaatag

SEQ ID NO: 4 - DNA, 4071 bp, Lactobacillus johnsonii
  Lactobacillus johnsonii strain FI9785 gene number 1482
atgctatatagcaaccatgcggtattagccgatacgatgccaactaatacacatactacacctactggtcagactgcccaagcagatagcaaggtagcagaccatcaaggttctgaagggactgcagctcctgctgcatcaactacagctgatcaagaaaaaaaggtagcaactacagctcctgctgcatcaactacagctgatcaagaaaaaaaggtagcaactacagctgaagaaaaagataaagacaagcagacaaatgcgagtgaaactccgcaaaagcctaccaatgtcggcaaaaaagatactgaagtaacgtcgactaacgacgcaaaaccagctacacaaaagacaaccgccaagtctaaagtacgggtacgggctgcacgactacgatcagtaatgaaggtagcgaaggcagcacctattgctgaaacaccagatccaaccactccaagtgattcgactagtatggcaattgtcaacgataaaaacaagttggacaaaaactggatacagttggataatggaaaagctcaaatatactttactgttaaaggtaatgatcaaaaaccatacgttacttcttttatggataaggacgttaaatcgacactttacacaaagcgatttgatccaaatagtttagaacttaatattgaatatcaaaacggtgataaggatgcaggtcctggtttggtattagtctgggatgacaaatatcttcaattaaatactagcaggattggcgcagaagatttggtatatacaaccaattctggtgttaaggactatgttccagtaagtcctggcaatggcttttacaaatcttataatgaatggttaaaagagaagcaagatcctagcaagttgcatttgatctctctctggcagaaggttccagccaattcaacttttactgcaacattgccatttaagtatgttggtaatgctgatgtggtcaaatccactagagttgacgcttatttgaaaccatactccacaactttaacatttgatccattcgaattgcacccagagcaaattgatccagctccaaaaccaactactgatgatgccactacgaccacaggagtttacaacaacccattggttacatcttcagactggattaagatggttcatggtaatgctattggttactacactgctaaaggaaaagatggtaaaacatatgtaacaggatatgcggctcccaagggtaattcacaatcgatctttaatgtcgaccaaattgatttaaatagcttagaattccaccttctttatcacaatggtgacaaagaagttaaatccggactttggtttaactttgctgataataaagcgatagcaattgatactagtcgtttaggtgccgatggtccgcagttaaagagccaacaaggttggtcctataaatgggctattcatacaggtagtgacggaggtacttattataatagttgggcagaatatctcaaggctcacaacaatgtaggggaccaagcttctgagatttctatgacaggggttgctatcaagccagatgatacgattgaatttaatatcccagttaagtataccggtcagctgtctgccagaaccccatccttaactttctctccacatattaacgactacggtaagggtttcactcaaaactgggcagatctttactttacaactgtgaagaaggacttaagcgaggttaagcgggtagtcttgcttccgactgagttcatgggggaaaaacctggtgaatcagactggaagctcgttaaggaaatttccaaagatatgccaaatgggtgggatgcctttaaaattagcaacttcccaacctataagggatcattcaatatggattcactaccaataggggacaaaccatcgcttgtttacggcagtgcgcaatactttattgacctaagcaaaattcaagaaatttttgctaaccatggttacacggtcaagtatgctaacaacaacggtaaagctcaggaaatgccattctatgcatactccaccataccactggcaaaacgtgttgatgatgataaacctttcaataatgacccatctatctgggtaatccaagttcaagccgtgcctgctattattttaaataaggatcaacactatgtggcagatccaaatgctaagccatgggacaccacttcaatgattgatgagatttatgcagctgatgacgcaggacagcggaagacgcgcgatcaactaggaaagttgccaaagacggacgtggatattagttatgaataccaagctccaggggctgctacggccgctgcggttgataaggtcgacttgactaaggcgggggtatacactgtcacttacagtcatacttatgctgatgggcgagtagttaaggatagtcgcaaggtttatgtcgatggtgttaagccagaaaacaagaaaattactcgaacaattattgtccacgagccaaacggaactgaccaaacagttagccaaacagcaacagtgacacggaaagtgattcttgatgcaactactgggaaaatcaaatctgcgggtaaatggtctactgctaagtgggaagcatactcagctccagaatttaaaggttacactccagccccagcacaaattgatgaaactactgtgaacgagggcactcaagatacaactgttgagattagctatacagctaataagaccccagagcaaccagatcacaacgtcgatggtggtaagcaagaacacaagtacattactcgaacaattattgtccacacgccaaacggaactgaccaaaaagttatccaaaaagcaacattgacacgggaagtgattcttgatgcaactactggggaaatcaaatctgcgggcaaatggtctactgctaagtgggaaacatacttagctccagaatttaaaggttacactccagccccagcacaaattgatgaaactactgtgaacgagggcactcaagatacaactgttgaaattagctatacagctaataagaccccagagcaaccagatcacaacgtcgatggtggtaagcaagaaaccaagtacattactcgaacaattattgtccacacgccaaacggaactgaccaaacagttatccaaaaagcaacattgacacggaaagtgattcttgatgcaactactggggaaatcaaatctgcgggtaaatggtctactgctaagtgggaaacatactcagctccagaatttaaaggttacactccagccccagctcaaattgatgaaactactgtgaacgaggacactcaggatacaactgttgaaattagctatacagctaataagactccagagcaaccagatcacaacgtcgatggtggtaagcaagaaaccaagtacattactcgaacaattattgtccacgagccaaacggaactgaccaaacagttatccaaacagcaacagtgacacggaaagtgattcttgatgcaactactggggaaatcaaatctgcgggtaaatggtctactgctaagtgggaagcatactcagctccagaatttaaaggttacactccagccccagctcaaattgatgaaactactgtgaacgaggacactcaggatacaactgttgaaattagctatacagctaataagactccagagcaaccagatcacaacggtaagcaagaaaccaaggacattactcgaacaattattgtccacgagccaaacggaactgaccaaacagttatccaaaaagcaacattgacacggaaagtgattcttgatgcaactactggggaaatcaaatctgcgggtaagtggtctactgctaagtgggaaagatactcagctccagaatttaaaggttacactccagccccagctcaaattgaagaaactactgtgaacgaggatactcaggatacaactgttgagattagctatacagctaataagactccagagcaaccagatcacaacggtaagcaagaaaccaaggacattactcgaacaattatgtccacacgccaaacggagctgaccaaaaagttatccaaaaagcaacagtga

SEQ ID NO: 5 - DNA, 1776 bp, Lactobacillus johnsonii
  Lactobacillus johnsonii strain FI9785 gene number 1651
atgtacaatgtgaaatttttagtagttagtagtggtaaatctgatgttactgttaacgaaaacaaaactcctgttggaaatgcaagtacagttgtgggaagtacacgaattgaatattgtttaggaaatagtactgatatagatgccagttcaattggtgaaattattcctatttatacagaacagtctgtgattaagtactactaccggaacaaagatggaaaattggttgaaatcccaggcactgataaatactctaatgttaacgtatcaggctggactggccaagaatttgttgttgataacgttgatcaatataaacagctaattgatggcttttacttagatgattcaaatattcctacgggctcgtttactggaactctttctcaattcggagatggtagttactataagaaagtttactatagtgctggatataaagatgaaggcactattgatcaattaaaggtagattttacggtagtttaccgtcaaattgatgctaccggtaagatggaagttctgatgtatgatggcagcaatatgactaaggttattgagagtcatactgtagaagctggtaaatcagttaagtttttttataacaactacacagctagaaacccatttgtaactgactctgctcatgaagtacagtttgtgtataaagaattaggttccttaattttagttgatgaaaacgggaaagaacttcgtgcacctataaaatttaacaatgatccaactaaggctggactaacctctgcaccgattattgagggatatattgcttatattgaatacgtagttccagaagatacaggtaaagatatcacagttatctacactgcaaatgcagctaaggctgaaattatctatgttgatgagactacaggcaagcaattggaaactgcagtagttgacggtaagtataacgaaacaattaactacagcacagccgataagattaaatactatgaaagcttaggctatgaattagtcaaagatggttatgttggcggagaattaggtgaagacacaaagactttctatgtaacgttcaaacatggagcagttatagttactcctgatgataaatttaccgaagaggatccgattaatccagacaacccagatggaccaaaatatccatttgattcaattgctttagataaaacaattactcgtacagttaaatatgtttacgcagatgagactaaagcaaaagatgacgtagttcaaaagttaagattccgtggtacagccattattgataaagtaactggcgaagtcattatattagacgaaaatggcagaaagatttctgatggaattaaatggacagcacttgatggaactacatttatacaagtcatttctccagatattgctggctacactccagataggaaagaaattggtagccttgaaaatgtagatagtaatattgacgatatcattgaaactgtagtttacaataaggatatcgtagatccagaaggaccaaaggttccggctcaagttgaaactccaatagaatcaacgaatgatgctcctattcttccagaacaactttctcctgatactactattaataaagtagaaactgtagaaaacaaagaatctggtaaagtagaagcagctacaactgaggatgttgcattacctcaaacaggtcataaacattctaatgcaggacttattggactaggtttagcaaccattgcgtcaattttaggcttagcaggtactagaaaacgcaagaaggattaa

SEQ ID NO: 6 - protein, 884 aa, Lactobacillus johnsonii
  Lactobacillus johnsonii strain FI9785 gene number 111 gene product
MSVVVKYVDLDNNLVELAHSGELEGKVGERIDYRTEDEIRKILAAGYVLINNPFDPNDKVNFFNEDSQEFTLTFKHGKEEITAKNLKYGCHLEDVQMKGEQVVHYVGLDQSIPDKVTEVAFNRKIIYDKVTKKKLLTNTWQPEKYSFPLVASPTVMNYTPNKAVIGGETATIQQPKYEYVVTYSPNKKNARRQKAEIKFIDVDDNNRELATSGELKGKPEKEIPYNTAEVLKNLTAKGYEVVTNDFDSEKQNPVFGNSRDYVQIFFVVLKHKKQVVNSEHPFSGIDASLYEKEVHRTIRFSGIDNKKLDDVVQTAILKRNLTVDLVTKKIIPGEYTSQWHSSETYPSVSVPVVSGYHTKISEVVASPVEKQDVTEEVKYYSNGYLIPVDVNHNEFSEIDKKQLITNSIDPTKVVLPELDLKNIVLKQIKEVEIADPSRNYEIPYLLVHKYVAVDEKHPQEVISPAYYRRIVTARVHYQGAGDQTPPDAEQTVRWTRTITYDEVSKEIIENGMYTTDWVADKDIFEATPTPVIKGFAANIGLIGEHPVTETDLMATITYTPLGKMIPVDEHGNEIKNAMHPTYVNDPYDPVRVLFTEEVPEVPGYAPVKNTISVNDPFTDIKVSYTLKPRYIPVNSEHPYRPIKPTLYSVPVKEIIKYQGAGEQTPITRIQGANWTRTLTVDENTGELVDAGKYTTGWLVDKKQYIAVKTPVIDGYHADKNIIDEEKVKKADLNFTVTYKANGRIVPVDAKGNVLNGVEQPPYVTDPSDATKVIKTQGVPRIMNYIPDRATITVKDASENTLVKYYTFDEMSELKVEYKKIEQEKSKDIKPKKKAEEKGAKEEEQSLKTEEEKKAEMASKKEATQVDGQNEQERKVLRHIFPWMK

SEQ ID NO: 7 - protein, 3401 aa, Lactobacillus johnsonii
  Lactobacillus johnsonii strain FI9785 gene number 1070 gene product
MLFNKHSETKQRFGIRKLTIGACSVLLSTLFLTVNNGQQVNAATGDTVTNADNNKTETDNESVTKENEATKTNNDSSVSLNKDATEVTNTSNGTDSTEKQKPEVEDKNGSAKVDDTKATEATEATEDKSSDNSSDQKLTEDKTANNKLAKATVNKLAVTTANNKLAKATVNKLAVTTANNELAKAAVDKLAATTDDSTPVLSQSTENGDMTLSISLPELSNKDYIPQTIKLNATNVNSGDKIVIKVKKSSAYGFAKENFPIGTVKESDQGDYRIFELDINTTGKFEYKITAKRTNNYQGQASPMQDTGVTTKDIQWSINGEDQAPLSFKQTIIPELISNGVSLAKATGDKNTDFTEVAPNEDYNFVYYMGENDGLVHDGSYMAGIVNSAVNYGTTITIPVPENFLLNQEVSDKANKERGLTDFTMTQDGMGKDVIITVPKGQSAQGWNSGGRYYTLTGKFVYDQIPEEPTTVTAKGDSVIDQIYTSDGQRITAKGKPFSVTISGKKGRPASGPLSLSVSGAISNNQLLLDSNPNNDPVAVNWFGYSNDYDNIENAKIKLDLADGLYVTGIKTPKDLGTVYNNIGNIQSYTYEMTLTNGQKITGTVKAGDIAKSTATTTDADNNQTIIGIRSIVFTPDTNTIGKDTKTDNLPNPGRIVDQEDRIDGKNPSNVFIALGHLSHTYDNGSQVKANDKLTSSITIFGSNFRPDRYNNNPIVSYTSSNIQTVFDTSQYKASLSTYGGQNSTNPGNQNAGGISLGDGDGRSNYDYIFEPIFYYVIPDNAVYSGGAISRLNSNGNESPVPVVTTYFVNGHQVVKLDYSNTNYYYNTKYGTNNGIPLDNVNNQTSNTRSWEIYAYSKDIPLLNNSYTSGKFLTPETAEKSGSTLKLDPSKFYYIGGGTWTINTASAVVLADAANGNKNPNGLYVQNGTSDDKGSDGMNFRVNVVNYDMTTDLKDLTAFINLPVKGYNNTTTNFFLSGPVDVPDGTVLYSTSSTDLPSGVGTKTPSTDNFLTKEQVEAKIASGEMSWADVKSIAVKYDTVKANSATKDIYIHGTDPDITKDAGKRVQLSWGLYGGNDMPPLVKKNASTIVISGSSTINTRLHYVDPEGKDQYVDVPTMSKTYKDNSDTMRDSDFNEKNIPASLIPKGYELVLQNGKAIKSIINNGGTTWATHAEDGSAQFGKVVLYNFDGDTVQFELTPKIDKATQTVTRTVHFVSDGDKPPKLPNDAYETATITELTNEVTGEKQYSATITAGGVTTDAPVVVGQDGQISISFPATTIPEVNKYYVVDSTKSEADAISPTFTFTKDGELPIENTVKYAPVKQELQVKVYDDDADDHNQALDTTETGATVDFIGNSGTAFPTDLKTNLEKLKQYYEGKNYIVKTLPVATGKFDNTPNGSGSDTQIQVLEVHLTHAKDVKTEYAKAVRNITYEGAGEQTPATKSDTFDNAFSRTITTDKVTRKDTISVWTGSHTFNSVNSPSVEGYHPDKASAGNIKATADSLNAADESTLAELLKTGVVVSDHVTYSPDKQEVKIRVYDDTTGSELSPVTAQTDIRKQGKDVEINLSGTSNEIIPTEFGNNIDLLKEYYQSKGYKFISHTLVPQHFDHTSNGSSETDSNPQYIDIHLEHDLSLEKETKTVTRTINYYDQDKKQLINDANKKVTEPQTVVQKVNFARYAVRDEVTNQIIGYATPDQVTVKDDHAQLAKKNGYTPVTGKASDARASFVVTPADSKFAGQINYDLSKYGYKAPTTINGDSFAQVAELTPLLTDANSIVNVYYREKVVTVTVDDPPTVGKKVPGTDAEFPPNEWSKLNATSTSTRTIHYVYDDNTFANGVDVSGKAVPGLNDIFQKVNFAQSAKINLVTGDVSYQGDWKAISSTTTNQQGEEVANQDNGSYAKVISPSSKNGYPELKGYTAHQEVVDASQATHGVDVGQVLVKYTANDSLVQIEYVDQDTGLALKVDTKNGKSGETFDYSTTDTIADYEKRGYELVHDGFTENDGNLNNKTFDSYDDVPDSQRPETINQKWKVTLKHKKITVTNDDPKDPDGKITTDKGYDHNYPTGVSETDLNNTVRRNISFIYTDKPEGSNQAFPTETQEVAYKRQATIDLVKLANGDSDAVSYSNWKPKEAGKESFDSYQVKPIKGYVANYELVPSADVHKDQDGKAENGQDVVVKYSPVGKIIPIDKDGNEIPNAPTPKYNNDPTDPTKTTTTDVPEIPGYHAEVPNVTPDNPLEDTKVVYVKNTQVIDLVYVDTKTNTTLTTQNDVAHGDSGSTIPTSVTDTLNSTIQSYLDKGYVIDPYKVSGTVPDTFDFSDQTADGADKEHQVVTIYLTHGTVTVTGNDPKTPGEPINPEDPNGSKYPSEAGKDNLVISSQNIIHYYDEAGNKLRDDKVTTDDSTLTREVTIDKVTSDVISTGKWTGGKEYENVNTPVVNGYYTDRVSAGASTVTPADADPNSGRVHNGVITNETTVIYHPMGHIIPIDKSGKKIPGAETPIFNNDPKDPTKASTTNSPIIPGYHLETPSDSAITPDEPGKDRPVVYVADTQELKVQVFDLDGDTPDEPLKTDKTGATVDFTGDSFTNFSSDVATNVDSLIKYYTDRGYLVKTKPSDEELSGKFDGDTTQTQYLKLQLVHDKVTVSGEDENTPAPDTPINENDPDSVKYPSDVSKDNLVISSQNIIHYYDEAGNKLRDDKVTTDDSTLTREVTIDKVTGDVISTGKWTGGKEYENVNTPVVNGYYTDRASAGASTVTPADADPNSGRVHNGVITNETTVIYHPMGHIIPIDKSGKKIPGAETPIFNNDPNDPTKGSTTNSPIIPGYHLETPSDSAITPDEPGKDRPVVYVADTQELKVQVFDLDGDTPDEPLKTDKTGATVDFTGDSFTNFSSDVATNVDSLIKYYTDRGYLVKTKPSDEELSGKFDGDTTQTQYLKLQLVHDKVTVSGEDENTPAPDTPINENDPDSVKYPSDVSKDNLVISSEVIIHYEGAGEKTPKDVIRTVDKTLTRTVTIDKVTGKVTNTSDWSSNTTEYESVLTPQIQGYTSDKVQVDKVSITKDNAGEAKNGKITYTFNVVYTPDKQQLILKVHDEDTQSYLGDPVIFDGYSDQEVGNSSVDKLEELKKKYLDLGYEIVEIPQLDKNYDDTQNVGGEPDKEPQFFVLKVKHRIVPVTPDDPKTPDDIIPDSNQNYPGGLTETDLSRTINRTIVVNFPSGTSQEIKQVVTYTRTATVDAVTKEVKYSAWTTKNSDWPEQVAPTVPGYAPDKDKVDLTYVPADGNDVTVEINYTADPETDEPTTPVTPSKPEKPAKPVNPTEPSKPEKPAKQTKPEVVKPVKETKGKQFYGSAQAKVDSKAARNVVDKNKANGTNDKKLPQTNGESNWQLSLLGVGVLALALLVLKKKRDDE

SEQ ID NO: 8 - protein, 210 aa, Lactobacillus johnsonii
  Lactobacillus johnsonii strain FI9785 gene number 1481 gene product
VILDAITGEIKSAGKWSTAKWERYSAPEFKGYTPAPAQIDETTVNEDTQDTTVEISYTANKTPEQSDHSKDKDQGQKPQTPDSNENGKHHLSTGENTESSGSTAKPKVLATANKVTKNLPQTGENTESSGSTAKPKVLATANKVTKNLPQTGENTESSGSTTKPKAPATANKVTKNLPQTGKKENQLGLLGIVLASLGVFGLGWKKREHK

SEQ ID NO: 9 - protein, 1356 aa, Lactobacillus johnsonii
  Lactobacillus johnsonii strain FI9785 gene number 1482 gene product
MLYSNHAVLADTMPTNTHTTPTGQTAQADSKVADHQGSEGTAAPAASTTADQEKKVATTAPAASTTADQEKKVATTAEEKDKDKQTNASETPQKPTNVGKKDTEVTSTNDAKPATQKTTAKSKVRVRAARLRSVMKVAKAAPIAETPDPTTPSDSTSMAIVNDKNKLDKNWIQLDNGKAQIYFTVKGNDQKPYVTSFMDKDVKSTLYTKRFDPNSLELNIEYQNGDKDAGPGLVLVWDDKYLQLNTSRIGAEDLVYTTNSGVKDYVPVSPGNGFYKSYNEWLKEKQDPSKLHLISLWQKVPANSTFTATLPFKYVGNADVVKSTRVDAYLKPYSTTLTFDPFELHPEQIDPAPKPTTDDATTTTGVYNNPLVTSSDWIKMVHGNAIGYYTAKGKDGKTYVTGYAAPKGNSQSIFNVDQIDLNSLEFHLLYHNGDKEVKSGLWFNFADNKAIAIDTSRLGADGPQLKSQQGWSYKWAIHTGSDGGTYYNSWAEYLKAHNNVGDQASEISMTGVAIKPDDTIEFNIPVKYTGQLSARTPSLTFSPHINDYGKGFTQNWADLYFTTVKKDLSEVKRVVLLPTEFMGEKPGESDWKLVKEISKDMPNGWDAFKISNFPTYKGSFNMDSLPIGDKPSLVYGSAQYFIDLSKIQEIFANHGYTVKYANNNGKAQEMPFYAYSTIPLAKRVDDDKPFNNDPSIWVIQVQAVPAIILNKDQHYVADPNAKPWDTTSMIDEIYAADDAGQRKTRDQLGKLPKTDVDISYEYQAPGAATAAAVDKVDLTKAGVYTVTYSHTYADGRVVKDSRKVYVDGVKPENKKITRTIIVHEPNGTDQTVSQTATVTRKVILDATTGKIKSAGKWSTAKWEAYSAPEFKGYTPAPAQIDETTVNEGTQDTTVEISYTANKTPEQPDHNVDGGKQEHKYITRTIIVHTPNGTDQKVIQKATLTREVILDATTGEIKSAGKWSTAKWETYLAPEFKGYTPAPAQIDETTVNEGTQDTTVEISYTANKTPEQPDHNVDGGKQETKYITRTIIVHTPNGTDQTVIQKATLTRKVILDATTGEIKSAGKWSTAKWETYSAPEFKGYTPAPAQIDETTVNEDTQDTTVEISYTANKTPEQPDHNVDGGKQETKYITRTIIVHEPNGTDQTVIQTATVTRKVILDATTGEIKSAGKWSTAKWEAYSAPEFKGYTPAPAQIDETTVNEDTQDTTVEISYTANKTPEQPDHNGKQETKDITRTIIVHEPNGTDQTVIQKATLTRKVILDATTGEIKSAGKWSTAKWERYSAPEFKGYTPAPAQIEETTVNEDTQDTTVEISYTANKTPEQPDHNGKQETKDITRTIMSTRQTELTKKLSKKQQ

SEQ ID NO: 10 - protein, 591 aa, Lactobacillus johnsonii
  Lactobacillus johnsonii strain FI9785 gene number 1651 gene product
MYNVKFLVVSSGKSDVTVNENKTPVGNASTVVGSTRIEYCLGNSTDIDASSIGEIIPIYTEQSVIKYYYRNKDGKLVEIPGTDKYSNVNVSGWTGQEFVVDNVDQYKQLIDGFYLDDSNIPTGSFTGTLSQFGDGSYYKKVYYSAGYKDEGTIDQLKVDFTVVYRQIDATGKMEVLMYDGSNMTKVIESHTVEAGKSVKFFYNNYTARNPFVTDSAHEVQFVYKELGSLILVDENGKELRAPIKFNNDPTKAGLTSAPIIEGYIAYIEYVVPEDTGKDITVIYTANAAKAEIIYVDETTGKQLETAVVDGKYNETINYSTADKIKYYESLGYELVKDGYVGGELGEDTKTFYVTFKHGAVIVTPDDKFTEEDPINPDNPDGPKYPFDSIALDKTITRTVKYVYADETKAKDDVVQKLRFRGTAIIDKVTGEVIILDENGRKISDGIKWTALDGTTFIQVISPDIAGYTPDRKEIGSLENVDSNIDDIIETVVYNKDIVDPEGPKVPAQVETPIESTNDAPILPEQLSPDTTINKVETVENKESGKVEAATTEDVALPQTGHKHSNAGLIGLGLATIASILGLAGTRKRKKD

SEQ ID NO: 11 - DNA, 1005 bp, Lactobacillus johnsonii
  Lactobacillus johnsonii strain FI9785 gene number 1183 epsA,undefined product, positions 11136421114646 reverse
atggatcataagaatagtgataatgaattaagacatcgctcacatcatcaccgtcatcatcgacgtaaaaagttttggcgtatcttttggattgtattaggtgtcttcctagcagtagacatcattgcggttattatcgcttggcacaatatccatgttgccactaataatatgtataatccgatgagtaatgaaattagtgatcggaaggttagcgacacactgaaagataagaagccaatgagcttgcttttattgggaacagatacaggtgaatttggacgttcttacaaggggagaacagataccattatgatgatggttatcaatcctaagaccaataagacaacagttgtcagcttgcctcgagatatgaaagttaacttaccgggttatccagactattcaccagctaagattaatgcagcctatacttatggtggtgtagatgaaactgttaaaacgatcaagaagtacttcaatgttccaactgatgcttacgtcatggtaaacatgggaggacttgagaaggcaattgatcaagttggaggagtgacagttaagtcgccattaacatttgattatgaaggctatcatttcactaaggacgtaacttatcacatgaatgggaagaaggctttggcattcagtagaatgcggtatgacgacccacgaggagattatgggcgtcaagagagacaaagacttgtaattatggcattattaaagagttccatttcttataagacagtcgttaaccaagcattcttgaattcaatctctaagcaaactatgactaacttgacgtttgataatatggttgctttggctcagaactatcgccatgcaactgataatgtcacaactgaccatgctcaagggcaaggcgattgggagaatggcgttgcttatgagtcagttagtcgagcagaatgtcaaagaattagtaataagttacgtgctgccttagggcttaagccggaaaccttaaagacaggagaatag

SEQ ID NO: 12 - DNA, 864 bp, Lactobacillus johnsonii
  Lactobacillus johnsonii strain FI9785 gene number 1182 epsB,polymerization and chain length determination protein, positions 11127721113635 reverse
gtggaaaacagtacaaaaactgaaaatacaatcgatttacgtagattatggatgcttcttcgagcacatatctggtctattattctatgggcaattggattaggagcagtaggatttgttctagctgcttttgtggttgaacctaaatatacttcaactactcaaatcttagttaatcagaagcgaaatgcagttgacgcgggtcaagcatataatgcacaacaggctgatgtgcaagtaattaatacctataaggatattgttactagtccagttatcttgaaagatgcttctaagtggattaagaatccaactgaagtagtaaaacctgctaagaaggctaaatataagactttagctgatggaactaagaaattagttagaccagctgagccagcagtaattagaagagcaggacgtggatacaacgttagtgctaaggaaatgcaaaaagcagtttctgtgactacacaacaacaatcacaggttttcacgattagtgcgaagagtaatgatccagaaaagtcacaagctattgcaaatgcagttgctcaaacttttaagaacaaaatcaagagtattatgaatgttaacaatgtcactattgtttctcctgcttctgttggagcaaagacattccctaagacaaccctctttactttagctggagttgttctgggcttaattattagtgttgctttaattatcttgcgtgactcatttaatactacagtaagggacgatgattacttgactaaagaattaggtttaaccaatttaggtcatgtatctcacttccacttatctaataagtttagtattaataataatgacaatagttttggtaaaaagaagcgtgtatag

SEQ ID NO: 13 - DNA, 774 bp, Lactobacillus johnsonii
  Lactobacillus johnsonii strain FI9785 gene number 1181 epsC,tyrosine-protein kinase, positions 1111981 1112761 reverse
atgggattgtttaatagacgtaaaaaacgtggtactgatgagaccatgcaatatggtgctaagttaattaccttagctaagccacaaaatccagttagtgaacaatttagaactgttaagactaatattgattttacaagtgttgatcatcagattaaggccttagcctttacttctgccaatattagtgaaggtaagtcgacggtgacagttaatacagctgtaactatggctcaatcagggaaaaaagtcttattaattgatgctgatcttcatagacctactctacatcaaacctttgatattccgaatagagtaggattgacaacaattttgacttcacattctaatgaagtagacatggcagatattgtaaaagaagatattattcctaatctttctattatgccagctggtcctattccacctaacccagcacaattattaggttctaacagaatgcgagcattcttaaatatggttaaagaacattatgatttagttgtcttagaccttgcacctgttcttgaagtttctgatactcaaatccttgctagcgaaatggacggggtagtattagttgttcgtcaaggtgttactcaaaaggccggtattgaacgtgcaattgaaatgcttaacttaactaagacgcacgttttagggtatgtaatgaatgacgtaagaactggtcccgatggatatggatatggttatggatacggctatggctatggatatggttatagccagaaaaaagatacagaaacgaagtaa

SEQ ID NO: 14 - DNA, 771 bp, Lactobacillus johnsonii
  Lactobacillus johnsonii strain FI9785 gene number 1180 epsD,protein-tyrosine-phosphate phosphohydrolase, positions11112111111981 reverse
atggtattagttgatattcactcacatatattgccaggaattgatgatggttctcctgatttagattcatcattaaggttagcggaagctgcagttgcggatggaattacgcatgcactaatgactccgcatactttgaatggaaaatatttaaatcataaaaatgatatcattaaggatacagctaagtttcaggaagaattaaataaacataatattccattaactgtctttccaagtcaagaagttcgtttaaatggaaatttaccgcaagctttagacgatgatgatatcttattctgtgatgaagatggcagatatatgttattagaatttccaagtgaagatgtaccaacttatgccaaagatatgacttttaagttgcttggtcgtggaattacaccaatcattgttcatccagaaagaaattcaggtatcttggctcatccagaaaaattacaagaattcatcgaacaaggatgtttaacacaaataactgctagttcatatataggtgtatttggtaaagaaattgaaaaattagctgatcagtttgtagaagctggacaagtagctacttttgcttccgatgcacatacgttgccaaaacgtgaaagtcgaatgcatgacgcttatgagaagttagaaaagacgcaaggattggatgttgctaatagctttaagcagaatgcgagaaacattatcaattctgataatgttaatttaaattggaaacctttaaagaaaaagaaacgtttctggatattttga

SEQ ID NO: 15 - DNA, 660 bp, Lactobacillus johnsonii
  Lactobacillus johnsonii strain FI9785 gene number 1179 epsE,putative undecaprenyl-phosphate galactosephosphotransferase, positions 11105221111181 reverse
atgatggcacaagaggttaaaaaggtaaaattggatccttcaaaagttaatggacgtgtaggataccgtgctataaaacgtggatttgacgttttgaccagtggattggcattaatattattgtctccattatttttaattttaattgtcttaattaaaagggaagatggtggcccagcattttattcacaaacaagaatcggaaaaaatggaaaaccatttaaaatgtggaagtttcgttcaatgattgtgaatgccgataaaatggtaaaacaattagaagaacaaaatgaaattgacggggcaatgtttaagatcaaagatgatcctcgagtaactaagattggtcatgtaattagaaaatatagtcttgatgagctaccacagttatggaatgtgttaaaaggtgatatgtctttagttggacctcgtccacctctaccaatggaagtagaagattatacaccatatgacaagttacgtttaactgttacacctggctgtactggtctttggcaagtaaccaagcgtaatgatgcagattttgatgaaatggtagaattagaccttgaatacatcaataatagtagtctatggtttgattttaagattttattcagaactgtcggtgttgtcattcatccgaatagtgcatattag

SEQ ID NO: 16 - DNA, 1134 bp, Lactobacillus johnsonii
  Lactobacillus johnsonii strain FI9785 gene number 1178, undefined product, positions 11093781110511 reverse
atggataaaagaattaaagttattcacgtggctgaagctgcgggcggtgtggaaagatatttagaggctcttcttaaatatagcgataagtgtaaaatagagaatattttagtgtgttctcaaaactatgattataaaaaattcgaatcactaactagtcgcgttattgtattaaaaatgtctcataatattgatccaatagctgatattaaagttgaaaaggctttaagaaatataataaaacgagagagaccggatattgtttatgctcattcgagtaaagcaggtgcttttgccagaatcgctgatataggattgaagaacaaagttatttacaatccacatgggtgggcatttaatatgcaacaatccacaaaaaagaagcaaatgtataaatgggttgaaaaaatttcagcctatttttgtgatcaaatagtttgtatttcggacgccgagcggctttctgctctgagagaaaaaatatgtaagcctaataagttacaggttatttacaatgggatcgattttgttgaactgaacaaagaatccaagcaaaatgtagatctgtcaattcctaaaaatagttatgttattggaatggtaggaagactcagcgaacaaaaagctcctgatatatttgttgaagctgctcatttaattaaagaacaaatcccaaatgcgttttttttaatggttggtgacggtccacttaagaaacaaattgagagacaaatagataaattaggtttaaaagaatctttttgtattactggatgggtagagaatccaactgcttatatgaaaaaaatggatataggattacttatttcaagatgggaaggatttggattagttataccagaatatatggcatctaatattccagttattgcctcgagagttgatgcaattcctaatcttattgaagatggcaaagatgggattttagtaaataaagatgattttaagtcaattgctgaaaatgtggtacggttaaaaacaaatcctgatttatacactaagttgaaattgcaggcaatgagaaaagtaaagaattttgatataaaacgagtagcttctgaaacggaaaatttatattttgttctatataatggtgaaatgaatgaaaattaa

SEQ ID NO: 17 - DNA, 1029 bp, Lactobacillus johnsonii
  Lactobacillus johnsonii strain FI9785 gene number 1177, glycosyltransferase, positions 11083601109388 reverse
atgaaaattaatattatagttcccgtctataacgcagaaaaatatctaaaacaaaacatagaatgtgttttaaatcaagactataaaaatataaatttaattcttgtagatgatggttcggatgataattcattttatatttgtaagaaatatgcgcaaatggatgacagaattattttagtgcatcaagataactcgggtgtatcagtagcgagaaataagggcttggaatatgtagatggtgaatattttacttttttagatgtggatgatttcattgataatgaatatgtagctaatgttgctaatacaatagaaaaagagaatccggatattgtattgacatctgttataaaagagtataaaaacaatcatttagtaaatgatctttttgaacaaaaaatagtaaaaattaatggaaaaaaactattgagaagattgattggtccaattaaaaatgaaattaatcatccaataaaattggaagatcttaatccagtatggggtaaattttataaaactagcaaatttagacaaataagattcgaaaaaaatttgaaccgatcggaggatttattgtttaatttaaatgcatttttcttagctgaaaattgtgtgtataatggcaattcttattatcattataatagaattaatgagacctcaaatgtttcaaactatgatccaaatttatttgaaaagtttaaatttgtgaatactaaaatagtagagtttatatatcacaaaaaattaaatacagaatttcaagaggcacttaataatagaataattgcaaatttgataacgttagctatcaattattttggtaacaatagtaaggatgccacaattaaattcaaaaaaattttaaattctgactattataagaaagcctttaaaaattttgattttggatatttaaattttaaatatagaacattttttaaattatgtgaatataataaaattggattattaaaggtaatagtaaaaagtgctattaaaggtaaaagatacatcaagcgataa

SEQ ID NO: 18 - DNA, 1020 bp, Lactobacillus johnsonii
  Lactobacillus johnsonii strain FI9785 gene number 1176, glycosyltransferase, positions 11073231108342 reverse
atgggattaaataatggcacaccagaaatttctataatagtaccagtctataatgtagaaaagtggatagaaaattgtattcgtagtgttatagctcaaactttcaataattgggaacttattttagtaaatgatggtagcttggacaatagtcaacaaatatgtgaaaagtatgcaaagaaatatagtaaaattcatttaatcaataaagaaaatggtgggttatctgatgcacgaaattttggcataaaaaaagctaagggaaagtatttagcatttgttgatggagatgactatatagatagcaattatttaagtaaattacatacagcaataattagttcaaatgcatcaattgcagtttgtggatacaaggaagtagataataagaaaaagatactttctttaaaacgtaccaatgatttttttgtacaaaaactagtcagtggccaaacttttttgaggatacttgctgaaaagaattgcacactatgtgtagtggcttggaataagctttataaaaaagatttatttaagaataattcatacaaaaaaggtcggttgcatgaagatgaatttataattgctcctttagtatacgatgtatcaaaaattgctcttgtagatgatgaattatataactatgttcaaagagcaggcagtatcatgtcttcaaaaatgacacaaaagaatttattagatgcaaatgatgcctttgttgagagacttaatttttataaatccaataaaaatatgaaactatttgacttaactgttcatcaatatctattatggattttatctgtaatgaaatatcctaaaaaagttctcactaaaaaaggaagaatgattttacagaataactttaaacaatattcaaagtcacagaaaaataaatctaatacaaatattttcaaatttcaaatgaaattgggagaaagaaatataaagttagcatgggtgattgcgtatataattcctcatggaattaatagagtaatgggaattattaagaagtaa

SEQ ID NO: 19 - DNA, 828 bp, Lactobacillus johnsonii
  Lactobacillus johnsonii strain FI9785 gene number 1175, glycosyltransferase, positions 11064831107310 reverse
atgtcaaatatacctaaagttataaattatttttggtttggtggaaacccattaccggaagaagctaaaaaatgtattgctagttggagtaaatttcttccggattataaaattaaacgatgggatgaaagtaattttgatttatcttcttgtgaatatatggaagaagcatataaagcaaaaaaatgggcttttgtcagtgattatgcgcggattgcagttttagcaaaacatggtggattatattttgatactgatatggaattaataaagcctattgatgatattattcaagaaggctcttttattaatttagaaaagatatttaatggtgaacaggcacccggaatgagtgccatgggcgttgttccgcatttagaattgtttgaaagcttagtaaatatatataaaaaaagacattttattttaaataatgggacctatgatgagacaccaattggaagctatgtgaataaaatattaatagaaaagagagtatccttctccgatcaagtgacaagatgggatgaaattaatttttatccagcacgcttctttagcccaatgacttttgaggatggacgattagcgttaaaagatgatacacgtgcaatccatcattacgctgcgagttggcatgatgatgaagaaaaaaaagcaactaaaaaagttcaaaagattactagattatttggtaaaaaaatagggagatcttcccaccgaataatatttggatggatttctataaaaaaaacatataaaagcaatggattagcacctactatcagttttattagaaaaaagctaaagagagaaagtaaagaatga

SEQ ID NO: 20 - DNA, 1050 bp, Lactobacillus johnsonii
  Lactobacillus johnsonii strain FI9785 gene number 1174, putative glycosyltransferase, positions 1105437 1106486 reverse
atgaaaaaataccaaatagttatgaaaacagcagcaggtcaaaacgcaggaagcaaggcacctaatgatgtagtaaaaattgcagagaaattgaattttgaaaaattatttgttaatgtccatcgaaatgaatctgctttagataaagtgaaacagcaaattgaatataaaagtaattggaagagtgtttactctaaaatagaatctaattctattttattacttcaggttccaatttatgtacaccagttatcaagaatacattttcttaaaaaaataaaatcccaaaaaaaagtaaaattaatatttgtagtacatgatgtagaagaactacgtgtagcttttaataataattttcaaaaaaaacaatttgaggatatgctaaagttggctgatgtgattgtagttcataatgaagtaatggccaatttttttgaaaaaaaaggattccccaaagaaaaaattgtaaatctaaaaatatttgattatttatataattttgatctaaataaaaaagtaatcttttctaaaaaagtgattattgccggaaacttagatgaaaagaagacagaatatttaaaaaagctagataaaattgatgcaaaatttgatttatacggaccaaattatgttaagaagaattctaataagattacgtataaaggagttgtgccggcaaacgaattaccaaacctactagatagtggatttggattaatttgggatggaaatagcatagagacttgtagtggatattttggaaattatttaaaatataataatcctcataaattatctttatacttgacagcagggttgcctgtttttatttggagcaaagctgctgaagcaaaatttgtagatgaaaatcatttaggatatacaatagattctttatccgatattccattaatactagaaagacttactttagctgactataatagattaattaaaaatgttcgactagtaggagaaaaaatatctagaggagattttatgacagttgcactgacggatgctataaataacataaaggaaataaactaa

SEQ ID NO: 21 - DNA, 1260 bp, Lactobacillus johnsonii
  Lactobacillus johnsonii strain FI9785 gene number 1173, oligosaccharide repeat unit polymerase, positions 11041781105437 reverse
atgggttatgaaagagcaaaagttgtagattttataaaaaataaatatttatataagtttgttacttttatactttcctttttattgattttaaatactaattcaatatggacaactattccgggtactaaggaaaagtttataaatattctatatttagttttaccagtatgtattttttttagtttacttagtgtcaagatagacagtataaaatttagaaatatattggtattaacaatattttttcttatatattttagtatttttctgattgtaccagttaataggggctcaatttcgttagggctaaaattactagtatcattcattagttttttgttatattttggcttatgtaccgatactaagaaatttcctctgatttttgaatattacattaactggatggttattattggaatagtttctcttgtactgtggatgttagtttcctgtatgaggatattgcaatttaattctagtatattatcggattggtcaacatttaatggttacgcagctcgaatatcttcatatcatggtatttattttgaaactcagtatttgaataatataccacgaaatagtgctatttttcctgaagcacctatggcatccttacattttcttgtagctttatctttgaattttttattttgtcaaggaaaatttagtaaagcaaaaaatttacttttaatcttagccataataagtacattatcttcaacagggtacattgggatagtattgctatttacttataaaatggtcttttataaatttaaaaataatacacttaattatttaaaatttttatggatagtagtatttcttatcggtagtattgtgatcgttaatttgattttttctcaaaaaatagggagcgaatcaggaaatatccgaaaagacgattatttagctgcttttaatgcatggaaaacaagcccaatctttggtgtaggcttagtttctgatactgtaaaaaactatatgtctttgtggaggagttacaatttaggatttagtaattccttgatggatgttctcgcgcatgggggtctatggtcattagttgtgtatgtaggagcgggactaaaagggattattagtaatttgaaaataaagaattttaatatgattatgtttgtagtaatgacattttatttatttgcaactactatatttactaatagttttttgattttggctgtttttatatggatagcaacaagtaaaccaactgaagaaaaaatttaa

SEQ ID NO: 22 - DNA, 1113 bp, Lactobacillus johnsonii
  Lactobacillus johnsonii strain FI9785 gene number 1172 glf, undefined product, positions 11030441104156 reverse
atgaattatttagtagtaggttcgggtctttttggggcagtatttgctcatgaagcagcaaaacgcggtaataaggttacggtaattgaacaaagaaatcacttggcaggaaatatttatactaaggaagttgatgggattcaagttcatcaatatggagcacatatttttcatacctcaaataaagaagtttgggattatgtacaacaatttgctgaatttaatcgatacacaaatgcaccggtagctaattattatggtaaaatgtacaatttgccatttaatatgaatacttttagtgaaatgtggggagtaagaactcctcaagaagctcttaaaaaaatcaatgaacagcgtcaagaaatggcaggaaaagagccacaaaatcttgaagaacaagctatttcgttgattggacgcgatatctatgaaaagctaattaaaggctacactgaaaaacaatggggtagaaaagcaactgaattaccagcattcattatcaaacgcttaccagttcgattaacttatgataataactacttcaacgatgattatcaaggaattcctaagggtggatacactcaaattgttgaaaaaatgcttgataatgaaaatattgaagttaagttaaatactgatttctttgataataaagatgaatatttaaaaaactttgataagattgtttacacgggaatgattgataaattttttaattataagttaggcgagctagaataccgttcacttcattttgaaactgaagaaaaagatgttgataattaccaaggtaatgcagtaattaattatactgatgcagaaactccatatactcgtgtaattgaacataaacattttgaatttggtaaaggcgataaaaataaaacaattatcacacgtgaatatccagctgattggaaacgtggagatgaaccatactatccagttaacaatgaaagaaataataccctgtataaacaatacagagatttagctgaaaaagaaaatgacaaagttatttttggcggtcgtttaggtcaatacaaatactataatatggatcaagtaattgcagctgctttagaatctgtggaaaatgaatttggtgaatag

SEQ ID NO: 23 - DNA, 1422 bp, Lactobacillus johnsonii
  Lactobacillus johnsonii strain FI9785 gene number 1171 epsU,oligosaccharide translocase, positions 11016171103038 reverse
atgaaggtcattaaaaattacctttacaatgttggatatcaagtattagcaataattgtaccgctaatcacatcagcctatgtcagtcgtgttttgcgtccagaaggtgttggtgcaaattcttttactaattccataatgcaatattttattttatttgcaaatatgggaattggatattatgggaatagacaaattgcttatgttagagaaaataaagatcaaatgtcgaaaactttttgggaaatccaaatagttaaaacaattatgactctttatgctttcgtagcatttgaaatctttatgatattttatactcgtcagcctgaatatatgtgggctcaatcgcttaatttgattgcaattgctttcgatatttcatggttttatgaaggtatagaagatttcaaagttacggttttgaaaaattcattggtaaaagtgttatcaatgattgccatttttatcttcataaaaggacctaatgacgtaactctgtatatcattgtacttgcattatcaacattaattggtaatttaacactgtggccaaatataagacgtgatcttaataaaatttcctttaagtttttaaatccgtggcagcattttttaccaatggcagaattgttcattccacaaattgcgacacaagtttatgttcagcttaataaaactatgcttggggtaatggtcaatgaaacggcttctggctactatcagtattcagataatttagttaagttgattctagctcttgtaacagcaacggggacggttatgctaccacacgttgcgaatgctgtttcgcatggtgatatgcataaagtaaataaaatgttgtataaatcttttgattttgtctctgctatttcatttccgatgatgtttggattagctgctatttcattgaatttagcacctaagtattatggaccaggatatggtccagtaggtccggcgatgatgatagaatccatcgtaattttaattattgcatggagtaatgtactaggagttcaatatcttttacccattcataagcaaaaggaatttacgtggtctgttacgctaggagcaattataaatttaattttaaatgttcctttaattaatttgtggggattggacggtgcaatgtggtccactgttttatcagaaatttcagttacattgtatcagttgtgggctgtaagaaagttacttaattttaaaaaattatttgctgactcatggaaatatttgatagcaagtattggaatgtttgtggtagtcttctttatgaattcattcctaaaaagtacatggataatgctagggatttcgatcgtggtaggtgtagtagtttatggagttttactcattgtatttcaatctaaaatactaaaaagcataaatgagatagttaaaaataaattttaa

SEQ ID NO: 24 - DNA, 645 bp, Lactobacillus johnsonii
  Lactobacillus johnsonii strain FI9785 gene number 1170, conserved hypothetical protein, positions 11009511101595 reverse
atgaaaaaagaattgatagagaaaattgataagataggcagatattttaaagatagtagggatataaaaagattagaaaataaagactttacaattttttctagtaattgtataggcggaattatgtatcataatttacatctaaaattcttatctcccacaataaatttatggatagaacctgctgattatgttgcaatgttgagggatccaaagaaatactttgttagtggaaaaatggtggaagtaaaggacagtactcttccatatccggtgggaagtatttatggtaaacgtatatatggtgaacattataaaaattttgaagagttaagttttaagtgggaccaacgagtacaaagaattaattggaataatatttatgtattctttattgaaagagatggggccacaatagaagatttaactaatttcgataccttaccatttaacaaagttgtatttactaaaaaggaatatccagaattaaaaaattccattgttcttccaaatacttttgataatgaaaaaaatgaagtgaataatctatgtggaaaaataaataggttttctaggttacgttatatagatgaatttgattatgttaacttttttaataacgggtcaattattttgaaaaagtga

SEQ ID NO: 25 - protein, 334 aa, Lactobacillus johnsonii
  Lactobacillus johnsonii strain FI9785 gene number 1183 epsA undefined product
MDHKNSDNELRHRSHHHRHHRRKKFWRIFWIVLGVFLAVDIIAVIIAWHNIHVATNNMYNPMSNEISDRKVSDTLKDKKPMSLLLLGTDTGEFGRSYKGRTDTIMMMVINPKTNKTTVVSLPRDMKVNLPGYPDYSPAKINAAYTYGGVDETVKTIKKYFNVPTDAYVMVNMGGLEKAIDQVGGVTVKSPLTFDYEGYHFTKDVTYHMNGKKALAFSRMRYDDPRGDYGRQERQRLVIMALLKSSISYKTVVNQAFLNSISKQTMTNLTFDNMVALAQNYRHATDNVTTDHAQGQGDWENGVAYESVSRAECQRISNKLRAALGLKPETLKTGE

SEQ ID NO: 26 - protein, 287 aa, Lactobacillus johnsonii
  Lactobacillus johnsonii strain FI9785 gene number 1182 epsB polymerization and chain length determination protein
VENSTKTENTIDLRRLWMLLRAHIWSIILWAIGLGAVGFVLAAFVVEPKYTSTTQILVNQKRNAVDAGQAYNAQQADVQVINTYKDIVTSPVILKDASKWIKNPTEVVKPAKKAKYKTLADGTKKLVRPAEPAVIRRAGRGYNVSAKEMQKAVSVTTQQQSQVFTISAKSNDPEKSQAIANAVAQTFKNKIKSIMNVNNVTIVSPASVGAKTFPKTTLFTLAGVVLGLIISVALIILRDSFNTTVRDDDYLTKELGLTNLGHVSHFHLSNKFSINNNDNSFGKKKRV

SEQ ID NO: 27 - protein, 257 aa, Lactobacillus johnsonii
  Lactobacillus johnsonii strain FI9785 gene number 1181 epsC tyrosine-protein kinase
MGLFNRRKKRGTDETMQYGAKLITLAKPQNPVSEQFRTVKTNIDFTSVDHQIKALAFTSANISEGKSTVTVNTAVTMAQSGKKVLLIDADLHRPTLHQTFDIPNRVGLTTILTSHSNEVDMADIVKEDIIPNLSIMPAGPIPPNPAQLLGSNRMRAFLNMVKEHYDLVVLDLAPVLEVSDTQILASEMDGVVLVVRQGVTQKAGIERAIEMLNLTKTHVLGYVMNDVRTGPDGYGYGYGYGYGYGYGYSQKKDTETK

SEQ ID NO: 28 - protein, 256 aa, Lactobacillus johnsonii
  Lactobacillus johnsonii strain FI9785 gene number 1180 epsD protein-tyrosine-phosphate phosphohydrolase
MVLVDIHSHILPGIDDGSPDLDSSLRLAEAAVADGITHALMTPHTLNGKYLNHKNDIIKDTAKFQEELNKHNIPLTVFPSQEVRLNGNLPQALDDDDILFCDEDGRYMLLEFPSEDVPTYAKDMTFKLLGRGITPIIVHPERNSGILAHPEKLQEFIEQGCLTQITASSYIGVFGKEIEKLADQFVEAGQVATFASDAHTLPKRESRMHDAYEKLEKTQGLDVANSFKQNARNIINSDNVNLNWKPLKKKKRFWIF

SEQ ID NO: 29 - protein, 219 aa, Lactobacillus johnsonii
  Lactobacillus johnsonii strain FI9785 gene number 1179 epsE putative undecaprenyl-phosphate galactosephosphotransferase
MMAQEVKKVKLDPSKVNGRVGYRAIKRGFDVLTSGLALILLSPLFLILIVLIKREDGGPAFYSQTRIGKNGKPFKMWKFRSMIVNADKMVKQLEEQNEIDGAMFKIKDDPRVTKIGHVIRKYSLDELPQLWNVLKGDMSLVGPRPPLPMEVEDYTPYDKLRLTVTPGCTGLWQVTKRNDADFDEMVELDLEYINNSSLWFDFKILFRTVGVVIHPNSAY

SEQ ID NO: 30 - protein, 377 aa, Lactobacillus johnsonii
  Lactobacillus johnsonii strain FI9785 gene number 1178 undefined product
MDKRIKVIHVAEAAGGVERYLEALLKYSDKCKIENILVCSQNYDYKKFESLTSRVIVLKMSHNIDPIADIKVEKALRNIIKRERPDIVYAHSSKAGAFARIADIGLKNKVIYNPHGWAFNMQQSTKKKQMYKWVEKISAYFCDQIVCISDAERLSALREKICKPNKLQVIYNGIDFVELNKESKQNVDLSIPKNSYVIGMVGRLSEQKAPDIFVEAAHLIKEQIPNAFFLMVGDGPLKKQIERQIDKLGLKESFCITGWVENPTAYMKKMDIGLLISRWEGFGLVIPEYMASNIPVIASRVDAIPNLIEDGKDGILVNKDDFKSIAENVVRLKTNPDLYTKLKLQAMRKVKNFDIKRVASETENLYFVLYNGEMNEN

SEQ ID NO: 31 - protein, 342 aa, Lactobacillus johnsonii
  Lactobacillus johnsonii strain FI9785 gene number 1177 glycosyltransferase
MKINIIVPVYNAEKYLKQNIECVLNQDYKNINLILVDDGSDDNSFYICKKYAQMDDRIILVHQDNSGVSVARNKGLEYVDGEYFTFLDVDDFIDNEYVANVANTIEKENPDIVLTSVIKEYKNNHLVNDLFEQKIVKINGKKLLRRLIGPIKNEINHPIKLEDLNPVWGKFYKTSKFRQIRFEKNLNRSEDLLFNLNAFFLAENCVYNGNSYYHYNRINETSNVSNYDPNLFEKFKFVNTKIVEFIYHKKLNTEFQEALNNRIIANLITLAINYFGNNSKDATIKFKKILNSDYYKKAFKNFDFGYLNFKYRTFFKLCEYNKIGLLKVIVKSAIKGKRYIKR

SEQ ID NO: 32 - protein, 339 aa, Lactobacillus johnsonii
  Lactobacillus johnsonii strain FI9785 gene number 1176 glycosyltransferase
MGLNNGTPEISIIVPVYNVEKWIENCIRSVIAQTFNNWELILVNDGSLDNSQQICEKYAKKYSKIHLINKENGGLSDARNFGIKKAKGKYLAFVDGDDYIDSNYLSKLHTAIISSNASIAVCGYKEVDNKKKILSLKRTNDFFVQKLVSGQTFLRILAEKNCTLCVVAWNKLYKKDLFKNNSYKKGRLHEDEFIIAPLVYDVSKIALVDDELYNYVQRAGSIMSSKMTQKNLLDANDAFVERLNFYKSNKNMKLFDLTVHQYLLWILSVMKYPKKVLTKKGRMILQNNFKQYSKSQKNKSNTNIFKFQMKLGERNIKLAWVIAYIIPHGINRVMGIIKK

SEQ ID NO: 33 - protein, 275 aa, Lactobacillus johnsonii
  Lactobacillus johnsonii strain FI9785 gene number 1175 glycosyltransferase
MSNIPKVINYFWFGGNPLPEEAKKCIASWSKFLPDYKIKRWDESNFDLSSCEYMEEAYKAKKWAFVSDYARIAVLAKHGGLYFDTDMELIKPIDDIIQEGSFINLEKIFNGEQAPGMSAMGVVPHLELFESLVNIYKKRHFILNNGTYDETPIGSYVNKILIEKRVSFSDQVTRWDEINFYPARFFSPMTFEDGRLALKDDTRAIHHYAASWHDDEEKKATKKVQKITRLFGKKIGRSSHRIIFGWISIKKTYKSNGLAPTISFIRKKLKRESKE

SEQ ID NO: 34 - protein, 349 aa, Lactobacillus johnsonii
  Lactobacillus johnsonii strain FI9785 gene number 1174 putative glycosyltransferase
MKKYQIVMKTAAGQNAGSKAPNDVVKIAEKLNFEKLFVNVHRNESALDKVKQQIEYKSNWKSVYSKIESNSILLLQVPIYVHQLSRIHFLKKIKSQKKVKLIFVVHDVEELRVAFNNNFQKKQFEDMLKLADVIVVHNEVMANFFEKKGFPKEKIVNLKIFDYLYNFDLNKKVIFSKKVIIAGNLDEKKTEYLKKLDKIDAKFDLYGPNYVKKNSNKITYKGVVPANELPNLLDSGFGLIWDGNSIETCSGYFGNYLKYNNPHKLSLYLTAGLPVFIWSKAAEAKFVDENHLGYTIDSLSDIPLILERLTLADYNRLIKNVRLVGEKISRGDFMTVALTDAINNIKEIN

SEQ ID NO: 35 - protein, 419 aa, Lactobacillus johnsonii
  Lactobacillus johnsonii strain FI9785 gene number 1173 oligosaccharide repeat unit polymerase
MGYERAKVVDFIKNKYLYKFVTFILSFLLILNTNSIWTTIPGTKEKFINILYLVLPVCIFFSLLSVKIDSIKFRNILVLTIFFLIYFSIFLIVPVNRGSISLGLKLLVSFISFLLYFGLCTDTKKFPLIFEYYINWMVIIGIVSLVLWMLVSCMRILQFNSSILSDWSTFNGYAARISSYHGIYFETQYLNNIPRNSAIFPEAPMASLHFLVALSLNFLFCQGKFSKAKNLLLILAIISTLSSTGYIGIVLLFTYKMVFYKFKNNTLNYLKFLWIVVFLIGSIVIVNLIFSQKIGSESGNIRKDDYLAAFNAWKTSPIFGVGLVSDTVKNYMSLWRSYNLGFSNSLMDVLAHGGLWSLVVYVGAGLKGIISNLKIKNFNMIMFVVMTFYLFATTIFTNSFLILAVFIWIATSKPTEEKI

SEQ ID NO: 36 - protein, 370 aa, Lactobacillus johnsonii
  Lactobacillus johnsonii strain FI9785 gene number 1172 glf undefined product
MNYLVVGSGLFGAVFAHEAAKRGNKVTVIEQRNHLAGNIYTKEVDGIQVHQYGAHIFHTSNKEVWDYVQQFAEFNRYTNAPVANYYGKMYNLPFNMNTFSEMWGVRTPQEALKKINEQRQEMAGKEPQNLEEQAISLIGRDIYEKLIKGYTEKQWGRKATELPAFIIKRLPVRLTYDNNYFNDDYQGIPKGGYTQIVEKMLDNENIEVKLNTDFFDNKDEYLKNFDKIVYTGMIDKFFNYKLGELEYRSLHFETEEKDVDNYQGNAVINYTDAETPYTRVIEHKHFEFGKGDKNKTIITREYPADWKRGDEPYYPVNNERNNTLYKQYRDLAEKENDKVIFGGRLGQYKYYNMDQVIAAALESVENEFGE

SEQ ID NO: 37 - protein, 473 aa, Lactobacillus johnsonii
  Lactobacillus johnsonii strain FI9785 gene number 1171 epsU oligosaccharide translocase
MKVIKNYLYNVGYQVLAIIVPLITSAYVSRVLRPEGVGANSFTNSIMQYFILFANMGIGYYGNRQIAYVRENKDQMSKTFWEIQIVKTIMTLYAFVAFEIFMIFYTRQPEYMWAQSLNLIAIAFDISWFYEGIEDFKVTVLKNSLVKVLSMIAIFIFIKGPNDVTLYIIVLALSTLIGNLTLWPNIRRDLNKISFKFLNPWQHFLPMAELFIPQIATQVYVQLNKTMLGVMVNETASGYYQYSDNLVKLILALVTATGTVMLPHVANAVSHGDMHKVNKMLYKSFDFVSAISFPMMFGLAAISLNLAPKYYGPGYGPVGPAMMIESIVILIIAWSNVLGVQYLLPIHKQKEFTWSVTLGAIINLILNVPLINLWGLDGAMWSTVLSEISVTLYQLWAVRKLLNFKKLFADSWKYLIASIGMFVVVFFMNSFLKSTWIMLGISIVVGVVVYGVLLIVFQSKILKSINEIVKNKF

SEQ ID NO: 38 - protein, 214 aa, Lactobacillus johnsonii
  Lactobacillus johnsonii strain FI9785 gene number 1170 conserved hypothetical protein
MKKELIEKIDKIGRYFKDSRDIKRLENKDFTIFSSNCIGGIMYHNLHLKFLSPTINLWIEPADYVAMLRDPKKYFVSGKMVEVKDSTLPYPVGSIYGKRIYGEHYKNFEELSFKWDQRVQRINWNNIYVFFIERDGATIEDLTNFDTLPFNKVVFTKKEYPELKNSIVLPNTFDNEKNEVNNLCGKINRFSRLRYIDEFDYVNFFNNGSIILKK

SEQ ID NO: 39 - DNA, 25 bp, Artificial Sequence
  synthetic PCR amplification primer
tgcgcacccattagttcaacaaacg

SEQ ID NO: 40 - DNA, 25 bp, Artificial Sequence
  synthetic PCR amplification primer
ccaactaacggggcaggttagtgac

SEQ ID NO: 41 - DNA, 50 bp, Artificial Sequence
  synthetic annealing primer for translational-fusion linker
gatatcagaaaggaggttcagtccatggagtacttagatagctaagcgct

SEQ ID NO: 42 - DNA, 50 bp, Artificial Sequence
  synthetic annealing primer for translational-fusion linker
agcgcttagctatctaagtactccatggactgaacctcctttctgatatc

SEQ ID NO: 43 - DNA, 26 bp, Artificial Sequence
  synthetic PCR amplification primer for promoter region
agttcttagctcctatttttttgccc

SEQ ID NO: 44 - DNA, 33 bp, Artificial Sequence
  synthetic PCR amplification primer for promoter region
ttgataaattcgatttgaattatttgtttcgtc

SEQ ID NO: 45 - DNA, 24 bp, Artificial Sequence
  synthetic PCR amplification primer
atccatgggattgtttaatagacg

SEQ ID NO: 46 - DNA, 25 bp, Artificial Sequence
  synthetic PCR amplification primer
ttatttattacttcgtttctgtatc

(19) United States
(12) Patent Application Publication
(10) Pub. No.: US 2012/0225042 A1
(43) Pub. Date: Sep. 6, 2012

(54) COMPETITIVE EXCLUSION COMPOSITION AND METHODS

(75) Inventors: Arjan Narbad, Norwich (GB); Nicola Horn, Norwich (GB); Andrew Lee, Leicester (GB); Claire Shearman, Norwich (GB); Udo Wegmann, Norwich (GB)

(73) Assignee: Plant Bioscience Limited, Norfolk (GB)

(21) Appl. No.: 13/392,677

(22) PCT Filed: Aug. 25, 2010
(86) PCT No.: PCT/EP10/62423

§ 371 Date: May 2, 2012

(30) Foreign Application Priority Data
Aug. 26, 2009 (GB) 0914878.4

(51) Int. Cl. A61K 35/74; A61P 31/04; C07K 14/335; C12N 1/21; G01N 33/569; C07K 2/00; C12N 1/20; C12N 15/31

(52) U.S. Cl. 424/93.45; 435/252.9; 435/252.4; 536/23.7; 435/252.3; 435/7.1; 530/300; 530/350

(57) ABSTRACT

The present invention relates to compositions, including live organisms, nucleic acid and amino acid sequences, constructs and cells comprising these, which are used to competitively exclude pathogenic organisms from the alimentary canals of vertebrates to achieve prophylaxis or therapeutic effects.

### FIELD OF THE INVENTION

The present invention relates to compositions, including live organisms, nucleic acid and amino acid sequences, constructs and cells comprising these, which are used to competitively exclude pathogenic organisms from the alimentary canals of vertebrates to achieve prophylaxis or therapeutic effects.

### BACKGROUND OF THE INVENTION

Milner and Shaffer (1952) have observed that Salmonella infection in birds was suppressed with increasing age. Nurmi and Rantala (1973) were the first to suggest that this suppression was mediated by the development of the resident bacterial flora and proposed the concept of ‘competitive exclusion’. Experimental challenge studies have shown that the suppressive effect depends upon oral administration of viable bacteria, especially anaerobes (Schneitz and Mead 2000), and that undefined complex cultures are more suppressive than defined treatments (Impey et al. 1982; Stavric 1992). For poultry, agents that were derived directly from chicken intestines have been shown to be highly effective (Cameron and Carter 1992; Spencer et al. 1998) and a number of these undefined competitive exclusion agents are now commercially available (Collins and Gibson 1999; Rowland 1999). However, the risk exists that undefined agents may include pathogens and the use of such agents could result in their widespread transmission (Jin et al. 1998). Monoculture or well-defined multicultures could overcome this possible disadvantage (Reuter 2001).

Interventions in animal production aimed at the reduction of endemic and human food-borne pathogens include improved hygienic methods, vaccination and the use of antimicrobial agents. Whilst these methods are used widely for selected purposes, the regulatory pressures to reduce the use of a wide range of antibiotics in animal production (EC Council regulation 2821/98 1999) residues of which may remain in the meat and eggs have given rise to an increase of endemic diseases. One example in poultry production being a rise in necrotic enteritis caused by Clostridium perfringens (Ficken and Wages 1997; Van der Sluis 2000a,b). Thus, there is a drive for the development and use of alternative control approaches in production animals such as competitive exclusion, particularly using well-defined agents, that tackle both endemic disease and food-borne zoonoses (Reuter 2001).

U.S. Pat. No. 4,689,226 describes a process for the production of a bacterial preparation for the prophylaxis of intestinal disturbances especially Salmonella infections in poultry, made by anaerobically cultivating either separately or together bacterial strains of normal alimentary tract bacterial species, optionally in the presence of epithelial cells from the alimentary tract, for example the crop of a poult, and isolating the cultivated bacteria from the nutrient medium and making a preparation of them for instance by lyophilisation. The only bacterial strains used are those having an adhesion efficiency onto the epithelial cells of the alimentary tract of the poult of at least 10 bacteria per epithelial cell. However because these preparations include undefined agents they may include harmful pathogens and the use of such agents could result in widespread transmission.

The method comprises feeding to poultry an effective amount of a bacterial preparation consisting of four anaerobically co-cultured strains of normal alimentary tract bacterial species from poultry.

U.S. Pat. No. 4,839,281 describes a biologically pure culture of a strain of a Lactobacillus species in which the bacteria have avid adherence to intestinal cells, are able to survive at low pH and produce large amounts of lactic acid.

It also describes new L. acidophilus strains which render them beneficial to human health, and in particular render them useful in the treatment of the side effects of antibiotic therapy, ulcerative colitis and constipation; in providing resistance to gastrointestinal (GI) colonization by pathogenic microorganisms and yeast; in reducing fecal cholesterol excretion; and in reducing fecal estrogen excretion.

Members of the genus Lactobacillus constitute a diverse group of organisms, some of which are permanent members of the colonic commensal microflora (Kullen and Klaenhammer 1999).

Lactobacillus tend to be more prevalent in the small intestine than the colon in humans and many other animals.

Members of this genus have been used widely as competitive exclusion agents and, because it has been suggested that they confer other benefits to the host, they have also been defined as probiotics (Kasper 1998; Reid and Burton 2002).

Lactobacillus johnsonii is one of the many microorganisms that reside in the GI tracts of humans and animals such as poulty. Like all species of the Lactobacillus genus, it is an anaerobic, Gram-positive bacterium, which has a rod-like shape and does not undergo spore formation (Falsen, E., Pascual C., Sjoden B., Ohlen M., and Collins M. D. “Phenotypic and phylogenetic characterization of a novel Lactobacillus species from human sources: description of Lactobacillus iners sp. nov.” International Journal of Systemic Bacteriology. 1999. Volume 49. p. 217-221).

The human GI tract in which L. johnsonii resides is abundant with nutrients and relies upon more than 1000 microbial species that inhabit it in order to develop and function properly (Rajilic-Stojanovic M et al 2007 Env microbiol 9, 2125-2136). Specifically L. johnsonii and other GI tract microbes aid in polysaccharide and protein digestion and also generate a variety of nutrients, including vitamins and short-chain fatty acids that make up 15% of a human's total caloric intake. In addition, because L. johnsonii is able to undergo fermentation and can therefore make lactic acid, it plays a major role in the fermentation and preservation of various food items, such as dairy, meat, vegetable products, and cereal (Falsen, E., Pascual C., Sjoden B., Ohlen M., and Collins M. D. “Phenotypic and phylogenetic characterization of a novel Lactobacillus species from human sources: description of Lactobacillus iners sp. nov.” International Journal of Systemic Bacteriology. 1999. Volume 49. p. 217-221 and Pridmore, R. D., Berger, B., Desiere, F., Vilanova, D., Barretto, C., Pittet, A. C., Zwahlen, M. C., Rouvet, M., Alternann, E., Barrangou, R., Mollet, B., Mercenier, A., Klaenhammer, T., Arigoni, F., and Schell, M. A. “The genome sequence of the probiotic intestinal bacterium Lactobacillus johnsonii NCC 533.” Proceedings of the National Academy of Sciences of the United States of America. 2004. Volume 101. p. 2512-2517).

Finally, L. johnsonii is characterized as being part of the “acidophilus complex” of the Lactobacillus genus. This complex is comprised of six Lactobacillus species that are thought to be involved in probiotic activities, meaning they are able to undergo processes that are thought to be beneficial to human general health and well-being (Pridmore, R. D., Berger, B., Desiere, F., Vilanova, D., Barretto, C., Pittet, A. C., Zwahlen, M. C., Rouvet, M., Alternann, E., Barrangou, R., Mollet, B., Mercenier, A., Klaenhammer, T., Arigoni, F., and Schell, M. A. “The genome sequence of the probiotic intestinal bacterium Lactobacillus johnsonii NCC 533.” Proceedings of the National Academy of Sciences of the United States of America. 2004. Volume 101. p. 2512-2517 and Klaenhammer, T. R., Azcarate-Peril, M. A., Alternann, E., and Barrangou, R. “Influence of the Dairy Environment on Gene Expression and Substrate Utilization in Lactic Acid Bacteria.” The Journal of Nutrition. 2007. Volume 137. p. 748S-750S.

Such probiotic benefits particularly attributed to L. johnsonii include immunomodulation, pathogen inhibition, and epithelial cell attachment.

The genome of L. johnsonii strain NCC 533 was sequenced by the Nestle Research Center in Switzerland through the method of shotgun sequencing. The 1,992,676 base pair genome has a circular topology and is composed of 1,821 protein coding genes with 79 tRNAs (Pridmore, R. D., Berger, B., Desiere, F., Vilanova, D., Barretto, C., Pittet, A. C., Zwahlen, M. C., Rouvet, M., Alternann, E., Barrangou, R., Mollet, B., Mercenier, A., Klaenhammer, T., Arigoni, F., and Schell, M. A. “The genome sequence of the probiotic intestinal bacterium Lactobacillus johnsonii NCC 533.” Proceedings of the National Academy of Sciences of the United States of America. 2004. Volume 101. p. 2512-2517, Comprehensive Microbial Resource. Lactobacillus johnsonii NCC 533 Genome Page, and National Center for Biotechnology Information (NCBI) Genome. Lactobacillus johnsonii NCC 533, complete genome).

L. gasseri ATCC33323 is closely related to L. acidophilus NCFM which is slightly less homologous.

Many experts now believe that L. gasseri and L. johnsonii cannot be separated.

The Lactobacillus genus as a whole is characterized by its low Guanine+Cytosine content. L. johnsonii, in particular, contains a G+C content of 34.6% (Pridmore, R. D., Berger, B., Desiere, F., Vilanova, D., Barretto, C., Pittet, A. C., Zwahlen, M. C., Rouvet, M., Alternann, E., Barrangou, R., Mollet, B., Mercenier, A., Klaenhammer, T., Arigoni, F., and Schell, M. A. “The genome sequence of the probiotic intestinal bacterium Lactobacillus johnsonii NCC 533.” Proceedings of the National Academy of Sciences of the United States of America. 2004. Volume 101. p. 2512-2517).

Interestingly, L. johnsonii contains no genes which encode for the biosynthetic pathways necessary to generate amino acids and necessary cofactors. Rather, the genome encodes many amino acid proteases, peptidases, and phosphotransferase transporters and hence requires amino acids and peptides that come from its environment. In addition, genome sequencing has revealed that L. johnsonii contains all of the genes necessary for the synthesis of pyrimidines, but lacks genes necessary for the synthesis of purines. Thus, L. johnsonii also must depend on its environment in order to acquire purine nucleotides. Since this organism must obtain amino acids and purine nucleotides from exogenous sources, it is thought that it relies on its human host or other intestinal microorganisms in order to obtain such monomeric nutrients Pridmore, R. D., Berger, B., Desiere, F., Vilanova, D., Barretto, C., Pittet, A. C., Zwahlen, M. C., Rouvet, M., Alternann, E., Barrangou, R., Mollet, B., Mercenier, A., Klaenhammer, T., Arigoni, F., and Schell, M. A. “The genome sequence of the probiotic intestinal bacterium Lactobacillus johnsonii NCC 533.” Proceedings of the National Academy of Sciences of the United States of America. 2004. Volume 101. p. 2512-2517).

The cell surface of L. johnsonii contains various types of cell-surface proteins which are important in helping the microorganism attach to the mucosal surfaces of the GI tract. In addition, these cell-surface proteins can play a role in stimulating immune cells and can thus be one of the mechanistic explanations underlying the probiotic property of immunomodulation often attributed to L. johnsonii. Examples of these cell-surface proteins include mucus-binding proteins, glycosylated fimbriae, and an IgA protease (Pridmore, R. D., Berger, B., Desiere, F., Vilanova, D., Barretto, C., Pittet, A. C., Zwahlen, M. C., Rouvet, M., Alternann, E., Barrangou, R., Mollet, B., Mercenier, A., Klaenhammer, T., Arigoni, F., and Schell, M. A. “The genome sequence of the probiotic intestinal bacterium Lactobacillus johnsonii NCC 533.” Proceedings of the National Academy of Sciences of the United States of America. 2004. Volume 101. p. 2512-2517).

As an anaerobic, lactic acid producing bacterium, L. johnsonii obtains its energy by fermenting disaccharides and hexoses to lactic acid. Specifically, the sugars it uses as substrates include galactose, maltose, sorbose/sorbitol, gentiobiose, isoprimerevose, isomaltose, and panose. L. johnsonii's ability to undergo fermentation and thus produce lactic acid makes it a widely used microorganism in the industrial fermentation of dairy, meat, and vegetable products (Klaenhammer, T. R., Azcarate-Peril, M. A., Alternann, E., and Barrangou, R. “Influence of the Dairy Environment on Gene Expression and Substrate Utilization in Lactic Acid Bacteria.” The Journal of Nutrition. 2007. Volume 137. p. 748S-750S).

However, as mentioned above, L. johnsonii lacks the biosynthetic pathways necessary for the generation of essential nutrients such as amino acids, purine nucleotides, and cofactors. Because of this, the genes which code for transporters in this microorganism are highly expressed and thus L. johnsonii contains a great number of certain transporters that are less frequent in other microorganisms. Specifically, it contains an abundance of AA-permease transporters and phosphotransferase (PTS)-type transporters. In addition, L. johnsonii has numerous proteinases, peptide transporters and peptidases in order to acquire nutrients from exogenous sources (Pridmore, R. D., Berger, B., Desiere, F., Vilanova, D., Barretto, C., Pittet, A. C., Zwahlen, M. C., Rouvet, M., Alternann, E., Barrangou, R., Mollet, B., Mercenier, A., Klaenhammer, T., Arigoni, F., and Schell, M. A. “The genome sequence of the probiotic intestinal bacterium Lactobacillus johnsonii NCC 533.” Proceedings of the National Academy of Sciences of the United States of America. 2004. Volume 101. p. 2512-2517)

Due to its metabolic limitations and reliance on exogenous sources for nutrients, L. johnsonii is typically found in human and animal GI tracts where it can obtain nutrients from its host. As an auxotrophic bacterium that lacks certain enzymes needed for the digestion of complex carbohydrates, it is unable to compete with other GI tract bacteria such as Bifidobacteria, which inhabit the colon. Therefore L. johnsonii resides in the upper GI tract, which is rich in amino acids and peptides. Specifically, it is one of the dominant microorganisms found at the junction between the ileum of the small intestine and the cecum of the colon Pridmore, R. D., Berger, B., Desiere, F., Vilanova, D., Barretto, C., Pittet, A. C., Zwahlen, M. C., Rouvet, M., Alternann, E., Barrangou, R., Mollet, B., Mercenier, A., Klaenhammer, T., Arigoni, F., and Schell, M. A. “The genome sequence of the probiotic intestinal bacterium Lactobacillus johnsonii NCC 533.” Proceedings of the National Academy of Sciences of the United States of America. 2004. Volume 101. p. 2512-2517).

Other species within the Lactobacillus genus can be found in food, vegetation, sewage, and various areas of the human body. In humans, Lactobacillus species can be found in the intestine, oral cavity, and the vagina (Falsen, E., Pascual C., Sjoden B., Ohlen M., and Collins M. D. “Phenotypic and phylogenetic characterization of a novel Lactobacillus species from human sources: description of Lactobacillus iners sp. nov.” International Journal of Systemic Bacteriology. 1999. Volume 49. p. 217-221).

L. johnsonii, in particular, has many genes and transporters that allow it to release bile salt hydrolase, an important enzyme that is characteristic of microorganisms that live in the GI tract. Since L. johnsonii devotes many genes for the encoding of bile salt hydrolase, its importance and ability to compete and survive in its ecosystem can be correlated with its ability to produce such large amounts of this essential enzyme.

In addition, lactic acid bacteria, such as L. johnsonii are able to produce bacteriocins which have antibacterial properties that lactic acid bacteria can use against other microorganisms, thus providing them with ways to survive in their ecosystem. For example, L. johnsonii VPI 11088 is able to produce Lactacin F, a bacteriocin which can kill other Lactobacillus species as well as Enterococcus species in the GI tract. Thus, this microbe is thought to use this bacteriocin as a way to compete in the microbe-rich environment in which it lives (Pridmore, R. D., Berger, B., Desiere, F., Vilanova, D., Barretto, C., Pittet, A. C., Zwahlen, M. C., Rouvet, M., Alternann, E., Barrangou, R., Mollet, B., Mercenier, A., Klaenhammer, T., Arigoni, F., and Schell, M. A. “The genome sequence of the probiotic intestinal bacterium Lactobacillus johnsonii NCC 533.” Proceedings of the National Academy of Sciences of the United States of America. 2004. Volume 101. p. 2512-2517, and 5. Abee, T., Klaenhammer, T. R., and Letellier, L. “Kinetic Studies of the Action of Lactacin F, a Bacteriocin Produced by Lactobacillus johnsonii That Forms Poration Complexes in the Cytoplasmic Membrane.” Applied and Environmental Microbiology. 1994. Volume 60. p. 1006-1013).

Some lactic acid bacteria have been shown to use quorum sensing as a regulator for the expression of genes involved in the production of bacteriocins. For example, some species of the Lactobacillus genus such as Lactobacillus sake have been shown to utilize quorum sensing as a means of regulating bacteriocin gene expression (Risoen, P. A., Brurberg, M. B., Eijsink, V. G., and Nes, I. F. “Functional analysis of promoters involved in quorum sensing-based regulation of bacteriocin production in Lactobacillus.” Molecular Microbiology. 2000. Volume 37. p. 619-628).

L. johnsonii is not known to be pathogenic to humans. On the contrary, it is shown to be a beneficial microorganism which resides in the human intestine and is characterized by various probiotic properties (Falsen, E., Pascual C., Sjoden B., Ohlen M., and Collins M. D. “Phenotypic and phylogenetic characterization of a novel Lactobacillus species from human sources: description of Lactobacillus iners sp. nov.” International Journal of Systemic Bacteriology. 1999. Volume 49. p. 217-221, Pridmore, R. D., Berger, B., Desiere, F., Vilanova, D., Barretto, C., Pittet, A. C., Zwahlen, M. C., Rouvet, M., Alternann, E., Barrangou, R., Mollet, B., Mercenier, A., Klaenhammer, T., Arigoni, F., and Schell, M. A. “The genome sequence of the probiotic intestinal bacterium Lactobacillus johnsonii NCC 533.” Proceedings of the National Academy of Sciences of the United States of America. 2004. Volume 101. p. 2512-2517 and Klaenhammer, T. R., Azcarate-Peril, M. A., Alternann, E., and Barrangou, R. “Influence of the Dairy Environment on Gene Expression and Substrate Utilization in Lactic Acid Bacteria.” The Journal of Nutrition. 2007. Volume 137. p. 748S-750S).

Other species of the Lactobacillus genus are also known to be non-pathogenic. There have, however, been a small number of incidents where Lactobacillus has been a pathogen, but these few cases have involved people with previous diseases (Falsen, E., Pascual C., Sjoden B., Ohlen M., and Collins M. D. “Phenotypic and phylogenetic characterization of a novel Lactobacillus species from human sources: description of Lactobacillus iners sp. nov.” International Journal of Systemic Bacteriology. 1999. Volume 49. p. 217-221).

Lactobacillus johnsonii is able to undergo fermentation and produce lactic acid. This biochemical compound produced by L. johnsonii and other lactic acid bacteria provides the sour taste and texture along with a preservative effect for many consumed foods, especially milk and dairy products. For this reason, Lactobacillus and other lactic acid bacteria are commonly used in the industrial production of dairy products where they can be used as starter cultures necessary to generate products such as yogurt. They can also be introduced into food products for their probiotic effects (Klaenhammer, T. R., Azcarate-Peril, M. A., Alternann, E., and Barrangou, R. “Influence of the Dairy Environment on Gene Expression and Substrate Utilization in Lactic Acid Bacteria.” The Journal of Nutrition. 2007. Volume 137. p. 748S-750S).

For example, the presence of L. johnsonii in milk can help thicken mucous membranes and reduce the risk of developing stomach ulcers caused by Helicobacter pylori (Pantoflickova, D., Corthesy-Theulaz, I., Dorta, G., Stolte, M., Isler, P., Rochat, F., Enslen, M., Blum, A. L. “Favourable effect of regular intake of fermented milk containing Lactobacillus johnsonii on Helicobacter pylori associated gastritis.” Alimentary Pharmacology & Therapeutics. 2003. Volume 18. p. 805-813).

The effect of Lactobacillus on H. pylori has been shown to be greater when the Lactobacillus species is present in a cultured form such as milk (Hamilton-Miller, J. M. “The role of probiotics in the treatment and prevention of Helicobacter pylori infection.”International Journal of Antimicrobial Agents. 2003. Volume 22. p. 360-366).

Thus, such results indicate the possible further incorporation of the Lactobacillus species and other lactic acid bacteria in the industrial production of dairy products for their beneficial use as prophylaxis.

A large number of documents have described the addition of L. johnsonii to foods for a variety of reasons.

U.S. Pat. No. 5,603,930 described Lactobacillus johnsonii CNCM I-1225, as a strain which adheres to Caco-2 cells and inhibits adhesion thereto by enterovirulent and enteroinvasive pathogens. It also describes food compositions comprising this strain. The strain in question is particularly intended for administration to human beings or animals for therapeutic or prophylactic treatment of the GI system, more particularly as an antidiarrhoeic.

U.S. Pat. No. 6,022,568 disclosed an ice cream with coating containing lactic acid bacteria, including L. johnsonii.

U.S. Pat. Nos. 6,110,725 and 6,258,587 described recombinant sequence modified L. johnsonii which produce only L(+) lactate.

U.S. Pat. No. 6,410,016 described a method for administering viable microorganism compositions to poultry comprising L. reuteri and L. johnsonii.

U.S. Pat. No. 7,217,414 described methods of preventing peritonitis by administering lactic acid bacteria, including L. johnsonii CNCM 1-1225.

In addition, a study conducted by La Ragione et al. (2004) addressed the beneficial use of L. johnsonii in the poultry industry. This study found that the administration of L. johnsonii in chickens helped control diseases caused by Escherichia coli and Clostridium perfringens. Thus, L. johnsonii has the potential to be directly used in the poultry industry as an alternative to antimicrobials (La Ragione, R. M., Narbad, A., Gasson M. J., Woodward M. J. “In vivo characterization of Lactobacillus johnsonii FI9785 for use as a defined competitive agent against bacterial pathogens in poultry.” Letters in Applied Microbiology. 2004. Volume 38. p. 197-205).

Furthermore, as a probiotic bacterium with many potential benefits for human health, Lactobacillus johnsonii has been the subject of various research investigations, many of which show the potential of L. johnsonii as a treatment option for various human diseases. For example, one study by Kaburagi et al (2007) examined the effect of ingested Lactobacillus johnsonii in the diet of aged mice with Protein-Energy Malnutrition (PEM). PEM is an immune deficiency commonly seen in the human elderly population due to nutritional problems. In experiments done on aged mice through experiment-induced protein-energy malnutrition, the researchers were able to identify several immune system benefits associated with the inclusion of L. johnsonii La1 in the diet. Specifically, L. johnsonii La1 was able to positively influence both the intestinal and systemic immune system by partially restoring the number of serum IgA, IgG, and CD8+ cells, and enhancing the formation of splenocytes; all of which had decreased as a result of a low protein diet leading to PEM (Kaburagi, T., Yamano, T., Fukushima Y., Yoshima, H., Mito, N., and Sato, K. “Effect of Lactobacillus johnsonii La1 on immune function and serum albumin in aged and malnourished aged mice.” Nutrition. 2007. Volume 23. p. 342-350).

Another study by Inoue et al (2007) investigated the influence of L. johnsonii on immune system responses associated with Atopic Dermatitis, an inflammatory dermatological disease. In a comparison of the expression of genes involved in Atopic Dermatitis, the investigators found that while a control group of mice showed increased cytokine and CD86 levels following induction of a skin lesion, mice which had been orally administered L. johnsonii showed no elevation in cytokine or CD86 levels (Inoue, R., Otsuka M., Nishio, A., and Ushida, K. “Primary administration of Lactobacillus johnsonii in weaning period suppresses the elevation of proinflammatory cytokines and CD86 gene expressions is skin lesions in NC/Nga mice.” FEMS Immunology & Medical Microbiology. 2007. Volume 50. p. 67-76).

Furthermore, L. johnsonii may even have a potential treatment role in the management of diabetes. One study by Yamano et al (2006) found that the oral administration of L. johnsonii reduced glucose and glucagon levels in diabetic rats which had been subject to intracranial injection of 2-deoxy-D-glucose (2DG). The investigators also present a possible mechanism by which L. johnsonii affects the autonomic nervous system and thus modulates an anti-diabetic response (Yamano, T., Tanida, M., Niijima, A., Maeda K., Okumura, N., Fukushima, Y., and Nagai, K. “Effects of the probiotic strain Lactobacillus johnsonii strain La1 on autonomic nerves and blood glucose in rats.” Life Science. 2006. Volume 79. p. 1963-1967).

In light of this background, those skilled in the art will appreciate that there is an ongoing need for new and improved compositions and methods for achieving competitive exclusion of pathogenic organisms.

Accordingly, it is an object of this invention to provide L. johnsonii FI9785 in an isolated form.

Another object of this invention is to provide L. johnsonii FI9785 in an isolated form for use as a competitive exclusion agent.

Another object of this invention is to provide L. johnsonii FI9785, nucleic acids isolated from L. johnsonii FI9785 amino acids isolated from L. johnsonii FI9785 or constructs or cells comprising any of these in an isolated form.

Another object of this invention is to provide L. johnsonii FI9785, nucleic acids isolated from L. johnsonii FI9785 amino acids isolated from L. johnsonii FI9785 or constructs or cells comprising any of these in an isolated form as a competitive exclusion agent.

Another object of this invention is to provide nucleic acid and amino acid compositions isolated from L. johnsonii FI9785 in an isolated form for identifying other organisms which may be used for competitive exclusion.

Other objects and advantages of this invention will be appreciated by those skilled in the art upon review of the complete disclosure provided herein and the appended claims, reference to which should be made for an appreciation of the full scope of the invention provided herein.

### BRIEF DESCRIPTION OF THE DRAWINGS

FIG. 1—Graphs showing shedding of Salmonella Enteritidis (S1400, nalr), Escherichia coli O78:k80 (EC34195 nalr) and Clostridium perfringens (FD00385) from specific pathogen-free chicks dosed singly (a, c and e) or following a Lactobacilli predose (b, d and f).

FIG. 2—SEQ ID 1 FI9785 gene number 111 nucleotide sequence.

FIG. 3—SEQ ID 2 FI9785 gene number 1070 nucleotide sequence.

FIG. 4—SEQ ID 3 FI9785 gene number 1481 nucleotide sequence.

FIG. 5—SEQ ID 4 FI9785 gene number 1482 nucleotide sequence.

FIG. 6—SEQ ID 5 FI9785 gene number 1651 nucleotide sequence.

FIG. 7—SEQ ID 6 FI9785 gene product of gene number 111 protein sequence.

FIG. 8—SEQ ID 7 FI9785 gene product of gene number 1070 protein sequence.

FIG. 9—SEQ ID 8 FI9785 gene product of gene number 1481 protein sequence.

FIG. 10—SEQ ID 9 FI9785 gene product of gene number 1482 protein sequence.

FIG. 11—SEQ ID 10 FI9785 gene product of gene number 1651 protein sequence.

FIG. 12—SEQ ID 11 FI9785 gene number 1183 epsA nucleotide sequence.

FIG. 13—SEQ ID 12 FI9785 gene number 1182 epsB nucleotide sequence.

FIG. 14—SEQ ID 13 FI9785 gene number 1181 epsC nucleotide sequence.

FIG. 15—SEQ ID 14 FI9785 gene number 1180 epsD nucleotide sequence.

FIG. 16—SEQ ID 15 FI9785 gene number 1179 epsE nucleotide sequence.

FIG. 17—SEQ ID 16 FI9785 gene number 1178 nucleotide sequence.

FIG. 18—SEQ ID 17 FI9785 gene number 1177 nucleotide sequence.

FIG. 19—SEQ ID 18 FI9785 gene number 1176 nucleotide sequence.

FIG. 20—SEQ ID 19 FI9785 gene number 1175 nucleotide sequence.

FIG. 21—SEQ ID 20 FI9785 gene number 1174 nucleotide sequence.

FIG. 22—SEQ ID 21 FI9785 gene number 1173 nucleotide sequence.

FIG. 23—SEQ ID 22 FI9785 gene number 1172 glf nucleotide sequence.

FIG. 24—SEQ ID 23 FI9785 gene number 1171 epsU nucleotide sequence.

FIG. 25—SEQ ID 24 FI9785 gene number 1170 nucleotide sequence.

FIG. 26—SEQ ID 25 FI9785 gene product of gene number 1183 EpsA protein sequence.

FIG. 27—SEQ ID 26 FI9785 gene product of gene number 1182 EpsB protein sequence.

FIG. 28—SEQ ID 27 FI9785 gene product of gene number 1181 EpsC protein sequence.

FIG. 29—SEQ ID 28 FI9785 gene product of gene number 1180 EpsD protein sequence.

FIG. 30—SEQ ID 29 FI9785 gene product of gene number 1179 EpsE protein sequence.

FIG. 31—SEQ ID 30 FI9785 gene product of gene number 1178 protein sequence.

FIG. 32—SEQ ID 31 FI9785 gene product of gene number 1177 protein sequence.

FIG. 33—SEQ ID 32 FI9785 gene product of gene number 1176 protein sequence.

FIG. 34—SEQ ID 33 FI9785 gene product of gene number 1175 protein sequence.

FIG. 35—SEQ ID 34 FI9785 gene product of gene number 1174 protein sequence.

FIG. 36—SEQ ID 35 FI9785 gene product of gene number 1173 protein sequence.

FIG. 37—SEQ ID 36 FI9785 gene product of gene number 1172 Glf protein sequence.

FIG. 38—SEQ ID 37 FI9785 gene product of gene number 1171 EpsU protein sequence.

FIG. 39—SEQ ID 38 FI9785 gene product of gene number 1170 protein sequence.

FIG. 40—Competition of E. coli with different strains of Lactobacilli in gut tissue explant assay. Adhesion of E. coli cells alone (orange bar) or in the presence of lactobacilli (blue bar)

FIG. 41—Shows the relative adhesion of Lactobacilli species to HEp-2 tissue cell line.

FIG. 42—Competitive exclusion of E. coli with Lactobacilli in an in vitro HEp-2 tissue cell line. Adhesion of E. coli cells alone (blue bar) or in the presence of Lactobacilli (purple bar)

FIG. 43—Shows the effect of predosing Lactobacillus johnsonii administration on the colonisation by Campylobacter jejuni. The control group (a) of birds were challenged with C. jejuni on day 2 and the data indicate the level of C. jejuni invasion in different tissues of the birds during the 35 day trial. The sample group (b) the birds were pre dosed with L. johnsonii on day 1 before challenge by C. jejuni.

FIG. 44—Shows the location of EPS related genes in the chromosome of Lactobacillus johnsonii FI9785. Arrows indicate the relative size of genes and the direction of transcription.

FIG. 45 Comparison of colony morphologies of parental strains after 24 h incubation on MRS at 37° C.

FIG. 46 Over-expression of EpsC in the Lb. johnsonii smooth colony variant FI10386; colonies on MRS_{Cm} agar after 24 h incubation at 37° C.

FIG. 47 2-D gel electrophoresis comparing cell-free protein extracts of Lb. johnsonii FI9785 and the smooth colony variant FI10386.

FIG. 48 A Magnified region of the 2-D gel in Fig XA illustrating the EpsC smooth protein spot (in blue) that is absent from the corresponding FI9785 sample. Sequencing the epsC gene of FI10386 identified a base change when compared to the FI9785 sequence, this results in a single amino acid substitution (aspartate to asparagine) at position 88 in the EpsC protein.

FIG. 49 Map of pFI2431

FIG. 50 Colonisation of L. johnsonii in the chicken caecum

FIG. 51 Colonisation of C. jejuni in the chicken caecum

FIG. 52 Oocyte excretion in each individual bird. During the experiment one bird from the control group had died.

FIG. 53 Average oocyst excretion in the two groups of birds treated with and without L. johnsonii

### SUMMARY OF THE INVENTION

Disclosed herein are genes, gene products, genetic constructs, methods of using such constructs and cells comprising such nucleic acids and constructs which have utility as CE agents.

The esp gene of L. johnsonii is shown to be crucial to the CE functionality of this organism and is likewise described, enabling the use of the eps gene and gene products to confer this function on other organisms, and enabling the use of compositions derived from this gene to identify other organisms exhibiting this functionality.

In a first aspect the invention relates to a culture of a Lactobacillus species or strain comprising a portion of the EPS gene cluster base sequence depicted in any one of SEQ ID NOS 11,12,13,14,15,16,17,18,19,20,21,22,23 and 24.

In a second aspect the invention relates to a culture of a Lactobacillus species or strain comprising a mucin binding protein having the nucleotide base sequence depicted in any one of SEQ ID NOS 1,2,3,4 and 5.

In a third aspect the invention relates to a culture wherein the Lactobacillus species is Lactobacillus johnsonii or Lactobacillus gasseri.

In a further aspect the invention relates to a culture wherein the Lactobacillus strain is L. johnsonii FI9785.

In a further aspect the invention relates to a culture wherein the Lactobacillus strain is deposited with NCIMB as deposit number NCIMB 41632.

In a further aspect the invention relates to a culture wherein the culture is a monoculture.

In a further aspect the invention relates to a culture wherein the culture is a mixed culture.

In a further aspect the invention relates to a method of restricting the colonisation of a vertebrate gut by one or more pathogens comprising administering a protectively effective amount of a composition comprising live L. johnsonii.

In a further aspect the invention relates to a method wherein the composition further comprises live B. subtilis.

In a further aspect the invention relates to a method wherein the pathogen is selected from C. perfringens, E. coli, and Campylobacter.

In a further aspect the invention relates to a method of prophylaxis against necrotis entiritis comprising administering a protectively effective amount of a composition comprising live.

In a further aspect the invention relates to a method of improving one or more of: the weight gain; feed conversion; and the immune competency of immature vertebrates comprising administering a composition comprising live L. johnsonii.

In a further aspect the invention relates to a food composition comprising a culture as hereinbefore described.

In a further aspect the invention relates to a method wherein the vertebrate is selected from the group consisting of: humans, bovine, ovine, porcine, equine, avian, pets and companion animals.

In a further aspect the invention relates to an isolated nucleic acid comprising a portion of the EPS gene cluster base sequence depicted in any one of SEQ ID NOS 11,12,13,14,15,16,17,18,19,20,21,22,23 and 24.

In a further aspect the invention relates to an isolated nucleic acid comprising a mucin binding protein having the nucleotide base sequence depicted in any one of SEQ ID NOS 1,2,3,4 and 5.

In a further aspect the invention relates to an isolated nucleic acid which encodes the protein or peptide sequence depicted in any one of SEQ ID NOS 6-10.

In a further aspect the invention relates to an isolated nucleic acid which encodes the protein or peptide sequence depicted in any one of SEQ ID NOS 25-38.

In a further aspect the invention relates to a method for conferring enhanced adhesion to a bacterium comprising introducing into said bacterium a nucleic acid as hereinbefore described.

In a further aspect the invention relates to a protein or peptide sequence comprising the amino-acid sequence depicted in any one of SEQ ID NOS 6-10.

In a further aspect the invention relates to a protein or peptide sequence comprising the amino-acid sequence depicted in any one of SEQ ID NOS 25-38.

In a further aspect the invention relates to a method of identifying a candidate organism for competitively excluding pathogens when introduced into the GI tract of a vertebrate: comprising hybridizing nucleic acids from said candidate organism to a nucleic acid sequence selected from the group consisting of SEQ ID NOS 1-5 and SEQ ID NOS 11-24.

In a further aspect the invention relates to a method of identifying a candidate organism for competitively excluding pathogens when introduced into the GI tract of a vertebrate: comprising raising antibodies to an immunogenic portion of a sequence selected from the group consisting of SEQ ID. 6-10 and SEQ ID. 25-38 and using the antibodies to identify candidate organisms which react with said antibodies.

In a further aspect the invention relates to a live culture as described herein for use as a coccidiostat.

In a further aspect the invention relates to a live culture as described herein for use as a probiotic.

### DETAILED DISCLOSURE OF THE INVENTION

In 2004, La Ragione et al., reported on the in vivo characterization of Lactobacillus johnsonii FI9785 for use as a defined competetive exclusion (CE) agent against bacterial pathogens in poultry.

La Ragione et al., showed that L. johnsonii FI9785, a poultry-derived isolate that adhered well to tissue culture and chick gut explant tissues out-competes challenge bacteria (La Ragione et al. 2002), and excludes a number of pathogens from poultry.

Using poultry models for Escherichia coli serotype O78:K80, a commensal and opportunistic pathogen of poultry, Salmonella enterica serotype Enteritidis, a human pathogen that colonizes poultry but without causing clinical signs and C. perfringens, the cause of necrotic enteritis in poultry (Allen-Vercoe, E and Woodward, M J (1999) Colonisation of the chicken caecum by afimbriate and aflagellate derivatives of Salmonella enterica serotype Enteridis. Veteerinary Microbiology 69, 265-275; Dibb-Fuller, M., Allen-Vercoe, E., Thorns, C. J. and Woodward, M. J. (1999) Characterisation of fimbrial and flagella mediated adherence to and invasion of INT407 monolayers by Salmonella enterica serotype Enteritidis. Microbiology 145, 1023-1131; La Ragione, R. M., Collighan, R. J. and Woodward, M. J. (1999) Noncurliation of Escherichia coli O78:K80 isolates associated with IS1 insertion in csgB and reduced persistence in poultry infection, FEMS Microbiology Letters 174, 247-253; La Ragione, R. M., Cooley, W. A. and Woodward, M. J. (2000a) Adherence of avian Escherichia coli O78:K80 to tissue culture, tracheal and gut explants; the role of fimbriae and flagella. Journal of Medical Microbiology 49, 327-338; La Ragione, R. M., Sayers, A. R. and Woodward, M. J. (2000b) The role of flagella and fimbriae in the colonisation, invasion and persistence of Escherichia coli O78:K80 in the day-old-chick model. Epidemiology and Infection 124, 351-363; La Ragione, R. M. and Woodward, M. J. (2003) CE by Bacillus subtilis spores of Salmonella enterica serotype Enteritidis and Clostridium perfringens in young chickens. Veterinary Microbiology, 94, 245-256); showed that predosing poultry with L. johnsonii FI9785 had an effect upon colonization and shedding of E. coli O78:K80, S. Enteritidis and C. perfringens.

L. johnsonii FI9785 (formerly NCIMB 30150) was deposited with NCIMB Ltd Ferguson Building, Craibstone Estate, Bucksburn, Aberdeen AB21 9YA, on 16 Apr. 2002 and given the accession no.: NCIMB 30150 as a safe or secure deposit. However it was not made publically available until 26 Jun. 2009 when strain number FI9785 was deposited with NCIMB as deposit number NCIMB41632 to comply with the requirements of the Budapest Treaty.

This disclosure describes in detail the isolation, characterization and use of commensal GI tract strains, with potential for pathogen exclusion.

The LAB strains, compositions and methods described herein have utility in food animals, companion animals, poultry, fish, horses and man, to exclude pathogens and contribute positive health benefits which are characterized and described herein.

It is known that poultry is a major reservoir of Salmonella and Campylobacter jejuni/coli the principle causative agent of food borne human disease. The biocontrol compositions and methods disclosed herein provide CE agents. These CE agents may be used in isolation or as part of an integrated approach involving antimicrobials and/or vaccines to reduce or eliminate food pathogens from poultry and animal hosts or gut pathogens such as Clostridium difficile from humans.

The strain of Lactobacillus johnsonii FI9785 disclosed herein has been shown both in vivo and in vitro to competitively exclude food-borne pathogens from the poultry GI tract which represents a major reservoir of these pathogens.

Using the intrinsic plasmids both cloning and expression vectors have been developed to establish a genetic tag for the strain. This allows further studies to understand the molecular mechanism of the CE process. This knowledge has been essential in the development of rational strategies for CE processes.

Natural animal isolates of Lactobacillus can be used to exclude pathogens before they enter the food chain. Lactobacillus johnsonii FI9785 has been shown to be especially effective in excluding pathogenic Clostridium perfringens from the GI tract of poultry which represents a major reservoir of human food-borne pathogens.

The use of antibiotics in animal production is increasingly restricted by legislation both in the UK and the EU in general. This will inevitably result in increased levels of pathogens in a range of different animal hosts; accordingly viable alternatives to antibiotics are required.

The use of CE is one promising approach. A detailed understanding of the mode or modes of action of CE treatment facilitates progress in the development of these treatments in a rational, scientific way.

The molecular tools disclosed herein provide a means to achieve such mechanistic information. This knowledge, in combination with improvements in antimicrobials and vaccine delivery systems, permits the development of effective CE strategies for controling the spread of human food borne pathogens in the food chain.

A number of complementary approaches, including molecular genetics, proteomics and functional genomics permit the utilisation of the Lactobacillus johnsonii FI9785 strain to harness and isolate its probiotic properties. They have also helped elucidate the mechanism by which Lactobacillus johnsonii FI9785 strain adheres and colonises the poultry GI tract.

Genome sequencing of this strain has made it possible to prepare DNA microarrays that are useful in functional genomics to better understand the gene expression within the GI tract environment. The effects of administration of this strain on the gut health and immune system of the host are also thereby made accessible.

The Lactobacillus johnsonii strain FI9785 described herein facilitates protection against necrotic entiritis. This is a debilitating disease caused by C. perfringens and results in the loss of millions of birds each year which has a subsequent huge economic impact.

It is disclosed herein that Lactobacillus johnsonii FI9785 may be given to poultry for use as a CE agent to control C. perfringens.

It is also disclosed that Lactobacillus johnsonii FI9785 is a valuable tool for controlling the endemic disease of necrotic enteritis, thereby reducing economic losses associated with reduced use of antimicrobials in the poultry industry.

We provide evidence herein that administration of FI9785 resulted in protection against necrotic entiritis in broiler chicken. Additional beneficial effects of the administration of FI9785 include improvements in weight gain and feed conversion, and improvements in the immune competency of young broiler chickens.

It is well known that at birth the immune system of newly hatched birds is underdeveloped and as the maternal antibody levels drop they become increasingly susceptible to pathogen colonisation. Thus FI9785 may have additional utility as a means of stimulating the immune system of immature animals such as young broiler chickens.

We also demonstrate herein the utility of FI9785 compositions to achieve exclusion of Campylobacter, a pathogen that is responsible for approximately 100,000 cases of food-borne poisoning in humans each year in the UK alone. FI9785, although not eliminating Campylobacter completely, brought about a marked reduction in their levels.

Without wishing to be limited to mechanistic considerations, it appears that CE operates by one or more of the following mechanisms:

(a) competition for receptor sites;
(b) antimicrobial production, such as VFA or bacteriocins;
(c) modulation of the gut flora;
(d) competition for essential nutrients.

Use of genetically tagged strains in conjunction with differential green fluorescent protein (GFP) expression strains of Salmonella and Campylobacter sp., allow for simultaneous identification, cellular localisation and monitoring of the distribution and prevalence of both the LAB culture and the challenged pathogen in the intestine.

Alternatively tagged strains can be used in isolation to investigate the interactions of one or more strain with one another and/or with the challenge pathogen in the intestine.

Intestinal adhesion capability is critical to the colonisation level of any bacterial strain. Both in vitro and in vivo, the adhesion colonisation capability of FI9785 and other LAB strains was studied using cell cultures derived from the epithelial cell lining of the large intestine and tissue explant fragments of different regions of the chicken GI tract, to assess the adherence of different protective strains of LAB.

In vivo adherence efficacy of the FI9785 strain was demonstrated by the administration of FI9785 to chicks. Their colonisation was demonstrated by both monitoring the shedding of the added strains and also by biopsy of the gut.

The molecular basis of the adherence mechanism was revealed by isolation of LAB strains defective in adherence (adh⁻) and analyzing the differences in protein expression profiles of adh⁺ and adh⁻ LAB. Using MOLDI-TOF the adhesins required for adherence were identified and excised using a Flexys® spot-excision robot, trypsin-digestion and analysed using a Bruker MALDI-ToF.

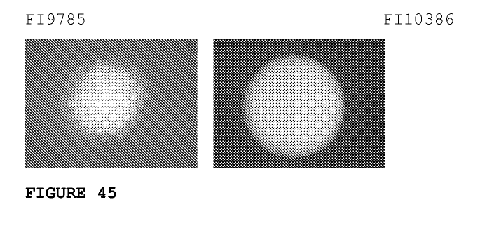

It was observed that there were two colony phenotypes present which we called rough (wt) and smooth (mutant) FI10386 (FIG. 45). Adhesion assays were performed and it was found that the smooth phenotype was less adherent. 2D protein analysis of the cell free extracts of both cell types were performed and it was found that an additional protein band was present in the smooth phenotype at a position on the gel where it was absent in the wt (figure XA, XB).

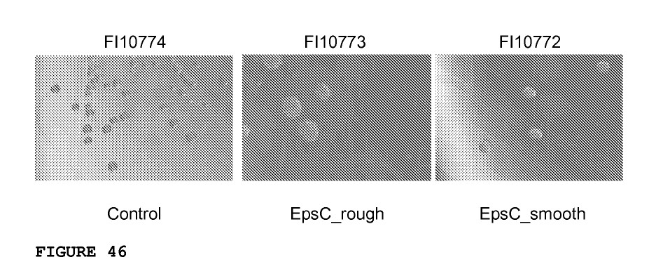

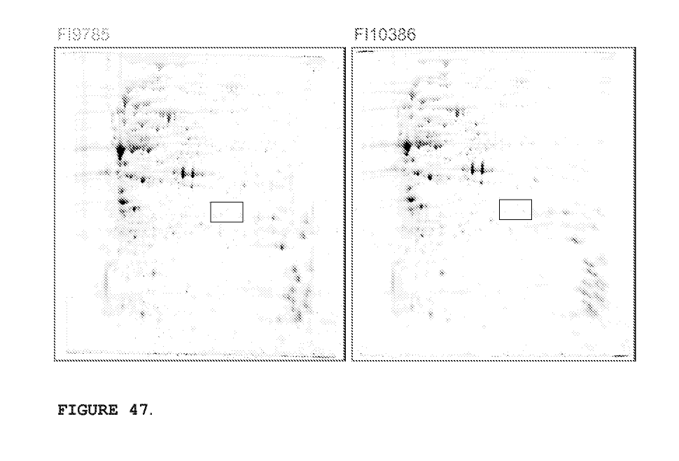

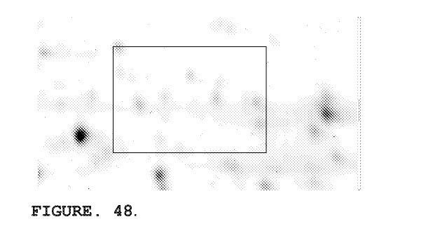

This protein was identified as a putative enzyme involved in exopolysaccharide synthesis and the gene was designated epsC. The gene for this protein was sequenced in both cell types and it was found that in the mutant there was a point mutation leading to a substitution. We theorised that this point mutation inactivates the enzyme. To prove the validity of this theory we subcloned and overexpresed both wt and mutated espC gene and overexpressed them in the smooth FI10386. The results indicated reversion to rough phenotype only with expression of wt type espC (FIG. 46) this complementation was also reflected in the adhesion phenotype.

### EXAMPLES

### Example 1

### In Vivo Use of the L. Johnsonii FI9785 to Achieve CE in Poultry

The methods described in La Ragione, R. M., Narbad, A., Gasson M. J., Woodward M. J. “In vivo characterization of Lactobacillus johnsonii FI9785 for use as a defined competitive agent against bacterial pathogens in poultry.” Letters in Applied Microbiology. 2004. Volume 38. p. 197-205 were used to test the efficacy of Lactobacillus johnsonii FI9785 in reducing the colonization and shedding of Salmonella enterica serotype Enteritidis, Escherichia coli O78:K80 and Clostridium perfringens in poultry.

Specific pathogen-free chicks (1 day old) were dosed with a single oral inoculum of 1×10⁹ colony forming units (CFU) Lactobacillus johnsonii FI9785 and 24 h later were challenged in separate experiments with S. Enteritidis (S1400, nalr) and E. coli O78:K80 (EC34195, nalr). There were no significant effects against S. Enteritidis whereas colonization of the small intestine by E. coli O78:K80 was reduced significantly. Both S. Enteritidis and E. coli colonized the caeca and colon to levels equivalent to control birds and there was no reduction in shedding as assessed by a semi-quantitative cloacal swabbing technique. Specific pathogen-free chicks (20 day old) were dosed with a single oral inoculum of 1×10⁹ CFU L. johnsonii FI9785 and 24 h later were challenged with C. perfringens. A single oral dose of L. johnsonii FI9785 was sufficient to suppress all aspects of colonization and persistence of C. perfringens.

Strains and Culture Conditions Used:

A nalidixic acid-resistant derivative of a S. Enteritidis wild-type (S1400 nalr described previously (Allen-Vercoe et al. 1999). A nalidixic acid-resistant derivative of a E. coli O78:K80 EC34195 described previously La Ragione, R. M., Sayers, A. R. and Woodward, M. J. (2000b) The role of flagella and fimbriae in the colonisation, invasion and persistence of Escherichia coli O78:K80 in the day-old-chick model. Epidemiology and Infection 124, 351-363.

Clostridium perfringens strain FD00385 was isolated from a clinical case of avian necrotic enteritis submitted to the Veterinary Laboratories Agency for routine diagnosis. Lactobacillus johnsonii (FI9785) was obtained from the culture collection at Institute of Food Research.

Salmonella Enteritidis nalr, E. coli O78:K80 nalr, C. perfringens, L. johnsonii (FI9785) were each stored separately, frozen at −80° C. in heart infusion broth supplemented with glycerol (30% w/v). Working cultures of the pathogenic cultures were grown on blood agar and stored at 4° C. as described previously La Ragione, R. M., Sayers, A. R. and Woodward, M. J. (2000b) The role of flagella and fimbriae in the colonisation, invasion and persistence of Escherichia coli O78:K80 in the day-old-chick model. Epidemiology and Infection 124, 351-363.

Lactobacillus johnsonii (FI9785) was stored at −80° C. and inocula were cultured in MRS broth for chick experiments. For challenge inocula for chick experiments, broth cultures of S. Enteritidis and E. coli were grown overnight in LB broth at 37° C., aerobically with gentle agitation and broth cultures of C. perfringens were grown in Robertson's Cooked meat broth for 16 h, anaerobically and statically. For selection of bacteria from chick experiments, samples were plated on brilliant green agar (BGA) supplemented with nalidixic acid (15 μgml⁻¹) for S. Enteritidis, Mac-Conkey agar supplemented with nalidixic acid (15 μgml⁻¹) for E. coli O78:K80 or 5% sheep's blood agar for C. perfringens. Incubation was 37° C. for all and anaerobic for C. perfringens. All media were purchased from Oxoid (Basingstoke, UK).

One-Day-Old Chick Model; S. Enteritidis and E. Coli O78:K80 Challenges

Newly hatched chicks were obtained from a specific pathogen-free (SPF) White Leghorn flock specific pathogen free avian supply (SPAFAS). Chicks were housed on wood shavings and fed standard rations and tap water ad libitum. Chicks were observed and weighed regularly.

A total of 120 hatchlings used in the day-old-model study were randomly divided into four groups of 30 birds each. Two groups of 30 birds were dosed orally by gavage as described previously Allen-Vercoe, E., Sayers, R. and Woodward, M. J. (1999) Virulence of Salmonella enterica serovar Enteritidis aflagellate and afimbriate mutants in the day old chick. Epidemiology and Infection 122, 395-402; La Ragione, R. M., Sayers, A. R. and Woodward, M. J. (2000b) The role of flagella and fimbriae in the colonisation, invasion and persistence of Escherichia coli O78:K80 in the day-old-chick model. Epidemiology and Infection 124, 351-363 (within 24 h of hatching with 1×10⁹ CFU Lactobacillus johnsonii (FI9785) suspended in 0.1 ml sterile water. One group of treated and another group of untreated birds were dosed orally by gavage at 48 h of age with 1×10⁵ CFU Salmonella Enteritidis S1400 nal^{r} suspended in 0.1 ml phosphate-buffered saline (PBS). The other two groups, one dosed with L. johnsonii (FI9785) and the other not, were dosed orally by gavage at 48 h of age with 1×10⁵ CFU Escherichia coli O78:K80 nal^{r} suspended in 0.1 ml PBS. At 1, 5, 14 and 36 days postinoculation five birds selected at random from each of the four groups were killed and bacteriological analysis of tissue samples performed.

Twenty-Day-Old Chick Model; C. Perfringens Challenge

A total of 30 newly hatched chicks were obtained from a SPF White Leghorn flock (SPAFAS), housed on wood shavings and fed standard rations and tap water ad libitum. Chicks were observed and weighed regularly.

At 20 days of age, one group of 15 birds was dosed orally by gavage as described previously (La Ragione et al. 2000b, 2001) with 1×10⁹ CFU Lactobacillus johnsonii (FI9785) suspended in 0.1 ml sterile water. This and a control group of 15 birds that had not received L. johnsonii (FI9785) were dosed by gavage 24 h later with 1×10⁵ CFU Clostridium perfringens suspended in 0.1 ml PBS. At 1, 7 and 36 days postinoculation five birds selected at random from each group were killed and bacteriological analysis of tissue samples performed.

Enumeration of S. Enteritidis, E. Coli and C. Perfringens in Tissues

Animals were killed by cervical dislocation, and liver, spleen, duodenum, jejunum, ileum, colon and caeca were removed aseptically from each bird and placed separately in a sterile 1 oz MacCartney glass bottle. Livers and spleens were weighed and each organ was homogenized in a sufficient volume of PBS to give a 1 in 10 dilution factor. The viable count in homogenates of S. Enteritidis was determined by plating dilutions made in PBS (0.1 M, PH 7.2) on BGA supplemented with nalidixic acid (15 μgml⁻¹). The viable count in homogenates of E. coli was determined by plating dilutions made in PBS (0.1 M, pH 7.2) on MacConkey plates supplemented with nalidixic acid (15 μgml⁻¹). The limit of detection was 200 CFU. For both S. Enteritidis and E. coli, 1 ml of residual homogenate was enriched by addition to 20 ml Luriabertani-glucose (LB-G) broth, incubated for 24 h at 37° C. and subcultured on BGA or MacConkey supplemented with nalidixic acid, as appropriate. The viable count of C. perfringens in homogenates was determined by plating dilutions made in PBS (0.1 M, pH 7.2) on blood agar followed by incubation anaerobically, at 37° C. for 16 h.

Semi-Quantitative Enumeration of S. Enteritidis, E. Coli O78:K80 and C. Perfringens by Cloacal Swabbing

The semi-quantitative methods of Smith and Tucker (Smith, H. W. and Tucker, J. F. (1975) The effect of antibiotic therapy on the faecal excretion of Salmonella typhimurium by experimentally infected chickens. Journal of Hygiene (Cambs.), 75, 275-292.; Smith, H. W. and Tucker, J. F. (1980) The virulence of Salmonella strains for chickens: their excretion by infected chickens. Journal of Hygiene (Cambs.), 84, 479-488) were used. Cloacal swabs were taken at weekly intervals from 24 h after challenge. Swabs were taken from 10 birds selected at random at each time point from the S. Enteritidis and E. coli experiments whereas swabs were taken from all remaining birds at each time point from the C. perfringens experiment. To detect and enumerate S. Enteritidis, swabs were spread directly on to BGA plates supplemented with nalidixic acid (15 μgml⁻¹) and the plates were incubated overnight, aerobically at 37° C. To detect and enumerate E. coli O78:K80, swabs were spread directly on to MacConkey plates supplemented with nalidixic acid (15 μgml⁻¹) and the plates were incubated overnight, aerobically at 37° C. To detect and enumerate C. perfringens swabs were spread onto blood agar and the plates were incubated overnight, anaerobically at 37° C. and colony morphology was used to differentiate bacterial types. Results were expressed as heavy (confluent growth), medium (>200 CFU direct plating), or light (<200 CFU direct plating). Single colonies were confirmed as C. perfringens type A by multiplex PCR Yoo, H. S., Lee, S. U., Park, K. Y. and Park, Y. H. (1997) Molecular typing and epidemiological survey of prevalence of Clostridium perfringens types by multiplex PCR. Journal of Clinical Microbiology 35, 228-232.

### Statistical Analysis

For statistical analysis of colonization of S. Enteritidis nal^{r}, E. coli nal^{r} and C. perfringens in birds either dosed previously with L. johnsonii (FI9785) or un-dosed, the number of chicks colonized was assumed to follow binomial distribution. The differences were compared over time for liver, spleen, duodenum, jejunum, ileum, colon and caecum using a nonparametric Mann-Whitney test. The mean and S.D. were transformed to their logarithm to base 10, after adding one to prevent zero counts becoming minus infinity. For swabbing data, a mean score was ascribed (high=4, medium=3, low=2, positive by enrichment=1 and negative=0) for each bird to allow use of analyses of variance techniques. Comparisons were made between levels of shedding separately at each time point and for each bacterium. The probabilities were calculated using the StatXact software program (CYTEL software corp., MA, USA).

### Results

### Growth Inhibition Study

Simple in vitro experiments were performed to test whether L. johnsonii (FI9785) inhibited the growth of S. Enteritidis, E. coli O78:K80 or C. perfringens. To do this, spent MRS medium from a L. johnsonii (FI9785) culture was filter sterilized and added to Robertson's Cooked Meat Broth (RCMB) prior to inoculation with C. perfringens and to nutrient broth prior to inoculation with E. coli or Salmonella, respectively. RCMB and NB without spent MRS were also inoculated as appropriate as positive controls. For each pair of media, the numbers of Clostridia, Salmonella or E. coli recovered from the respective tests after anaerobic or aerobic overnight incubation were similar and not statistically different (data not shown).

TABLE 1
Colonization of 2-day-old birds by Salmonella enteritidis
with and without Lactobacilli predose

| | | | Posi- | Mean | | |
|---|---|---|---|---|---|---|
| Day | | Tissue | tive | counts | | P- |
| P.I. | Treatment | Type | tissues | log10 | S.D. | values |
| 1 | Se alone | Liver | 5/5 | 1.041 | 0.000 | |
| 1 | Se + FI9785 | | 5/5 | 1.041 | 0.000 | 1.000 |
| 5 | Se alone | | 5/5 | 4.341 | 0.478 | |
| 5 | Se + FI9785 | | 5/5 | 3.708 | 0.187 | 0.024 |
| 14 | Se alone | | 4/5 | 0.833 | 0.466 | |
| 14 | Se + FI9785 | | 5/5 | 1.041 | 0.000 | 1.000 |
| 36 | Se alone | | 0/5 | 0.000 | 0.000 | |
| 36 | Se + FI9785 | | 0/5 | 0.000 | 0.000 | 1.000 |
| 1 | Se alone | Spleen | 0/5 | 0.000 | 0.000 | |
| 1 | Se + FI9785 | | 0/5 | 0.000 | 0.000 | 1.000 |
| 5 | Se alone | | 5/5 | 3.154 | 0.283 | |
| 5 | Se + FI9785 | | 5/5 | 3.166 | 0.250 | 1.000 |
| 14 | Se alone | | 5/5 | 1.041 | 0.000 | |
| 14 | Se + FI9785 | | 5/5 | 1.041 | 0.000 | 1.000 |
| 36 | Se alone | | 0/5 | 0.000 | 0.000 | |
| 36 | Se + FI9785 | | 0/5 | 0.000 | 0.000 | 1.000 |
| 1 | Se alone | Duodenum | 5/5 | 1.041 | 0.000 | |
| 1 | Se + FI9785 | | 5/5 | 1.041 | 0.000 | 1.000 |
| 5 | Se alone | | 5/5 | 3.146 | 0.595 | |
| 5 | Se + FI9785 | | 5/5 | 1.456 | 0.927 | 0.024 |
| 14 | Se alone | | 5/5 | 1.041 | 0.000 | |
| 14 | Se + FI9785 | | 2/5 | 0.417 | 0.570 | 0.167 |
| 36 | Se alone | | 0/5 | 0.000 | 0.000 | |
| 36 | Se + FI9785 | | 0/5 | 0.417 | 0.570 | 0.444 |
| 1 | Se alone | Jejunum | 5/5 | 1.041 | 0.000 | |
| 1 | Se + FI9785 | | 5/5 | 1.041 | 0.000 | 1.000 |
| 5 | Se alone | | 5/5 | 1.971 | 1.281 | |
| 5 | Se + FI9785 | | 5/5 | 2.148 | 1.090 | 1.000 |
| 14 | Se alone | | 1/5 | 0.208 | 0.466 | |
| 14 | Se + FI9785 | | 0/5 | 0.000 | 0.000 | 1.000 |
| 36 | Se alone | | 0/5 | 0.000 | 0.000 | |
| 36 | Se + FI9785 | | 0/5 | 0.000 | 0.000 | 1.000 |
| 1 | Se alone | Ileum | 5/5 | 1.041 | 0.000 | |
| 1 | Se + FI9785 | | 5/5 | 1.041 | 0.0.00 | 1.000 |
| 5 | Se alone | | 5/5 | 4.527 | 1.440 | |
| 5 | Se + FI9785 | | 5/5 | 3.774 | 1.030 | 0.452 |
| 14 | Se alone | | 5/5 | 2.948 | 1.088 | |
| 14 | Se + FI9785 | | 3/5 | 0.417 | 0.570 | 0.024 |
| 36 | Se alone | | 4/5 | 2.956 | 2.142 | |
| 36 | Se + FI9785 | | 5/5 | 5.046 | 1.408 | 0.095 |
| 1 | Se alone | Colon | 5/5 | 5.761 | 0.783 | |
| 1 | Se + FI9785 | | 5/5 | 6.655 | 0.179 | 0.135 |
| 5 | Se alone | | 5/5 | 6.962 | 0.089 | |
| 5 | Se + FI9785 | | 5/5 | 6.748 | 0.061 | 0.016 |
| 14 | Se alone | | 5/5 | 6.725 | 0.226 | |
| 14 | Se + FI9785 | | 5/5 | 7.260 | 0.596 | 0.151 |
| 36 | Se alone | | 5/5 | 6.762 | 0.182 | |
| 36 | Se + FI9785 | | 5/5 | 7.045 | 0.074 | 0.008 |
| 1 | Se alone | Caeca | 5/5 | 7.825 | 0.078 | |
| 1 | Se + FI9785 | | 5/5 | 7.239 | 0.458 | 0.143 |
| 5 | Se alone | | 5/5 | 8.732 | 0.030 | |
| 5 | Se + FI9785 | | 5/5 | 8.561 | 0.268 | 0.595 |
| 14 | Se alone | | 5/5 | 8.974 | 0.149 | |
| 14 | Se + FI9785 | | 5/5 | 9.049 | 0.038 | 0.333 |
| 36 | Se alone | | 5/5 | 7.381 | 0.515 | |
| 36 | Se + FI9785 | | 5/5 | 8.016 | 0.257 | 0.079 |

Se = Salmonella enteritidis

Effect of Predosing Birds with L. Johnsonii (FI9785) on the Colonization and Persistence of S. Enteritidis in the 1-Day-Old Chick Model

The numbers of S. Enteritidis nal^{r} recovered from the liver, spleen, duodenum, jejunum, ileum, colon and caeca of chicks 1, 5, 14 and 36 days after challenge are shown in Table 1. The presence of L. johnsonii (FI9785) made no difference upon the invasion, colonization and clearance of the deep tissues (livers and spleens). For all of the GI tissues tested, the recovery of S. Enteritidis was similar irrespective of the presence or absence of L. johnsonii (FI9785). For the small intestine there appeared to be no consistent trend. However, with regard to the large intestine, the numbers of S. Enteritidis recovered at days 14 and 36 were higher with a L. johnsonii (FI9785) predose than without. One of these values was statistically significant (colon day 36; P=0.008) and another approached significance (caeca day 36; P=0.079).

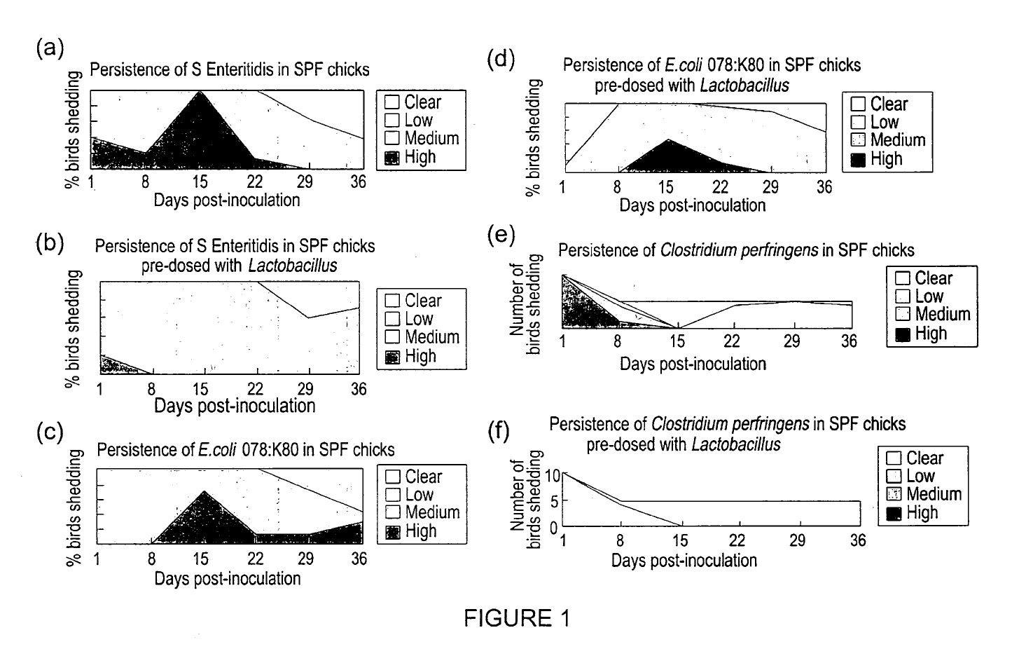

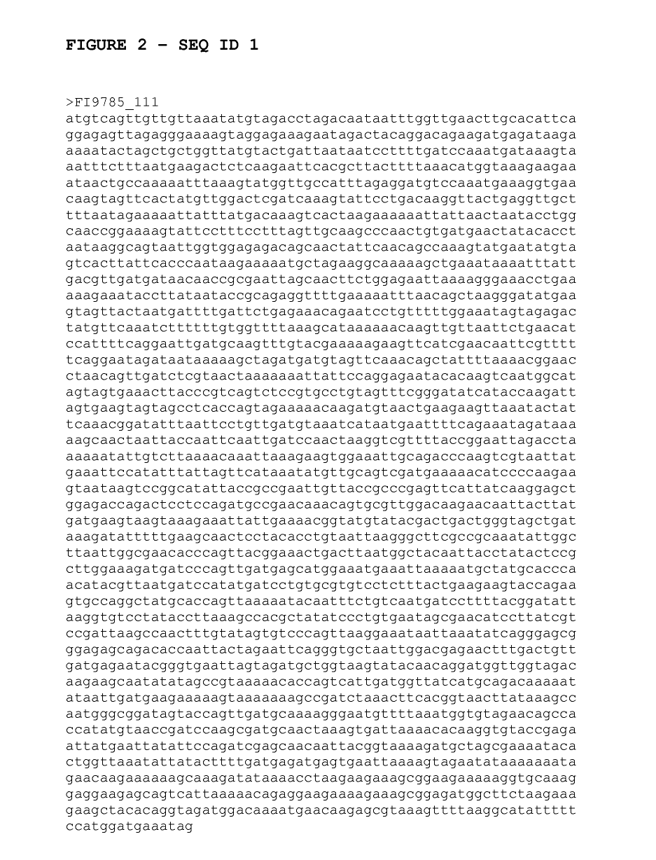

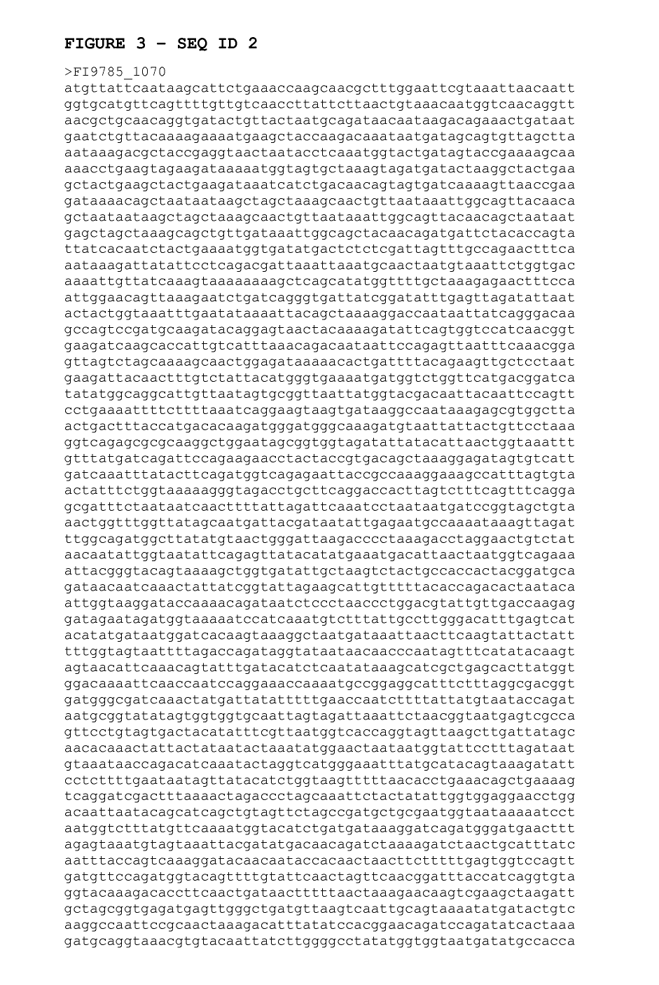

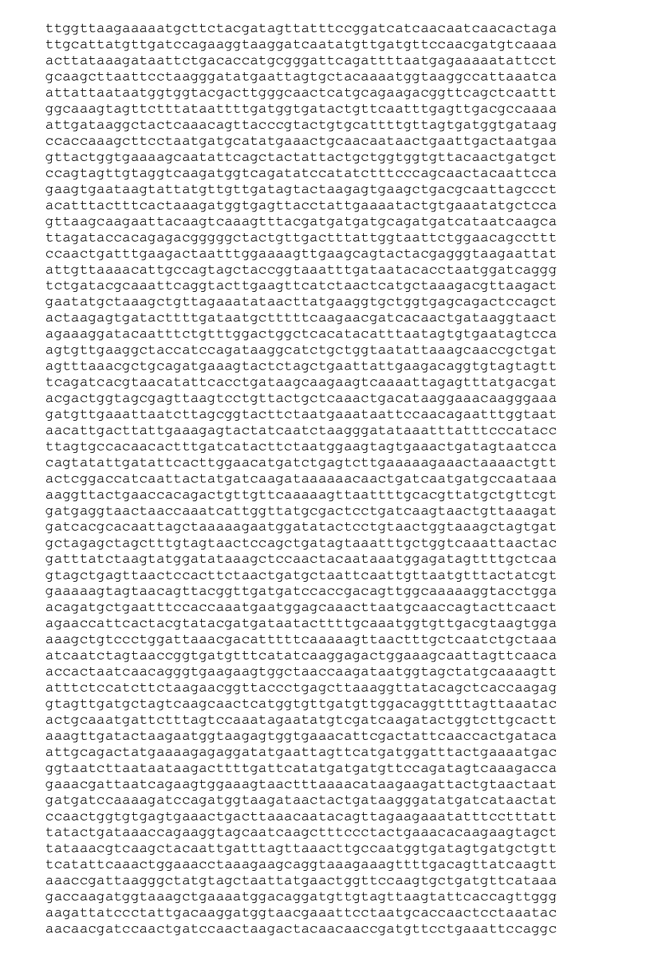

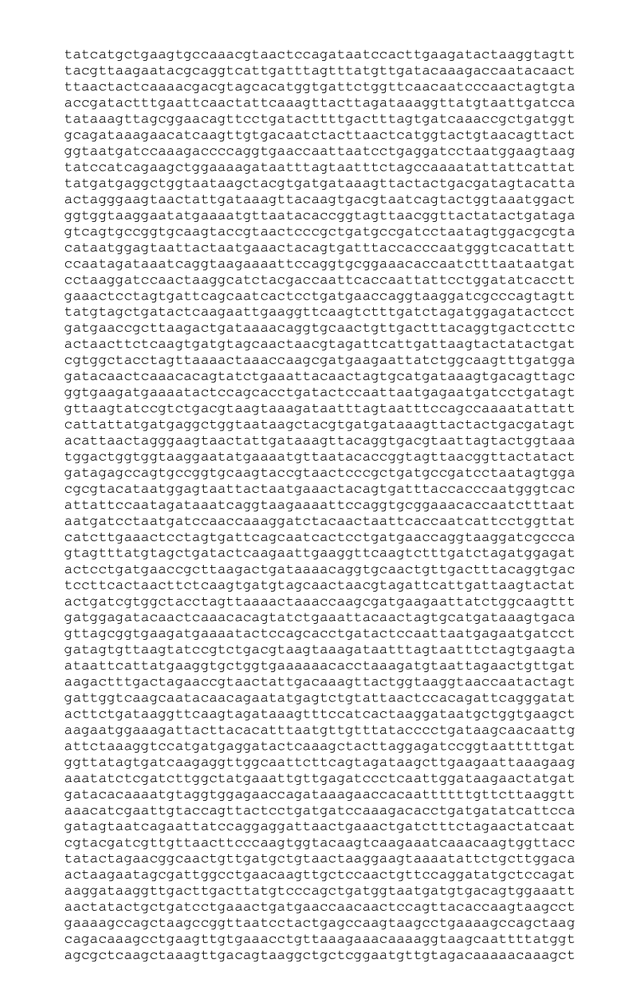

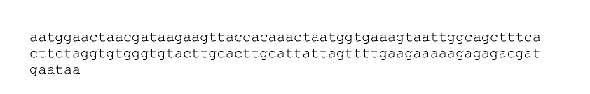

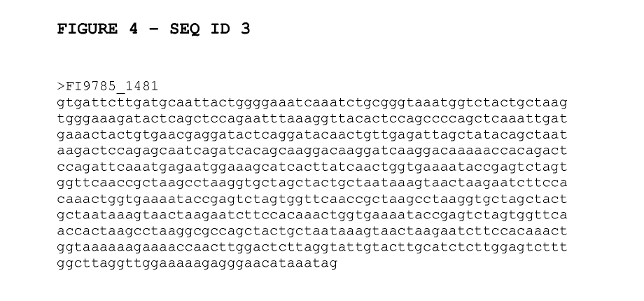

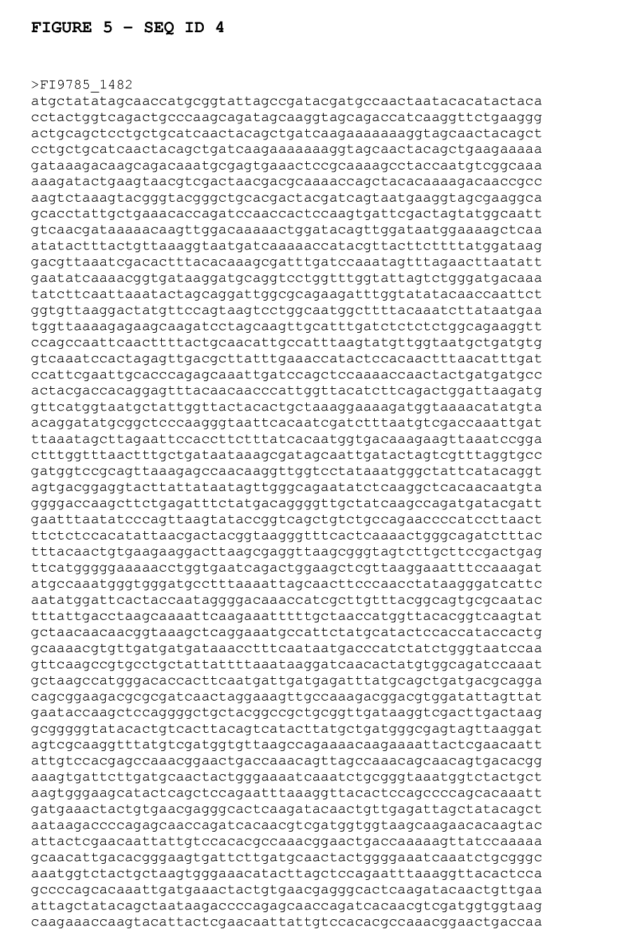

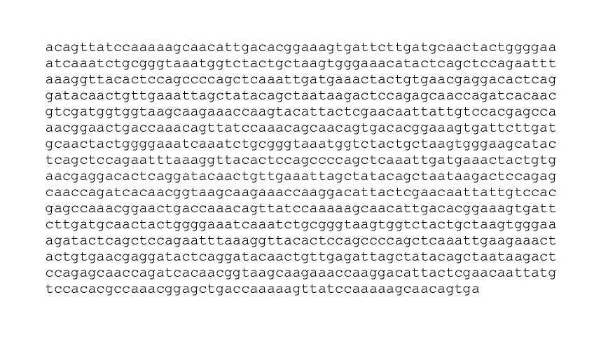

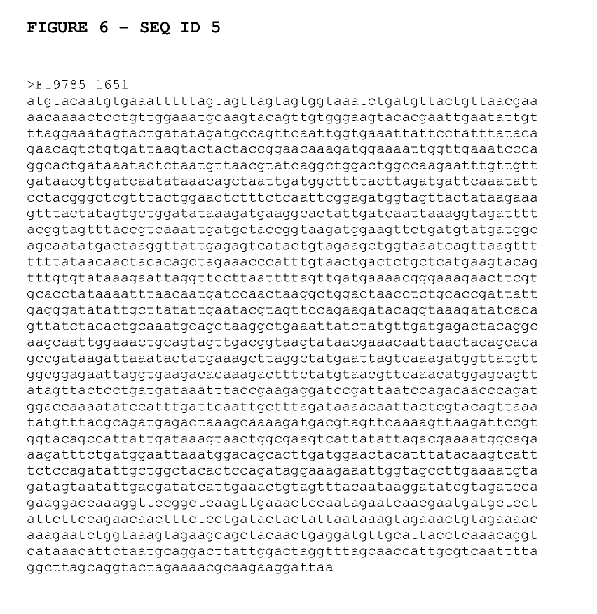

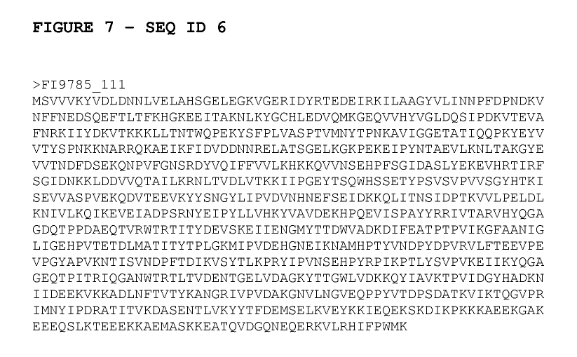

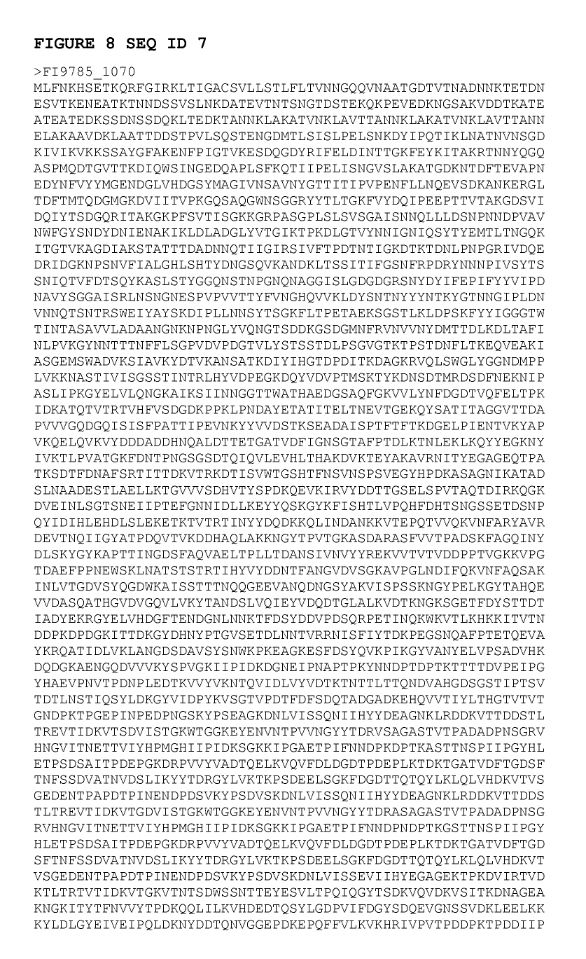

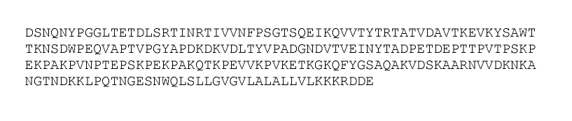

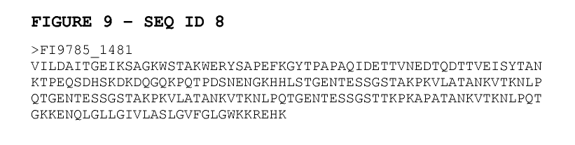

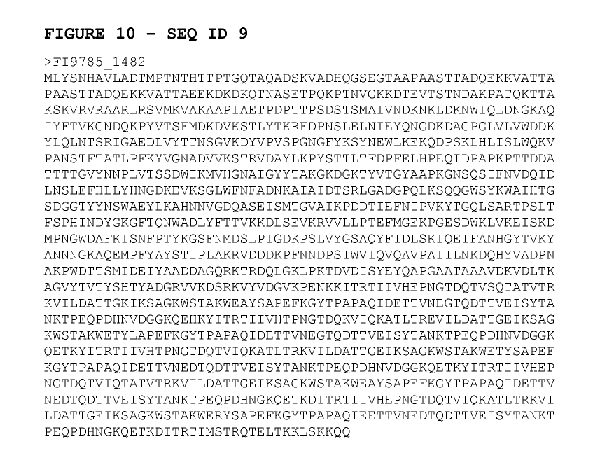

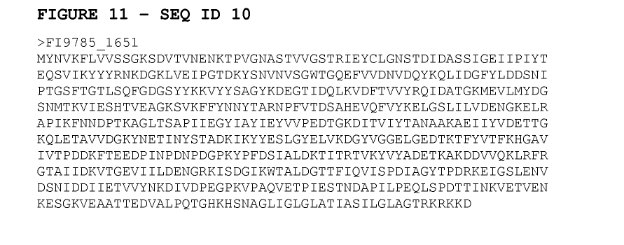

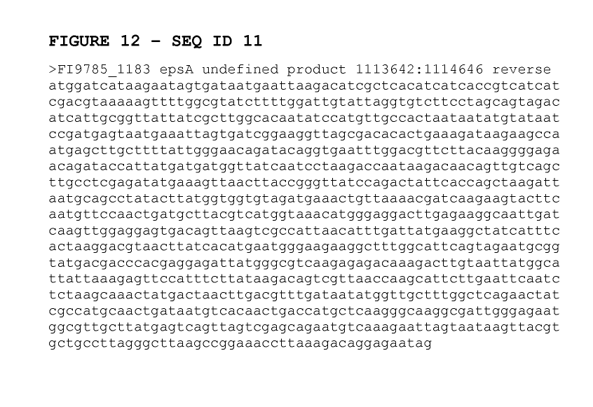

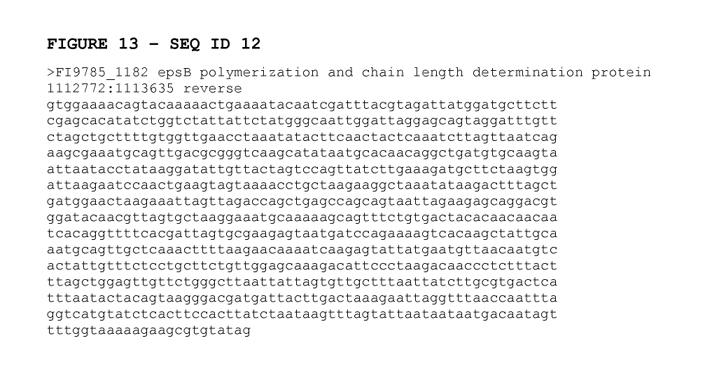

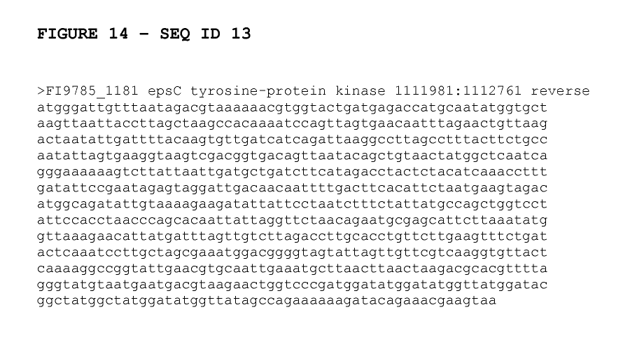

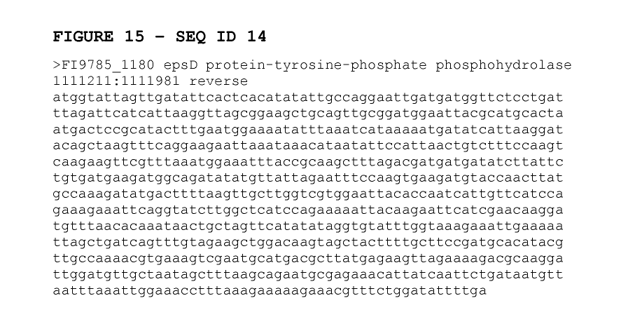

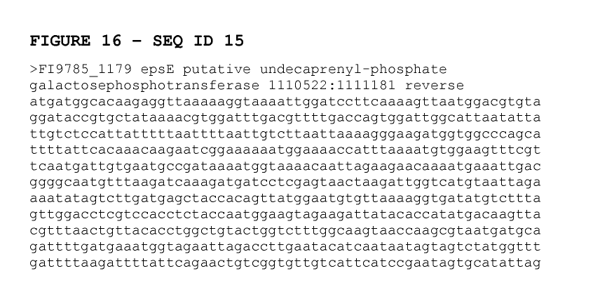

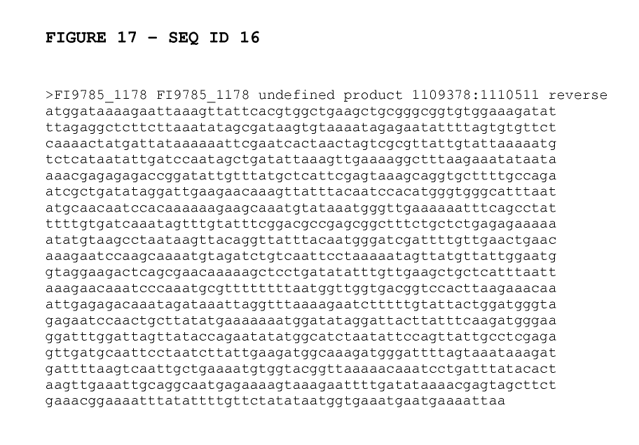

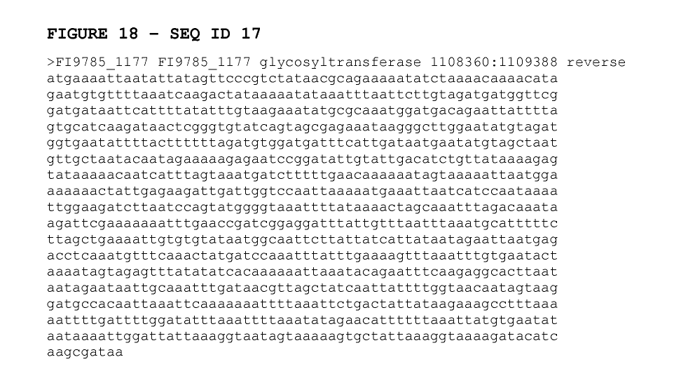

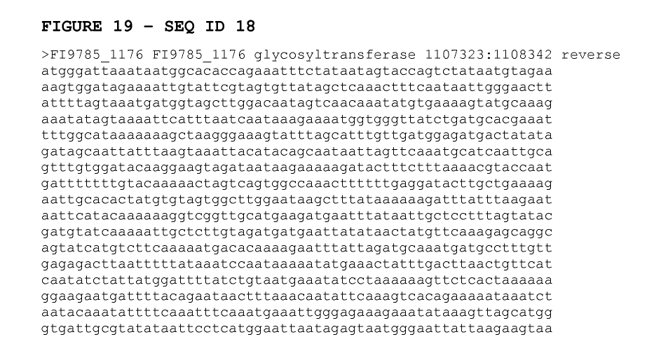

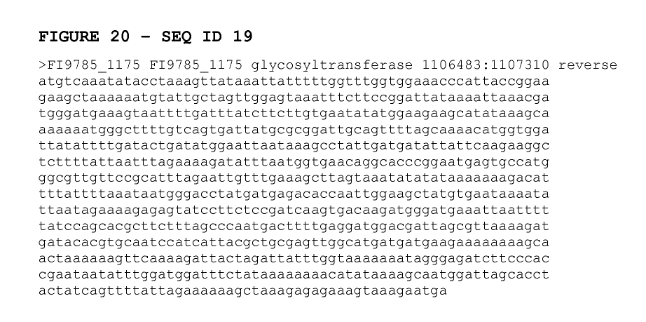

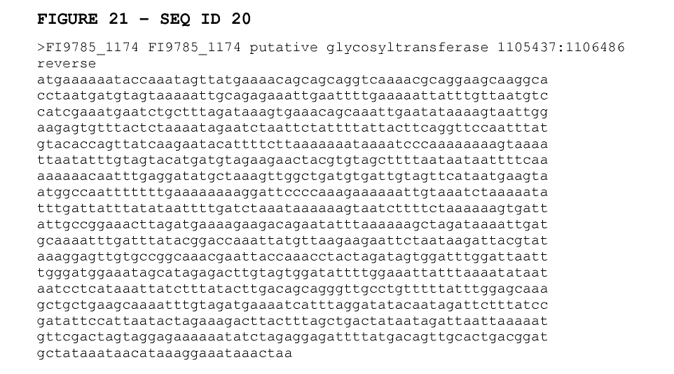

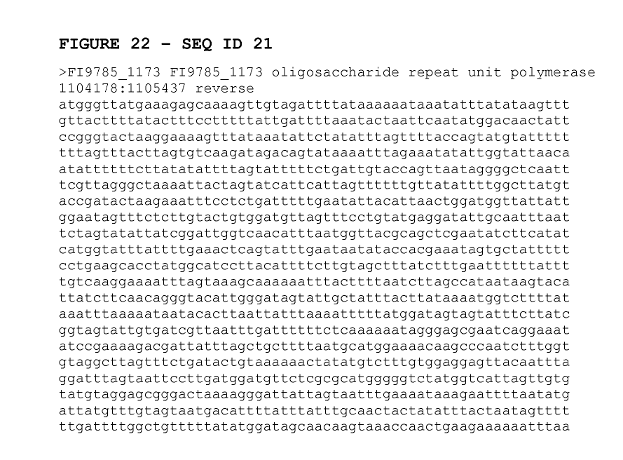

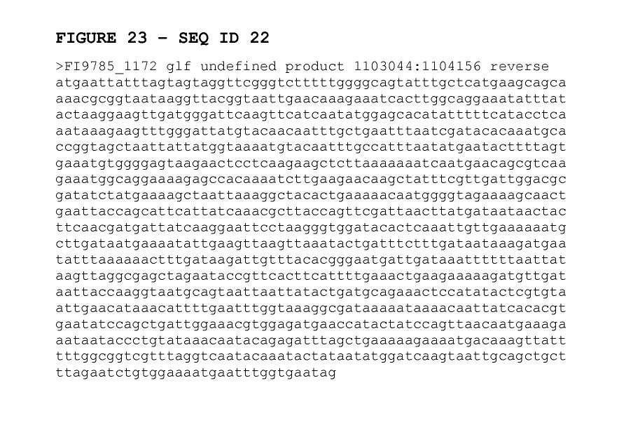

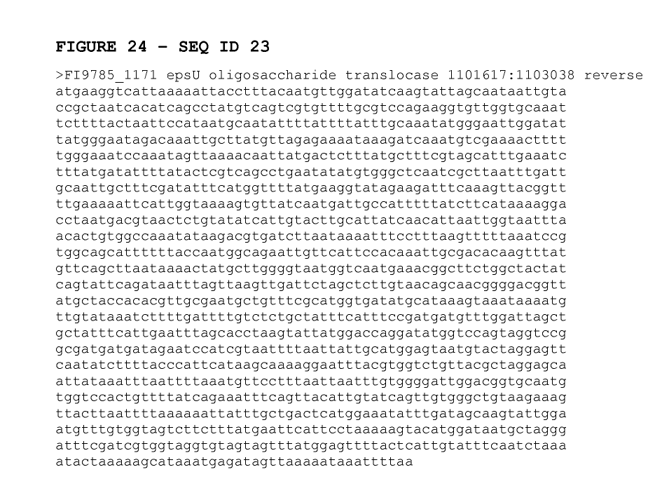

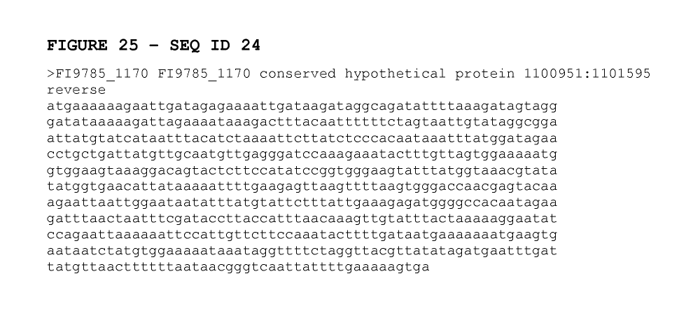

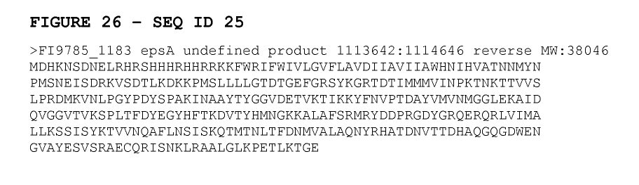

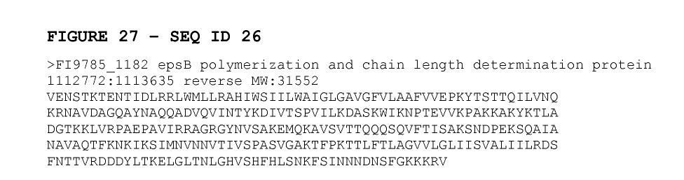

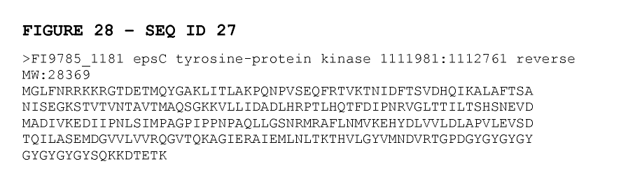

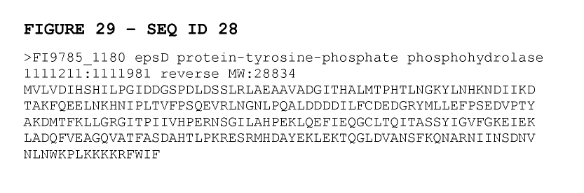

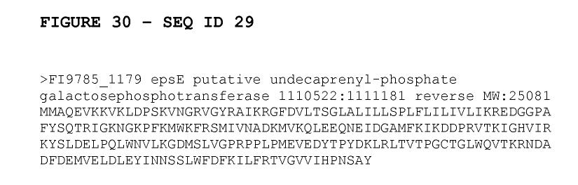

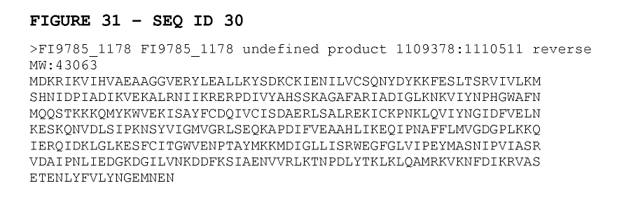

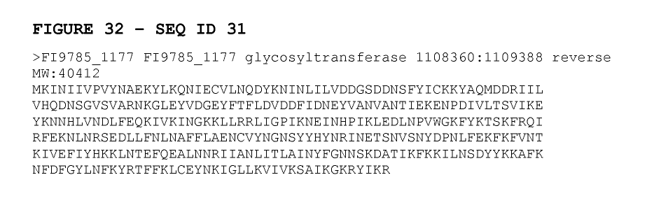

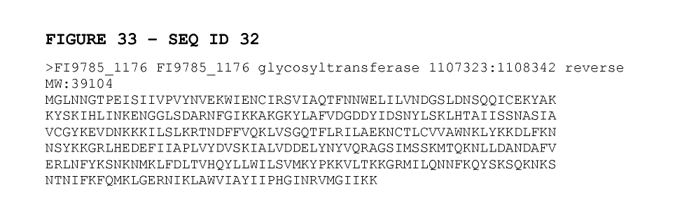

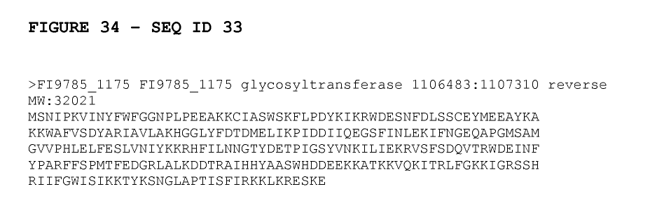

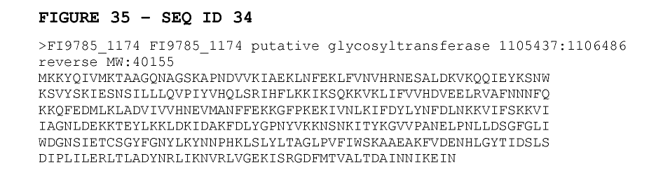

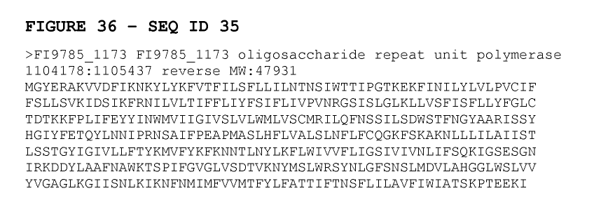

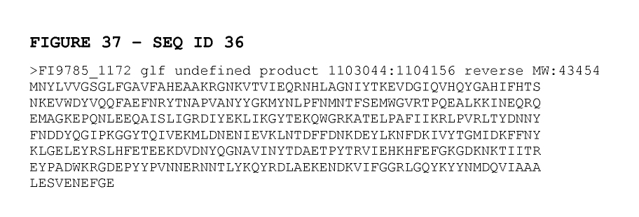

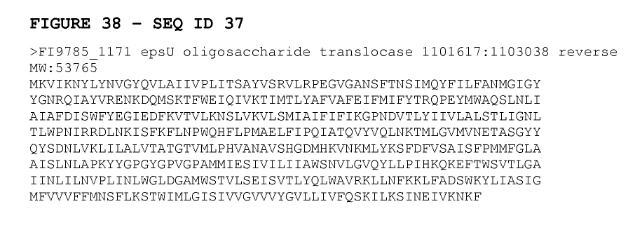

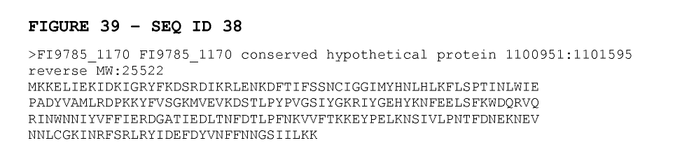

Shedding of S. Enteritidis nal^{r} was monitored by cloacal swabbing weekly and the data are presented graphically in FIG. 1a,b. There was significantly lower shedding from birds predosed with L. johnsonii (FI9785) on day 15 (P<0.001) but not on any other sampling day.

Effect of Predosing Birds with L. Johnsonii (FI9785) on the Colonization and Persistence of E. Coli O78:K80 in the 1-Day-Old Chick Model

The numbers of E. coli O78:K80 nal^{r} recovered from the liver, spleen, duodenum, jejunum, ileum, colon and caeca of chicks 1, 5, 14 and 36 days after challenge are shown in Table 2. Bacteriological analysis of deep tissues (livers and spleens) showed a trend toward lower recovery of E. coli from birds predosed with L. johnsonii (FI9785) but the differences were not statistically significant. With regard to recovery of E. coli from the small intestine, there were significantly fewer organisms from 10 of 12 samples from birds predosed with L. johnsonii (FI9785). However, recovery of E. coli from the caeca and colon from, birds predosed with L. johnsonii (FI9785) yielded significantly fewer (P<0.008) organisms than without the predose but only at 24 h after challenge. There after, the numbers of E. coli in predosed and control birds were similar.

Shedding of E. coli O78:K80 nal^{r} was monitored by cloacal swabbing and the data are shown graphically in FIG. 1c,d. The shedding of E. coli from birds predosed with L. johnsonii (FI9785) was reduced significantly compared with controls 1 day after challenge (P<0.001). Thereafter, a modest trend of lower shedding was noted but was not statistically significant (P<0.275).

Effect of Predosing Birds with L. Johnsonii (FI9785) on the Colonization and Persistence of C. Perfringens in the 20-Day-Old Chick Model

The numbers of C. perfringens recovered from the liver, spleen, duodenum, jejunum, ileum, colon and caeca of chicks 1, 7 and 36 days after challenge are shown in Table 3. Clostridium perfringens was recovered from the livers and spleens of three of five control birds 1 day after challenge but not from any birds predosed with L. johnsonii (FI9785) and thereafter no liver or spleen tissues were positive from either group for the duration of the experiment. These data were not statistically significant. With regard to the GI tract a clear trend was shown. Of the 60 tissues from control and predose groups that were examined over the time course of the experiment, 54 were positive for C. perfringens from control birds whilst 29 were positive from birds predosed with L. johnsonii (FI9785). Additionally, the numbers of C. pefringens recovered were lower from positive birds if predosed with Lactobacillus johnsonii (FI9785). Furthermore, these differences increased over the time course of the experiment. Collectively, seven of the 15 tissue comparisons showed statistically significant differences (P=0.05 or less).

Shedding of C. perfringens was monitored by cloacal swabbing on days 1, 8, 15, 22, 29 and 36 after challenge and the data are shown graphically in FIG. 1e,f. There was significantly lower shedding from birds predosed with Lactobacillus johnsonii (FI9785) on 5 of the 6 days tested with day 15 showing no statistically significant differences (P-values <0.001, =0.040, 1.000, =0.048, =0.008, =0.048).

TABLE 2
Colonization of 2-day-old birds by Escherichia coli
078:K80 with and without Lactobacilli predose

| | | | | Mean | | |
|---|---|---|---|---|---|---|
| Day | | Tissue | Positive | counts | | |
| P.I. | Treatment | type | tissues | log₁₀ | SD | P = values |
| 1 | Ec alone | Liver | 4/5 | 2-011 | 1.487 | |
| 1 | Ec + FI9785 | | 5/5 | 1-041 | 0-000 | 0-325 |
| 5 | Ec alone | | 5/5 | 3-098 | 2-229 | |
| 5 | Ec + FI9785 | | 5/5 | 1-041 | 0-000 | 0-309 |
| 14 | Ec alone | | 1/5 | 0-208 | 0-466 | |
| 14 | Ec + FI9785 | | 0/5 | 0-000 | 0-000 | 1-000 |
| 36 | Ec alone | | 0/5 | 0-000 | 0-000 | |
| 36 | Ec + FI9785 | | 0/5 | 0-000 | 0-000 | 1-000 |
| 1 | Ec alone | Spleen | 5/5 | 2-311 | 1-223 | |
| 1 | Ec + FI9785 | | 3/5 | 0-625 | 0-570 | 0-079 |
| 5 | Ec alone | | 5/5 | 1-041 | 0.000 | |
| 5 | Ec + FI9785 | | 5/5 | 1-041 | 0-000 | 1-000 |
| 14 | Ec alone | | 0/5 | 0-000 | 0-000 | |
| 14 | Ec + FI9785 | | 0/5 | 0-000 | 0-000 | 1-000 |
| 36 | Ec alone | | 0/5 | 0-000 | 0-000 | |
| 36 | Ec + FI9785 | | 0/5 | 0-000 | 0-000 | 1-000 |
| 1 | Ec alone | Duodenum | 5/5 | 2-674 | 1-342 | |
| 1 | Ec + FI9785 | | 5/5 | 1-041 | 0-000 | 0-048 |
| 5 | Ec alone | | 5/5 | 2-825 | 2-310 | |
| 5 | Ec + FI9785 | | 5/5 | 1-041 | 0-000 | 0-167 |
| 14 | Ec alone | | 5/5 | 4-867 | 0-823 | |
| 14 | Ec + FI9785 | | 0/5 | 0-000 | 0-000 | 0-008 |
| 36 | Ec alone | | 0/5 | 0-000 | 0-000 | |
| 36 | Ec + FI9785 | | 5/5 | 4-379 | 2-019 | 0-008 |
| 1 | Ec alone | Jejunum | 5/5 | 5-601 | 1-553 | |
| 1 | Ec + FI9785 | | 5/5 | 1-041 | 0-000 | 0-008 |
| 5 | Ec alone | | 5/5 | 4-234 | 1-110 | |
| 5 | Ec + FI9785 | | 5/5 | 2-475 | 1-633 | 0-008 |
| 14 | Ec alone | | 5/5 | 5-044 | 1-516 | |
| 14 | Ec + FI9785 | | 1/5 | 0-208 | 0-466 | 0-008 |
| 36 | Ec alone | | 5/5 | 2-058 | 1-395 | |
| 36 | Ec + FI9785 | | 5/5 | 5-640 | 0-832 | 0-008 |
| 1 | Ec alone | Ileum | 5/5 | 4-657 | 1-090 | |
| 1 | Ec + FI9785 | | 5/5 | 1-041 | 0-000 | 0-008 |
| 5 | Ec alone | | 5/5 | 4-752 | 0-560 | |
| 5 | Ec + FI9785 | | 5/5 | 3-801 | 0-695 | 0-064 |
| 14 | Ec alone | | 5/5 | 5-879 | 0-874 | |
| 14 | Ec + FI9785 | | 5/5 | 3-025 | 1-705 | 0-008 |
| 36 | Ec alone | | 5/5 | 5-921 | 1-111 | |
| 36 | Ec + FI9785 | | 5/5 | 7-354 | 1-130 | 0-008 |
| 1 | Ec alone | Colon | 5/5 | 6-961 | 0-274 | |
| 1 | Ec + FI9785 | | 5/5 | 5-158 | 0-444 | 0-008 |
| 5 | Ec alone | | 5/5 | 6-495 | 0-683 | |
| 5 | Ec + FI9785 | | 5/5 | 6-743 | 0-317 | 0-841 |
| 14 | Ec alone | | 5/5 | 7-140 | 0-108 | |
| 14 | Ec + FI9785 | | 5/5 | 7-234 | 0-072 | 0-127 |
| 36 | Ec alone | | 5/5 | 7-286 | 0-125 | |
| 36 | Ec + FI9785 | | 5/5 | 7-454 | 0-037 | 0-0324 |
| 1 | Ec alone | Caeca | 5/5 | 9-058 | 0-035 | |
| 1 | Ec + FI9785 | | 5/5 | 8-758 | 0-196 | 0-008 |
| 5 | Ec alone | | 5/5 | 8-514 | 0-426 | |
| 5 | Ec + FI9785 | | 5/5 | 8-054 | 0-532 | 0-175 |
| 14 | Ec alone | | 5/5 | 8-845 | 0-240 | |
| 14 | Ec + FI9785 | | 5/5 | 8-824 | 0-197 | 0-730 |
| 36 | Ec alone | | 5/5 | 8-906 | 0-135 | |
| 36 | Ec + FI9785 | | 5/5 | 8-765 | 0-115 | 0-183 |

Ec, Escherichia coli

TABLE 3
Colonization of 21-day-old birds by Clostridium perfringens
with and without lactobacilli predose

| | | | | Mean | | |
|---|---|---|---|---|---|---|
| Day | | Tissue | Positive | counts | | |
| P.I. | Treatment | type | tissues | log₁₀ | S.D. | P-values |
| 1 | CP alone | Liver | 3/5 | 1-591 | 1-457 | |
| 1 | CP + FI9785 | | 0/5 | 0-000 | 0-000 | 0-167 |
| 7 | CP alone | | 0/5 | 0-000 | 0-000 | |
| 7 | CP + FI9785 | | 0/5 | 0-000 | 0-000 | 1-000 |
| 36 | CP alone | | 0/5 | 0-000 | 0-000 | |
| 36 | CP + FI9785 | | 0/5 | 0-000 | 0-000 | 1-000 |
| 1 | CP alone | Spleen | 3/5 | 1-652 | 1-577 | |
| 1 | CP + FI9785 | | 0/5 | 0-000 | 0-000 | 0-167 |
| 7 | CP alone | | 0/5 | 0-000 | 0-000 | |
| 7 | CP + FI9785 | | 0/5 | 0-000 | 0-000 | 1-000 |
| 36 | CP alone | | 0/0 | 0-000 | 0-000 | |
| 36 | CP + FI9785 | | 0/0 | 0-000 | 0-000 | 1-000 |
| 1 | CP alone | Duodenum | 5/5 | 2-656 | 0-250 | |
| 1 | CP + FI9785 | | 1/5 | 0-600 | 1-342 | 0-143 |
| 7 | CP alone | | 4/5 | 2-071 | 1-211 | |
| 7 | CP + FI9785 | | 0/5 | 0-000 | 0-000 | 0-048 |
| 36 | CP alone | | 5/5 | 3-172 | 0-640 | |
| 36 | CP + FI9785 | | 1/5 | 0-629 | 1-407 | 0-032 |
| 1 | CP alone | Jejunum | 5/5 | 3-180 | 0-282 | |
| 1 | CP + FI9785 | | 1/5 | 0-600 | 1-342 | 0-024 |
| 7 | CP alone | | 2/5 | 0-921 | 1-262 | |
| 7 | CP + FI9785 | | 0/5 | 0-000 | 0-000 | 0-444 |
| 36 | CP alone | | 5/5 | 2-796 | 0-242 | |
| 36 | CP + FI9785 | | 0/5 | 0-000 | 0-000 | 0-008 |
| 1 | CP alone | Ileum | 5/5 | 3-702 | 0-898 | |
| 1 | CP + FI9785 | | 5/5 | 3-103 | 0-609 | 2-46 |
| 7 | CP alone | | 5/5 | 3-673 | 0-695 | |
| 7 | CP + FI9785 | | 4/5 | 1-061 | 1-524 | 0-032 |
| 36 | CP alone | | 5/5 | 4-303 | 0-047 | |
| 36 | CP + FI9785 | | 1/5 | 0-623 | 1-393 | 0-008 |
| 1 | CP alone | Colon | 5/5 | 3-085 | 0-180 | |
| 1 | CP + FI9785 | | 3/5 | 1-862 | 1-720 | 0-460 |
| 7 | CP alone | | 5/5 | 4-075 | 0-304 | |
| 7 | CP + FI9785 | | 4/5 | 2-523 | 1-539 | 0-032 |
| 36 | CP alone | | 5/5 | 3-811 | 0-591 | |
| 36 | CP + FI9785 | | 0/5 | 0-000 | 0-000 | 0-008 |
| 1 | CP alone | Caeca | 3/5 | 2-058 | 1-967 | |
| 1 | CP + FI9785 | | 0/5 | 0-000 | 0-000 | 0-167 |
| 7 | CP alone | | 5/5 | 4-156 | 0-128 | |
| 7 | CP + FI9785 | | 5/5 | 3-560 | 0-077 | 0-008 |
| 36 | CP alone | | 4/5 | 2-633 | 1-524 | |
| 36 | CP + FI9785 | | 4/5 | 2-493 | 1-711 | 0-810 |

CP, Clostridium perfringens.

Persistence of L. Johnsonii (FI9785) in 1-Day and 20-Day-Old Chicks

Swabs were also plated onto media selecting for L. johnsonii on MRS media in order to test persistence in the chicken in both the 1- and 20-day-old models. Lactobacilli were recovered from the chickens for the duration of the experiment, albeit in declining numbers over the time course of the experiment, from both experimental age groups.

Previous studies have demonstrated that Lactobacilli given to poultry to control carriage of bacterial pathogens has met with varying degrees of success depending on the strains as CE agent, the pathogens tested and the method of assessment used. For example, Soerjadi, A. S., Stehman, S. M., Snoeyenbos, G. H., Weinack, O. M. and Smyser, C. F. (1981) The influence of lactobacilli on the CE of paratyphoid salmonellae in chickens. Avian Diseases 25, 1027-1033 showed that native Lactobacilli isolated from chickens colonized the crop and caeca of poultry well and reduced the number of Salmonellae colonizing the crop mucosa by a factor of 1 to 2 log₁₀ whereas protective effects were not noted elsewhere. Similar observations have been made by others also.

Watkins, B. A. and Miller, B. F. (1983) Competitive gut exclusion of avian pathogens by Lactobacillus acidophilus in gnotobiotic chicks, Poultry Science 62, 1772-1779 showed that L. acidophilus colonized the crop and caeca of poultry well but resulted in a significant reduction of Staphylococci and S. Typhimurium only in the crop. Likewise, Rada, V. and Rychly, I. (1995) The effect of Lactobacillus salivarius administration on coliform bacteria and enterococci in the crop and cecum of broiler chickens. Veterinary Medicine (Praha) 40, 311-315 showed that L. salivarius 51R reduced enterococci and coliforms in the crop but not the caecum.

Thus, the data disclosed here focussed upon the potential protective effect of L. johnsonii (FI9785) against a number of pathogenic bacteria as assessed by enumerating bacteria throughout the GI tract, without concentrating on the crop, and in faeces by taking cloacal swabs. We found that a single oral dose of 1×10⁹ CFU of L. johnsonii (FI9785) given to 20-day-old chicks reduced the extent of colonization and persistence of C. perfringens in this model. However, the same agent failed to induce any significant and lasting protective effect in the 1-day-old chick model challenged with either S. Enteritidis or E. coli.

Colonization of the avian GI tract with C. perfringens is well recognized. It is also known that necrotic enteritis may be induced in colonized birds by various environmental stresses or dietary changes with manifestation of clinical signs most commonly in 14-20-day-old chicks Ficken, M. D. and Wages, D. P. (1997) Necrotic enteritis. In Diseases of Poultry, 10th edn ed. Calnek, B. W., Barnes, H. J., Beard, C. W., McDougald, L. R. and Saif, Y. M. pp. 261-264. Ames, Iowa: Iowa State University Press; Van der Sluis, W. (2000a) Clostridial enteritis—a syndrome emerging world-wide. World Poultry 16, 56-57 and Van der Sluis, W. (2000b) Clostridial enteritis is an often underestimated problem. World Poultry 16, 42-43.

The model used herein was developed to imitate late colonization but without induction of disease. The presence of L. johnsonii (FI9785) was associated with a significant reduction in colonization of the entire GI tract. Furthermore, the numbers of C. perfringens in the predosed birds did not approach those attained in the controls at any site and the highest numbers attained in the predosed strains was the ileum. It is possible that this site was a primary colonization site for the strain of C. perfringens used or that the L. johnsonii (FI9785) exerted a reduced effect here.

The reduced level of GI colonization equated well with the reduction of shedding of C. perfringens as assessed by cloacal swabbing.

An established 1-day-old chick model to assess the effects of L. johnsonii (FI9785) upon colonization and persistence of S. Enteritidis and E. coli showed that the protective effects were negligible against S. Enteritidis, findings in common with Weinack, O. M., Snoeyenbos, G. H. and Soerjadi-Liem, A. S. (1985) Further studies on CE of Salmonella typhimurium by lactobacilli in chickens. Avian Diseases 29, 1273-1276 who used challenge models to assess single or multiple strain inoculum comprising up to six different Lactobacilli given to 1-day-old chicks. They showed that the treatments investigated had no effect on the outcome of challenge with pathogens such as S. Typhimurium, as assessed by the enumeration of Salmonella recovered in faeces and cloacal swabs. However, Pascual, M., Hugas, M., Badiola, J. I., Montford, J. M. and Garriga, M. (1999) Lactobacillus salivarius CTC2197 prevents Salmonella enteritidis colonisation in chickens. Applied and Environmental Microbiology 65, 4981-4986 showed that L. salivarius CTC2197 excluded S. Enteritidis completely by 21 days after challenge in poultry models similar to those described here. Thus, here is evidence that the model we used was appropriate but that the specific strain used, L. johnsonii (FI9785), had no effect against S. Enteritidis.

With regard to the effect of L. johnsonii (FI9785) against E. coli, there was evidence of a delay of invasion and colonization of the deep organs and a delay of the colonization of all sites of the GI tract. Furthermore, whereas the large intestine did become well colonized beyond day 1 after challenge, the small intestine showed statistically significant reduction of colonization thereafter. These data suggest that L. johnsonii (FI9785) did exert effects against E. coli in the small intestine but that the overall effects did not reduce colonization of the caecum and faecal output.

There was no evidence from the limited in vitro studies performed to suggest inhibition of growth of any of the pathogenic strains used in this study. Therefore, the effects of L. johnsonii (FI9785) upon C. perfringens and E. coli were most probably an in vivo-induced phenomenon.

Although not specifically evaluated, there was no evidence to suggest that L. johnsonii (FI9785) had any adverse effect on the birds and it may be deduced that the beneficial effects observed were as a result of competitive exclusion. Schneitz, C. and Mead, G. (2000) Competitive exclusion. In Salmonella in Domestic Animals ed. Wray, C. and Wray, A. pp. 301-322 suggested that CE agents exert their effect by one or more of four general principle actions, namely the creation of a restrictive physiological environment, competition for bacterial receptor sites, depletion of essential substrates and/or elaboration of antibiotic like substances. The latter may be discounted in this case but whatever the mode of action the effect of L. johnsonii (FI9785) upon C. perfringens and E. coli was immediate and long lasting. The effect against C. perfringens was sustained throughout all sites of the GI tract whereas against E. coli the effect was limited to the small intestine only. Thus it seems highly unlikely that the mode of the effect was the same against both organisms and it is possible that L. johnsonii (FI9785) may have modified the balance of the resident or developing microflora in the bird such that it was that modification that was protective. An additional consideration is the number of doses of L. johnsonii (FI9785). A single bolus given 24 hour prior to challenge gave some protective effect against challenge strains and it may be possible that multiple doses may enhance the effect. Effects against both Gram-positive and Gram-negative bacteria have been reported previously Watkins, B. A. and Miller, B. F. (1983) Competitive gut exclusion of avian pathogens by Lactobacillus acidophilus in gnotobiotic chicks, Poultry Science 62, 1772-1779.

In addition, because single micro-organism based CE agents have been considered to be limited in effect Stavric, S. (1992) Defined cultures and prospects for probiotics. International Journal of Food Microbiology 15, 173-180), it may be possible to extend the range of organisms against which treatments may be developed by the use of combined CE agent and antibodies. For example, the studies of Promsopone, B., Morishita, T. Y., Aye, P. P., Cobb, C. W., Veldkamp, A. and Clifford, J. R. (1998) Evaluation of an avian-specific probiotic and Salmonella typhimurium-specific antibodies on the colonization of Salmonella typhimurium in broilers. Journal of Food Protection 61, 176-180 and Tellez, G., Petrone, V. M., Escorcia, M., Morishita, T. Y., Cobb, C. W., Villasenor, L. and Promsopone, B. (2001) Evaluation of avianspecificprobiotic and Salmonella enteritidis-, Salmonella typhimurium-, and Salmonella heidelberg-specific antibodies on cecalcolonization and organ invasion of Salmonella enteritidis in broilers. Journal of Food Protection 64, 287-291 have combined L. acidophilus, Streptococcus faecium with S. Typhimurium, S. Enteritidis and S. Heidelberg specific antibodies for use in poultry.

Of importance, we have shown that L. johnsonii (FI9785) given as an oral bolus to poultry interferes with the colonization and persistence of two bacterial pathogens. In combination with other agents, the range of activity against other pathogens may be extended. For example, Fukata, T., Tsutsui, H., Baba, E. and Arakawa, A. (1991) Population of Salmonella serovar typhimurium in the caecum of gnotobiotic chickens with Escherichia coli and intestinal bacteria. Journal of Veterinary and Medical Science 53, 229-232 showed that a multivalent agent comprising L. acidophilus, Bifidobacterium thermophiles, Bacteroides vulgates and C. perfringens suppressed the colonization of poultry by S. Typhimurium. However, in this case it is questionable whether an agent containing C. perfringens would be acceptable but it does raise the issue of one pathogen interfering with another in vivo.

We have shown previously that Bacillus subtilis interferes with C. perfringens and S. Enteritidis La Ragione, R. M. and Woodward, M. J. (2003) CE by Bacillus subtilis spores of Salmonella enterica serotype Enteritidis and Clostridium perfringens in young chickens. Veterinary Microbiology, 94, 245-256 which leads us to conclude that an agent that combined L. johnsonii FI9785 and B. subtilis would have utility as a CE agent.

Additional studies have shown that L. johnsonii (FI9785) has use in controlling other pathogens such as Campylobacter jejuni Sorokulova, I. B., Kirik, D. L. and Pinchuk, I. V. (1997) Probiotics against Campylobacter pathogens. Journal of Traveller's Medicine 4, 167-170.

Accordingly it appears that this strain either alone or in combination with other CE agents may have an especially protective effect against a wide range of pathogens including C. perfringens, E. coli, and Campylobacter jejuni in poultry.

### Example 2

### In Vitro Study Showing Adhesion of L. Johnsonii to Human Tissue in Culture

Five putative probiotic Lactobacilli strains obtained from caeca of poultry GI tract were designated FI9785, FI9786, FI9791, FI9794, and S89 isolates and were tested for their ability to adhere to tissue cell lines. Stocks of all bacterial strains were maintained in 40% glycerol at −70° C. All Lactobacilli cultures were grown in MRS medium.

### Colonic Cell Culture

The HEp-2, human colon adenocarcinoma, cell line was used for all adhesion assays. The cells were cultured in 5A McCoy's tissue culture media (Sigma) containing 10% heat inactivated foetal calf serum (Sigma), 2% Pen-Strep (Invitrogen) and 1% GlutaMax™ media (Invitrogen). The cells were grown at 37° C. in 5% CO₂ and the tissue culture media was replaced every two days. For the adhesion assays, monolayers of HEp-2 cells were grown in 24 well plates until 85-100% confluent. The wells were initially seeded at 5×10⁵ cells/ml and reached confluence within 72 h.

### Adhesion Assays

The adhesion of each of the seven Lactobacilli strains to the Hep-2 monolayers (within the 24 well plates) was measured. Each well containing tissue monolayer was washed three times with antibiotic-free tissue culture (TC) medium. The monolayers were then inoculated with 3×10⁸ Lactobacilli cells; administered as a bacterial suspension in 1 ml of TC media. The assay was conducted in triplicate for each of the bacterial strains. The 24 well plates were incubated at 37° C. in 5% CO₂ for 3.5 h. The wells were then washed three times, with antibiotic free TC media, to remove non-adherent bacteria. 1 ml of Triton x100 (1% v/v, Sigma) and a magnetic flea were then added to each well. The contents of the wells were subsequently agitated (by the fleas) at medium/low speed for 10 minutes to dislodge the monolayer. 20 μl of the resulting supernatant was pipetted from each well for preparation of serial dilutions. Each dilution was then plated on MRS plates and incubated overnight at 37° C. to establish relative number of adherent cells.

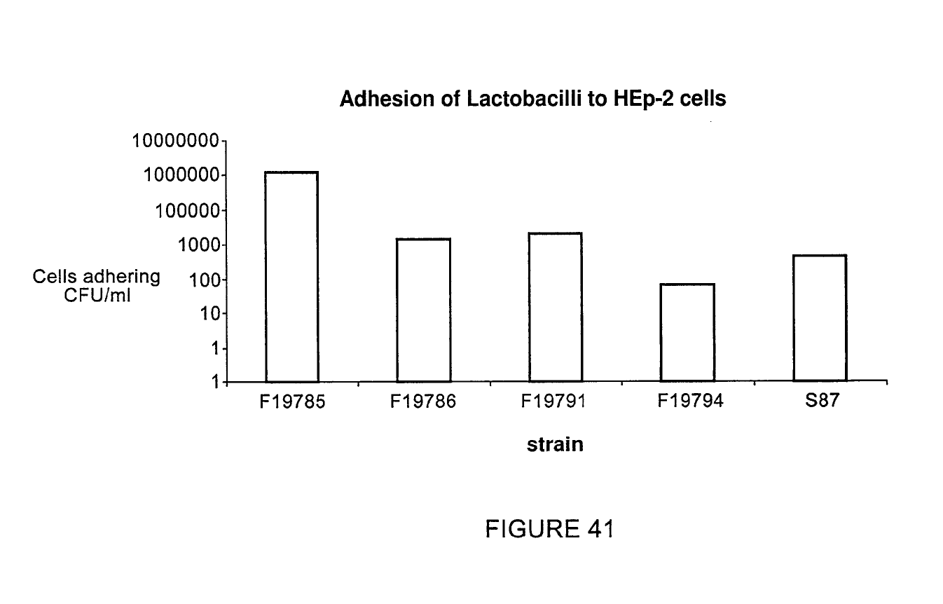

Results presented in FIG. 41 indicate that FI 9785 was the most adherent strain.

### Example 2b

### In vitro Competitive Exclusion of E. coli

Adhesion assays were conducted as described in Example 2 with the exception that the HEp-2 monlayers were inoculated with both the enteropathogen E. coli, and one of 8 Lactobacilli strains. Moreover, the adhesion of the enteropathogen was assessed, rather than the adhesion of the Lactobacilli. This was achieved by plating the serial dilutions onto LB agar plates. Three such assays were conducted, each in triplicate. The HEp-2 monolayers were inoculated with equal numbers (3×10⁸ cells) of E. coli and Lactobacilli CFU/ml were calculated and adhesion expressed as a percentage of the number of cells recovered from controls (normalised to 100%).

Results presented in FIG. 42: Strain FI9785 was a relatively good excluder of pathogenic E. coli in this system.

### Example 2c

### In Vitro Competitive Exclusion of E. Coli Using Gut Explants of SPF Chickens

This experiment was designed to compare the effectiveness of Lactobacilli strains in gut explants tissues. The experiments were conducted as described in Example 2b with the exception that tissue explants were used instead of HEp-2 cell line. Assays were performed as described previously Oyofo B A, Droleskey R E, Norman J O, Mollenhauer H H, Ziprin R L, Corrier D E, DeLoach J R. (1989) Inhibition by mannose of in vitro colonization of chicken small intestine by Salmonella typhimurium. Poultry Science 68, 1351-6. One day old SPF chicks were sacrificed and approximately 2 cm lengths of intestine were removed aseptically and added to 10 ml Sterile Kreb's Ringers solution. Gut tissues were then cut vertically to expose the epithelial surface. The sections were washed in 20 ml fresh sterile Kreb's Ringers and 1 ml of 1×10⁷ CFU each of the E. coli and Lactobacilli were added. The tubes were incubated at 37° C. with shaking at 225 rpm for 3 h. Sections were then rinsed 3 times in fresh Ringer's to remove non-adherent cells and then homogenised. Serial dilutions of the homogenates were plated on LB agar.

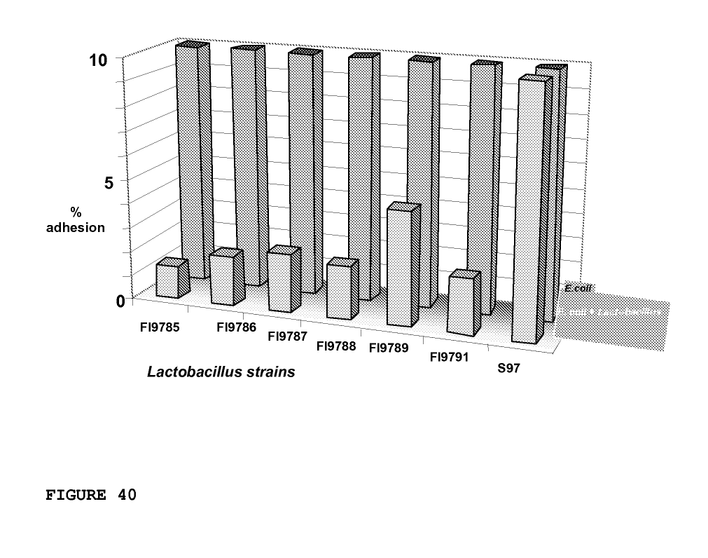

Results of the exclusion assay with this method are presented in FIG. 40 indicating that FI9785 was the most effective at excluding E. coli from attaching to the chick gut tissues.

### Example 3

L. johnsonii FI9785 PROTECTS FROM IN VIVO COLONISATION OF POULTRY BIRDS BY Campylobacter jejuni.

This experiment was designed to establish whether Lactobacillius johnsonii FI9785 exerts a protective effect in vivo in poultry birds.

60 one-day old SPF chicks were randomly divided into two groups of 30 (designated group 1 and group 2). All birds were housed in Wey-Isolators with food and water given ad libitum. At day old (day 1) group 1 was dosed with 1×10⁹ CFU of Lactobacillius johnsonii FI9785 (100 μl of a 16 hour broth (MRS) culture resuspended in PBS to give 1×10¹⁰ CFU/ml). Group 2 was left un-dosed.

At 2 days of age (day 2) birds in both groups were challenged with 1×10⁵ CFU Campylobacter jejuni resuspended in PBS given orally (100 μl of a 1×10⁶ CFU/ml).

At 1, 5, 7 and 14 days post challenge 5 birds from each group were randomly sacrificed by cervical dislocation and post-mortemed.

1 g tissue samples of liver, spleen, duodenum, jejunum, ileum, colon and caeca were aseptically removed and placed in 9 ml of PBS and homogenised. Ten fold serial dilutions (10¹-10⁶) were plated onto selective media (BASAC) and incubated microaerophilically. At the end of the experiment (35 days post challenge) all remaining birds (10/group) were sacrificed by cervical dislocation and full post-mortem examinations carried out and C. jejuni enumerated in the above mentioned tissues.

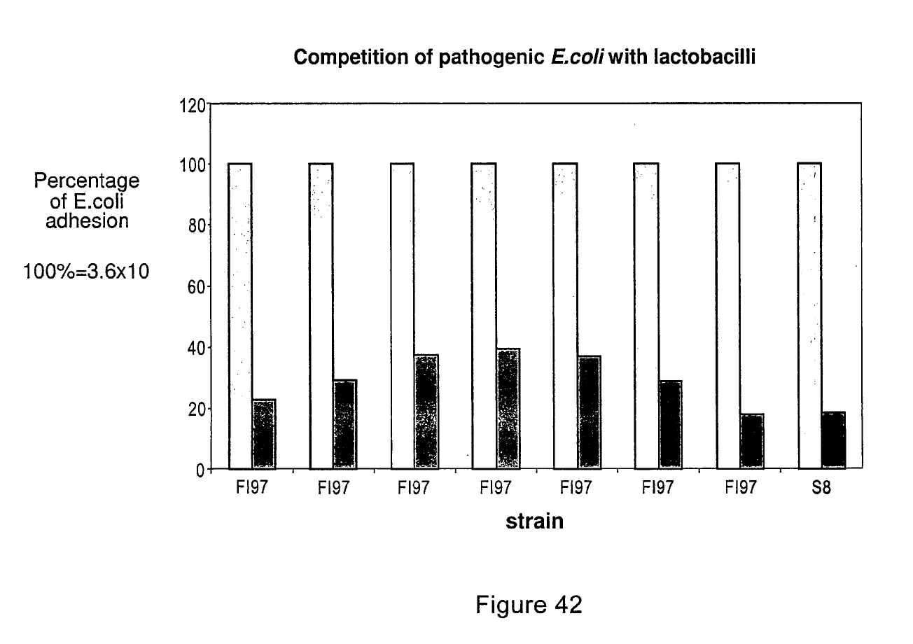

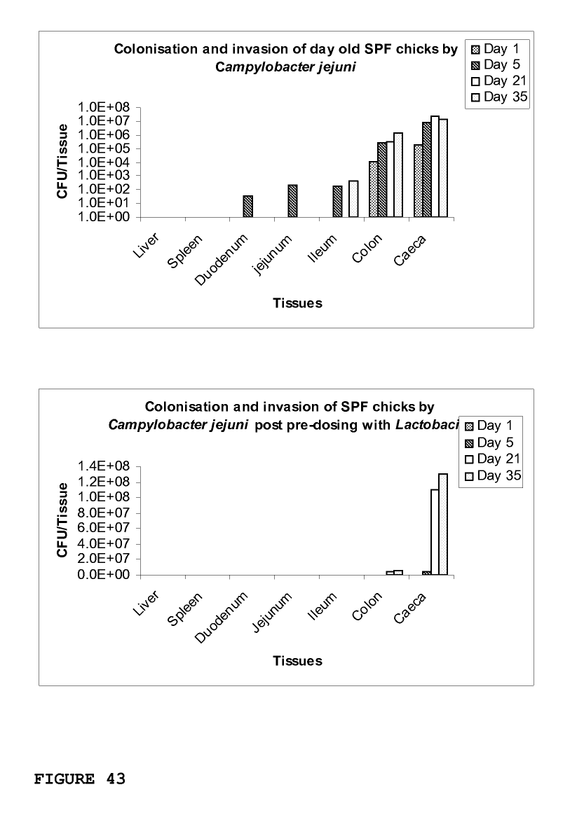

Results shown in FIG. 43 indicate that administration of L. johnsonii FI9785 prevents colonisation of C. jejuni in most of the tissues examined and delays colonisation of the caeca of poultry birds.

### Example 3b

### Exclusion of Camphlobacter by Lactobacillus johnsonii

### Method

Tagging of Lactobacillus johnsonii

In order to enumerate L. johnsonii in the gut of poultry by selection on MRS media the strain was tagged by marking it with resistance to the antibiotic chloramphenicol. L. johnsonii FI9785 is naturally resistant to the antibiotic neomycin sulphate. The addition of these two antibiotics in the enumeration medium was sufficient for selective growth of the L. johnsonii strain FI9785. Resistance to chloramphenicol was conveyed by transforming FI9785 with the plasmid pFI12431. Based upon the native plasmid p9785S, the construction of pFI2431 is described in Horn, N., Wegmann, U., Narbad, A., Gasson, M. J. (2005) Characterisation of a novel plasmid p9785S from Lactobacillus johnsonii FI9785. Plasmid 54 (2): p. 176-183a map is shown in FIG. 45

The resulting derivative of L. johnsonii FI9785 was grown on MRS media containing neomycin sulphate (10 □g/ml) and chloramphenicol (7.5 □g/ml).

Preparation of L. johnsonii

Cells of Lactobacillus grown overnight (16 h) were harvested and washed twice in PBS. The cells were then resuspended in PBS to give a final concentration of 1×10⁹ cells/ml for the subsequent administration to poultry birds.

### Campylobacter Challenge Experiments

Light Sussex (LSX) poultry birds were used. Light Sussex (LSX). Twenty-four birds were housed in 4 cages, 1 group per cage with six birds per group. The treatment groups were as follows:

2 cages per treatment:

Addition of
Campylobacter jejuni

| | | yes | yes |
|---|---|---|---|
| Lactobacillus | no | Group 1 | Group 2 |
| johnsonii | yes | Group 3 | Group 4 |

One day post hatching, each bird in Groups 3 and 4 were gavaged with 100 □l (1×10⁸ cells) of freshly prepared L. johnsonii. Birds in Groups 1 and 2 were given 100 □l of PBS only.

All chicks were housed on wood shavings and fed standard rations with tap water available ad libitum. On Day 8, the dosing regime was repeated.

On Day 13, one bird from each cage was selected at random, removed from the cage and killed for bacteriological analysis of the caecal content. The caecal contents were weighed, then resuspended in 10 volumes of PBS, homogenised and serial dilutions plated on MRS agar (containing 10 □g/ml neomycin sulphate and 7.5 □g/ml chloramphenicol) and CCDA agar for the enumeration of L. johnsonii and Campylobacter respectively.

On Day 13, all remaining birds in each group were challenged with Campylobacter jejuni strain 81-176 (−10⁸ CFU in 0.3 ml MH per bird).

On day 14 (1 day post challenge), one bird per group was removed for the enumeration of L. johnsonii and Campylobacter.

On day 20 (6 days post challenge), all remaining birds were removed for the enumeration of Campylobacter.

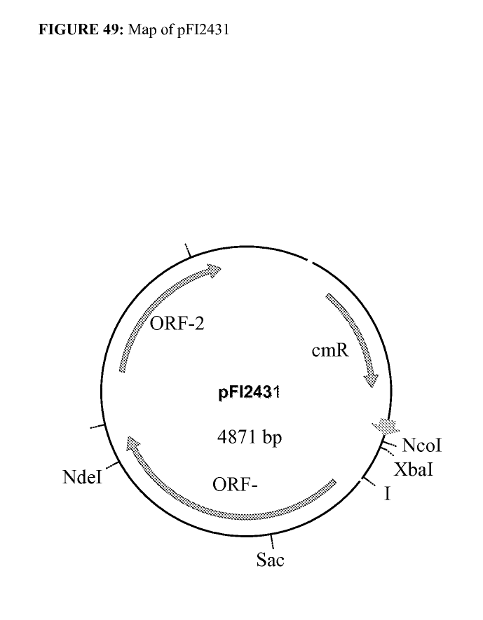

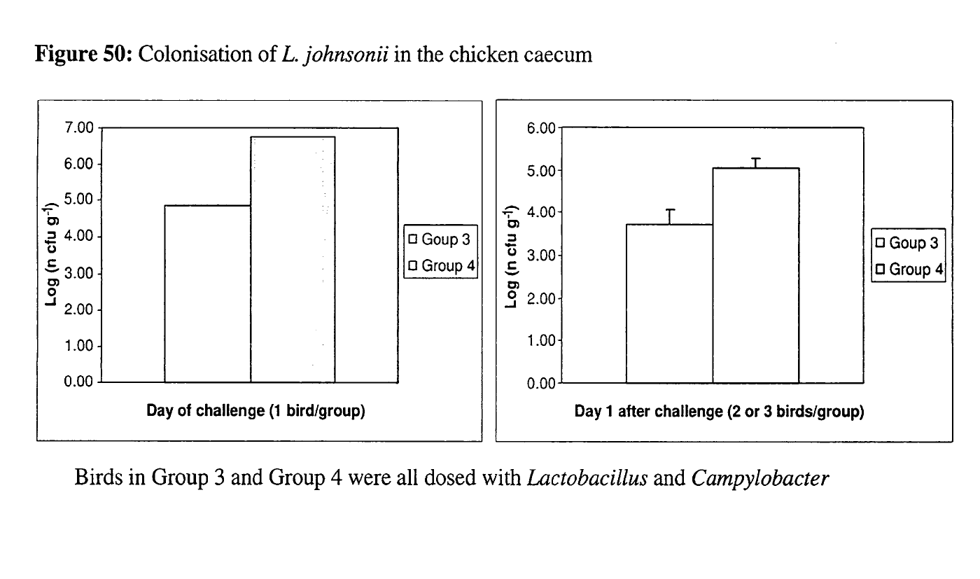

The results shown in FIGS. 49 and 50 indicate that in 50% of the birds that were treated with L. johnsonii a 4 log reduction in the levels of Campylobacter colonisation at day 6 after challenge was observed.

In the remaining 50% of the birds there was no reduction in Campylobacter levels. However it was observed that in this group the level of colonisation of Lactobacillus in the caecum was one to 2 orders of magnitude less than in the group where exclusion of Campylobacter was observed. It appears thatthere needs to be a minimal colonisation level (i.e >1×10⁵/g caecal content) for Lactobacillus to be an effective excluder of Campylobacter. As the birds were onlydosed on 2 days there is potential to increase the caecal colonisation levels of Lactobacillus by alteration of the dosage regimes if required.

Rosenquist, H., Nielsen, N. L., Sommer, H. M., Norrung, B., Christensen, B. B. (2003) Quantitative risk assessment of human campylobacteriosis associated with thermophilic Campylobacter species in chickens. Int J Food Microbiol. May 25; 83(1):87—has concludes that a 2 log reduction in numbers of Campylobacter on chicken carcasses could reduce human cases of Campylobacter infection by 30 times. Therefore L. johnsonii has the potential to be used as a chicken probiotic for reduction of Campylobacter levels with significant impact on the cases of Campylobacter infections in humans.

### Example 3c

### Exclusion of Eimeria by L. Johnsonii

### Introduction

Evidence to date indicates that Necrotic Entiritis (NE) (caused by Clostridium perfringens) in poultry is associated with intestinal stasis and lesions induced by coccidiosis. Coccidiosis is the most frequently diagnosed intestinal disease which is concurrent to NE infection, followed by clinical nemorrhagic enteritis and ascaridiasis. With the slow removal of anti-coccidial ionophores within the EU it is becoming even more important to find alternative methods to control the incidence and development of NEVan Immerseel, F., Rood, J. I., Moore, R. J., Titball, R. W., 2009, Rethinking our understanding of the pathogenesis of necrotic enteritis in chickens. Trends in Microbiology 17, 32-36. Williams, R. B., 2005, Intercurrent coccidiosis and necrotic enteritis of chickens: rational, integrated disease management by maintenance of gut integrity. Avian Pathology 34, 159-180. Lactobacillus johnsonii FI9785. Plasmid 54 176-183. Al-Sheikhly, F., Al-Saieg, A., 1980, Role of Coccidia in the occurrence of necrotic enteritis of chickens. Avian Dis 24, 324-333. Baba, E., Ikemoto, T., Fukata, T., Sasai, K., Arakawa, A., McDougald, L. R., 1997, Clostridialpopulation and the intestinal lesions in chickens infected with Clostridium perfringens and Eimeria necatrix. Veterinary Microbiology 54, 301-308. Yegani, M., Korver, D. R., 2008, Factors Affecting Intestinal Health in Poultry. Poult Sci 87, 2052-2063.

The coccidiosis in chickens is caused by the parasite Eimeria. Current sales of live coccidial vaccines, which are only used in broiler breeder flocks, are estimated at £15M pa. Coccidiosis is controlled in the UK's 850M broilers by anti-coccidial drugs. Alternative control measures are therefore essential. Here we assessed the potential of Lactobacillus in controlling the colonisation of the parasite.

Preparation of Lactobacillus johnsonii

Cells of L. johnsonii grown overnight (16 h) were harvested and washed twice in PBS. The cells were then resuspended in PBS to give a final concentration of 1×10⁹ cells/ml for the subsequent administration to poultry birds.

### Coccidiosis Challenge Experiments

The poultry birds used were Light Sussex chicks. Two groups of 10 one day old birds were housed in colony cages in separate rooms (rooms 1 and 2). One day post hatching all 10 birds in room 1 were inoculated with 100 ul (1×10⁸ cells) of freshly prepared L. johnsonii. All 10 birds in Room 2 were given 100 ul of PBS only. On Day 8, the Lactobacillus dosing regime was repeated. On day 14 all birds were moved to single bird cages labelled 1-10 and 11-20 but still kept in separate rooms. On day 20 all birds in both groups were inoculated with 1,000 sporulated Eimeria tenella oocysts of the Houghton strain. On days 23 to 26 faecal samples from each cage were collected and processed for presence of oocysts using the method essentially described by Long P, Joyner L, Millard B and Norton C. A guide to laboratory techniques used in the study and diagnosis of avian coccidiosis. Folia Veterinaria Latina 1976; 6: 201-217.

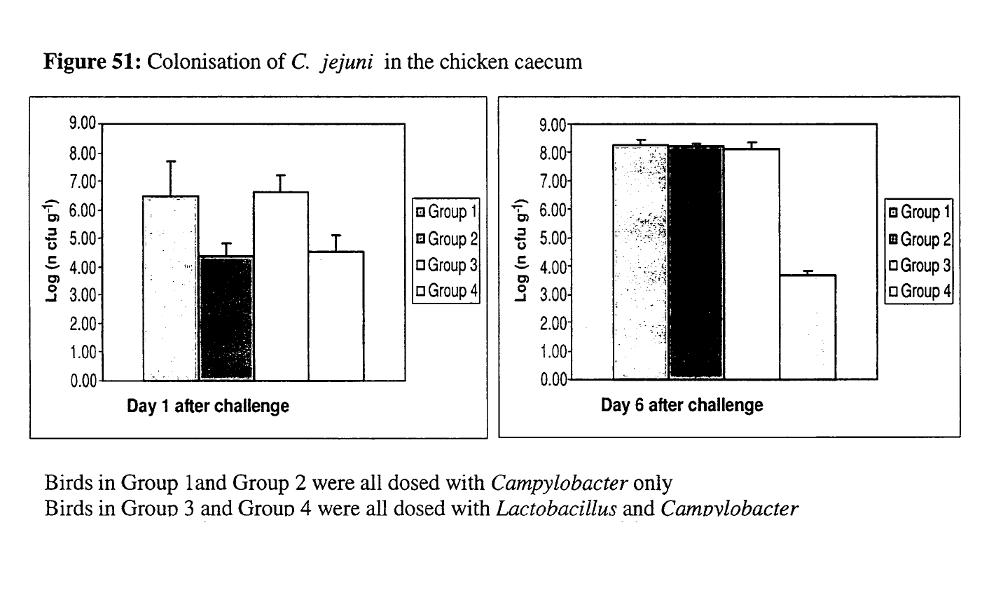

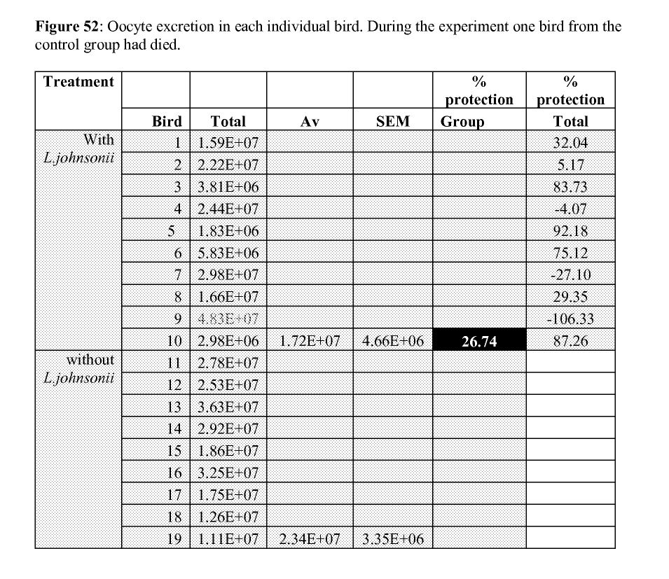

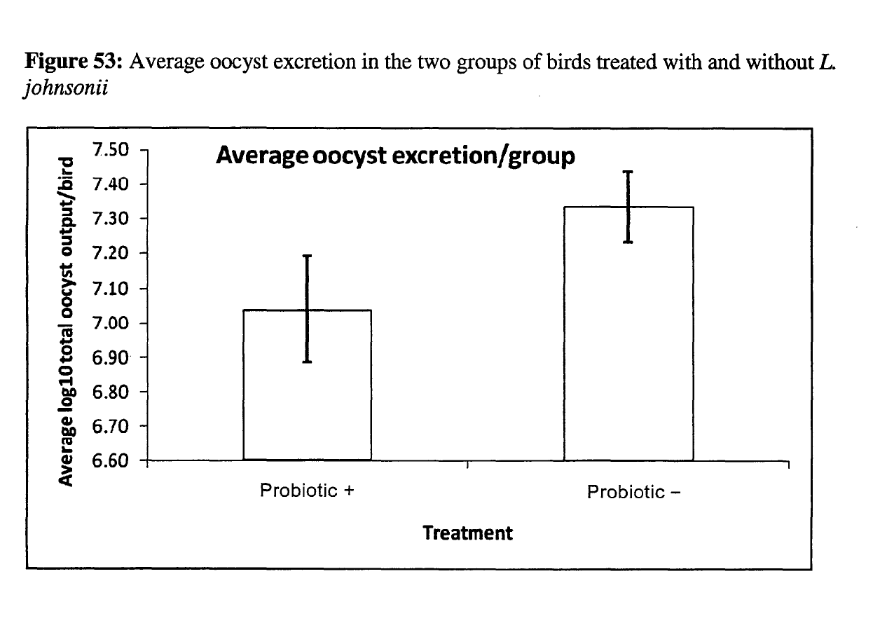

The results shown in FIGS. 51 and 49indicate that comparing both groups on average there was 26.74% reduction in oocyst excretion. Of the group that received the Lactobacillus asignificant reduction in 7/10 birds and an average reduction in these 7 birds of 57.8% was achieved. It is possible that in the remaining 3 birds Lactobacillus failed to colonise but as Lactobacillus colonisation levels were not measured it is difficult to say if this was the cause or whether these data reflect the variation in protection of different birds.

The effects of L. johnsonii on oocyst level observed here have benefits in four different categories.
1. A reduction in oocyst excretion will be economically relevant to the producer since subclinical coccidiosis can dramatically impact on FCR and associated parameters (body weight, etc).
2. Infections due to Eimeria has been associated with diseases such as necrotic enteritis—a reduction in Eimeria will help reduce the prevelance of NE.
3. Reduced oocyst excretion will also reduce environmental contamination, thus reducing the exposure of neighbour and subsequent birds.
4. Sterile protection is not necessarily essential—vaccination is based on controlled exposure to small numbers of wild-type or attenuated parasites. Reducing and not removing all oocysts will support immunisation by natural exposure.

### Example 4

### Cloning, Sequencing and Biological Activity of L. Johnsonii FI9785 Eps Cluster Sequences

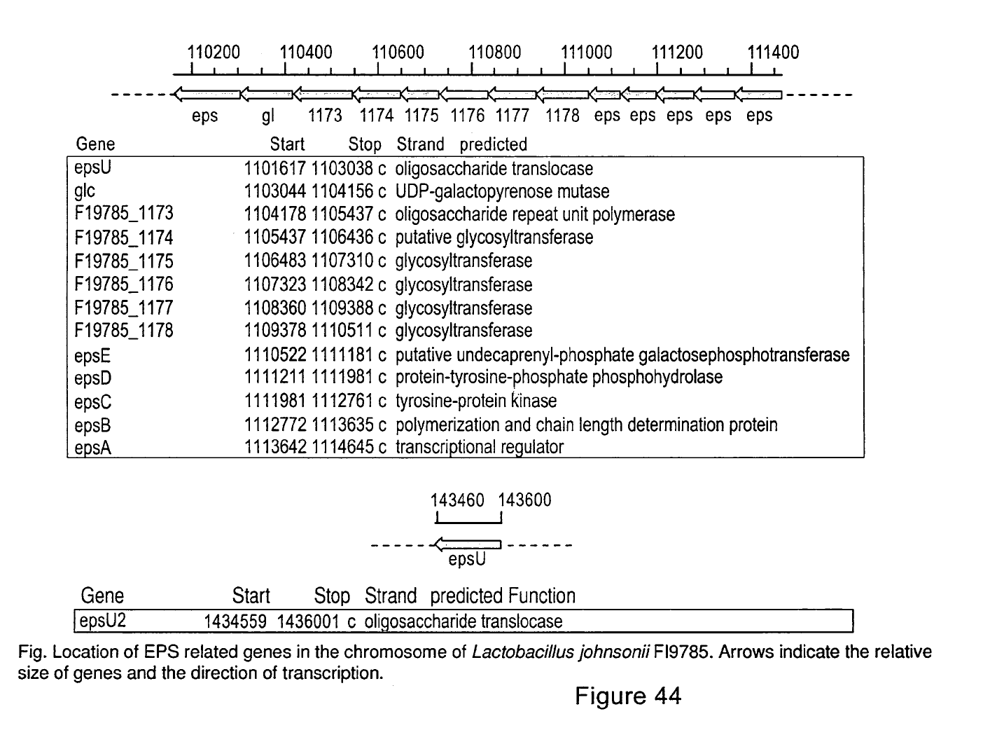

In the L. johnsonii FI9785 strain, the gene clusters coding for production of secreted EPSs are located on a 11.6-kbp chromosomal DNA, containing 14 genes, designated epsA, B, C, D, E, U and other novel genes which are annotated as unk, the unidentified putative esp genes coding for transferases (unpublished data). See the Map, FIG. 44; the Map is not to scale.

The entire EPS gene cluster from L. johnsonii FI9785 has been cloned, sequenced and the putative amino acid sequence obtained as follows:

| SEQ ID. | DESCRIPTION - | | SEQ ID. |
|---|---|---|---|
| NO. NUC. | FI9785 | | NO AMINO |
| ACID SEQ. | GENE NUMBER | FUNCTION | ACID SEQ. |
| 11 | 1183 | EPSA | 25 |
| 12 | 1182 | EPSB | 26 |
| 13 | 1181 | EPSC | 27 |
| 14 | 1180 | EPSD | 28 |
| 15 | 1179 | EPSE | 29 |
| 16 | 1178 | unknown | 30 |
| 17 | 1177 | GLYCOSYL- | 31 |
| | | TRANSFERASE | |
| 18 | 1176 | GLYCOSYL- | 32 |
| | | TRANSFERASE | |
| 19 | 1175 | GLYCOSYL- | 33 |
| | | TRANSFERASE | |
| 20 | 1174 | GLYCOSYL- | 34 |
| | | TRANSFERASE | |
| 21 | 1173 | OLIGO-REPEAT | 35 |
| | | UNIT | |
| | | POLYMERASE | |
| 22 | 1172 | ? | 36 |
| 23 | 1171 | EPSU | 37 |
| 24 | 1170 | | 38 |

When the epsC gene is mutated there is a loss in adhesion functionality of L. Johnsonii FI9785.

Conversely, when this gene is introduced into a strain which does not exhibit either good adhesion ability or the ability to competitively exclude pathogenic microorganisms, these sequences have been found to confer both good adhesion ability and the ability to competitively exclude pathogenic microorganisms.

The mucin binding proteins of L. johnsonii FI9785 have been cloned, sequenced and the putative amino acid sequence obtained as follows:

| SEQ ID. | DESCRIPTION - | | SEQ ID. |
|---|---|---|---|
| NO. NUC. | FI9785 | | NO AMINO |
| ACID SEQ. | GENE NUMBER | FUNCTION | ACID SEQ. |
| 1 | 111 | HYPOTHETICAL | 6 |
| | | PROTEIN | |
| 2 | 1070 | MUCUS BINDING | 7 |
| | | PROTEIN | |
| 3 | 1481 | PSEUDOGENE | 8 |
| 4 | 1482 | MUCUS BINDING | 9 |
| | | PROTEIN | |
| 5 | 1651 | PSEUDOGENE | 10 |

### Example 5

### Over-Expression of epsC in the Lactobacillus johnsonii Smooth Colony Variant Strain FI10386

An expression vector pFI2560 based upon the Lactobacillus johnsonii plasmid p9785sp (Horn N., Wegmann U., Narbad A. & Gasson M. J. (2005). Characterisation of a novel plasmid p9785S from Lactobacillus johnsonii FI9785. Plasmid 54 176-183) was constructed as follows. An 890 bp fragment conveying resistance to chloramphenicol was PCR amplified using primers 5′-TGCGCACCCATTAGTTCAACAAACG-3′ and 5-CCAACTAACGGGGCAGGTTAGTGAC-3′ from pUK200 (Wegmann, U. J. R. Klein, I. Drumm, O. P. Kuipers and B. Henrich (1999), Introduction of peptidase genes from Lactobacillus delbrueckii subsp. lactis into Lactococcus lactis and controlled expression, Appl. Environ. Microbiol. 65 4729-4733) and cloned into the MscI site of p9785S (p9785 cm). A 50 bp translational-fusion linker was formed by annealing primers 5′-GATATCAGAAAGGAGGTTCAGTCCATGGAGTACTTAGATAGCTAAGCGCT-3′ and 5′-AGCGCTTAGCTATCTAAGTACTCCATGGACTGAACCTCCTTTCTGATATC-3′ and cloned into the MscI site of p9785 cm (p9785 cmTF). A 183 bp XbaI-XhoI terminator region from pUK200 was cloned as a blunt-ended fragment into the EcoR471II site of p9785 cmTF (p9785 cmTFter). Finally primer pair 5′-AGTTCTTAGCTCCTATTTTTTTGCCC-3′ and 5′-TTGATAAATTCGATTTGAATTATTTGTTTCGTC-3′ was used to amplify the PapfI promoter region associated with the aggregation promoter factor (Ventura M, Jankovic I, Walker D C, Pridmore R D, Zink R (2002). Identification and characterization of novel surface proteins in Lactobacillus johnsonii and Lactobacillus gasseri. Appl Environ Microbiol 68:6172-81) from the Lb. gasseri strain NCIMB 11718 (ATCC 33323). The 208 bp promoter fragment was cloned into the EcoRV site of p9785 cmTFter to create the expression vector pFI2560.

Primer pair 5′-ATCCATGGGATTGTTTAATAGACG-3′ and 5′-TTATTTATTACTTCGTTTCTGTATC-3′ was used to PCR amplify a fragment coding for the EpsC protein using genomic DNA of either Lactobacillus johnsonii FI9785 or the smooth colony variant FI10386 as template. Each 784 bp fragment was digested with NcoI and cloned into NcoI and ScaI digested pFI2560 to create pFI2660 and pFI2659 respectively. Lb. johnsonii FI10386 transformed with either pFI2560 (vector control), pFI2660 (EpsC rough) or pFI2659 (EpsC smooth) generated FI10774, FI10773 and FI10772 respectively. The EpsC over-expressing isolates and the control strain were streaked onto MRS agar containing chloramphenicol (7.5 μg/ml) and the morphology of colonies was observed (FIG. 45). The colony morphology obtained for both the vector control strain FI10774 and the strain over-expressing EpsC smooth FI10772 was consistent with that of the original smooth colony variant parental strain FI10386 (FIG. 46). In the case of the EpsC rough over-expressing strain FI10773, an irregular or rough-edge colony morphology consistent with the wild type strain FI9785 (FIG. 46) was clearly apparent.

### Example 6

The Role of exopolysaccharide (EPS) in Adhesion phenotype of FI9785

In order to establish if the esp genes were important in the adhesion phenotype of FI9785, the ability of this strain and its derivatives were tested for their ability to adhere to human HT29 cell line.

Methods: A modified radioactive labelling method based on that described by Vesterlund S., Paltta M. K., Karp, M., and Ouwehand, A. C. 2005. measurement of bacterial adhesion-in vitro evaluation of different methods. J. Microbiol. Methods. 60 225-233 was used. Bacterial cells were grown overnight bacterial cells in 20 ml of MRS broth at 37° C. Inoculated 0.5 ml of the overnight culture and 12.5 μl of ³H Thymidine (0.46 MBq) into 3.5 ml MRS. Incubated at 37° C. for 1-3 hours to allow optimum uptake and incorporation of ³H Thymidine into the bacterial DNA. Centrifuged at 12,000 rpm for 4 minutes and discarded the supernatant. Washed the pellets 3 times in PBS to remove the unincorporated ³H Thymidine. Added 1 ml of Tissue culture media without antibiotic. (TCMWA) 20 ul of the cell suspension was used to measure viable counts. Adhesion assays were performed with 24 well plates with confluent HT29 monolayers. The monolayers were washed 3 times with TCMWA and to each well 1 ml of the ³H Thymidine labelled bacteria (1×10⁸ cells/ml) was added and plates incubated in TCMWA suspension and seal the lids with tape incubated at 37° C. in an atmosphere of 5% CO₂ for 3 hours. The unbound bacterial cells were removed by washing three times in TCMWA. 1 ml Trypsin solution was added to each well to dislodge the monolayer and the cell suspension was added directly to scintillation vial. The radioactivity in each vial was measure after addition of Scintisafe.

Results shown below indicate that compared to the wild type FI9785 the smooth variant (FI10386) with mutation in the epsC gene had significantly reduced capacity to adhere to the mammalian tissue cell line. Expression of the wild type epsC gene in the smooth variant (FI10773) resulted in partial restoration of the adhesion capacity and this restoration was not observed when the smooth variant was transformed with only the vector (FI10774).

| | FI5876 | FI10386 | FI10773 | FI10774 |
|---|---|---|---|---|
| Adhesion | 5.3 × 10⁵ | 4.8 × 10⁴ | 1 × 10⁵ | 2 × 10⁴ |
| number of | | | | |
| Cells/well | | | | |
| % adhesion | 100 | 9 | 20 | 4 |
| compared to | | | | |
| the wild type | | | | |

Relative adhesion of FI9785 and its derivatives to HT29 cell culture.

### Strains

Lb. johnsonii FI9785 Wild-type

Lb. johnsonii FI10386 FI9785, smooth colony variant
Lb. johnsonii FI10773 FI10386 carrying pFI2660 (pFI2560 P_{apfi}::eSPC^{FI9785})
Lb. johnsonii FI10774 FI10386 carrying pFI2560 (Cm^{r}, P_{apfi}, p9785S replicon)

1-26. (canceled)

27. A culture of a Lactobacillus species or strain comprising:
(i) a portion of the EPS gene cluster base sequence set forth in anyone of SEQ ID NOS 11, 12, 13, 14, 15, 16, 17, 18, 19, 20, 21, 22, 23 and 24, or
(ii) a mucin binding protein having the nucleotide base sequence set forth in anyone of SEQ ID NOS 1, 2, 3, 4 and 5.

28. The culture according to claim 27, wherein the Lactobacillus species is Lactobacillus johnsonii or Lactobacillus gasseri.

29. The culture according to claim 27, wherein the Lactobacillus strain is L. johnsonii FI9785.

30. The culture according to claim 27, wherein the Lactobacillus strain is deposited with NCIMB as deposit number NCIMB 41632.

31. The culture according to claim 27, wherein the culture is a monoculture.

32. The culture according to claim 27, wherein the culture is a mixed culture.

33. A food composition comprising a culture according to claim 27.

34. A method of restricting the colonization of a vertebrate gut by one or more pathogens comprising administering a protectively effective amount of a composition comprising a live culture L. johnsonii according claim 27.

35. The method of claim 34, wherein the composition further comprises live B. subtilis.

36. The method of claim 34, wherein the pathogen is C. perfringens, E. coli, or a Campylobacter.

37. A method of prophylaxis against necrotis entiritis comprising administering a protectively effective amount of a composition comprising a live culture L. johnsonii according claim 27.

38. A method of improving one or more of weight gain, feed conversion, and immune competency of immature vertebrates comprising administering a composition comprising a live culture L. johnsonii according claim 27.

39. The method of claim 33, wherein the vertebrate is selected from the group consisting of: humans, bovine, ovine, porcine, equine, avian, pets and companion animals.

40. An isolated nucleic acid comprising:
(i) a portion of the EPS gene cluster base sequence as set forth in any one of SEQ ID NOS 11, 12, 13, 14, 15, 16, 17, 18, 19, 20, 21, 22, 23 and 24;
(ii) a nucleotide sequence encoding a protein or peptide sequence as set forth in any one of SEQ ID NOS 25-38;
(iii) a mucin binding protein gene having a nucleotide base sequence as set forth in any one of SEQ ID NOS 1, 2, 3, 4 and 5; or
(iv) a nucleotide sequence encoding a protein or peptide sequence as set forth in any one of SEQ ID NOS 6-10.

41. The isolated nucleic acid of claim 40, comprising a nucleic acid sequence selected from the group consisting of SEQ ID NOS 1-5 and SEQ ID NOS 11-24.

42. A method for conferring enhanced adhesion to a bacterium comprising introducing into said bacterium an isolated nucleic acid according to claim 40.

43. A method of identifying a candidate organism for competitively excluding pathogens when introduced into the GI tract of a vertebrate comprising hybridizing nucleic acids from said candidate organism to a nucleic acid sequence of claim 41.

44. A protein or peptide sequence comprising the amino acid sequence as set forth in anyone of SEQ ID NOS 6-10 or SEQ ID NOS 25-38.

45. A method of identifying a candidate organism for competitively excluding pathogens when introduced into the GI tract of a vertebrate comprising raising antibodies to an immunogenic portion of a protein or peptide sequence of claim 44 and using the antibodies to identify candidate organisms which react with said antibodies.

46. A coccidiostat comprising the culture of Lactobacillus species or strain of claim 27.

47. A probiotic comprising the culture of Lactobacillus species or strain of claim 27.
